(19) United States
(12) Patent Application Publication
(10) Pub. No.: US 2020/0400673 A1
(43) Pub. Date: Dec. 24, 2020

(54) METHODS FOR CANCER DETECTION BY EVALUATION OF GLYCAN-BINDING PATTERNS OF IMMUNOGLOBULINS IN GASTROINTESTINAL LAVAGE FLUID SAMPLES

(71) Applicant: Creatics LLC, Rockland, MA (US)

(72) Inventors: Lewis K. Pannell, Mobile, AL (US); Joseph John Otto, Norfolk, VA (US)

(73) Assignee: Creatics LLC, Rockland, MA (US)

(21) Appl. No.: 17/010,069

(22) Filed: Sep. 2, 2020

Related U.S. Application Data
(63) Continuation of application No. PCT/US2019/025616, filed on Apr. 3, 2019.
(60) Provisional application No. 62/761,711, filed on Apr. 3, 2018.

(51) Int. Cl. G01N 33/574; A61K 31/702; A61K 9/00; G01N 33/533

(52) U.S. Cl. CPC G01N 33/57438; A61K 31/702; G01N 2800/7028; G01N 33/533; A61K 9/0095

(57) ABSTRACT

Described herein are non-invasive or minimally invasive methods and compositions for collecting and assessing samples for detection of gastrointestinal (GI) tract cancers and pancreatic cancers. Samples comprising gastrointestinal lavage fluid (GLF) are obtained from subjects and screened against a glycan microarray to reveal disease-specific glycan-binding patterns by immunoglobulins contained in the GLF, especially immunoglobulin A (IgA). The disease-specific glycan-binding patterns include a disease-specific glycan or subset of glycans from the microarray that are bound by the immunoglobulins in GLF at highest intensities or at lowest intensities, as well as one or more glycan motifs found within this disease-specific subset of glycans.

### CROSS REFERENCE TO A RELATED APPLICATION

This application claims priority to U.S. Provisional Application No. 62/761,711, filed Apr. 3, 2018, the disclosure of which is incorporated herein by reference in its entirety.

### FIELD OF THE INVENTION

The present disclosure relates to non-invasive or minimally invasive methods for the detection and prognosis of cancers such as pancreatic cancers and colon/colorectal cancers.

### BACKGROUND

Disorders associated with the gastrointestinal (GI) and hepatobiliary tracts and the organs/tissues associated with the GI tract include cancers such as gastric cancer, esophageal cancer, liver cancer, and pancreatic cancer. Pancreatic cancer (e.g., pancreatic adenocarcinoma), in particular, is a malignant growth of the pancreas that mainly occurs in the cells of the pancreatic ducts. This disease is the ninth most common form of cancer, yet it is the fourth and fifth leading cause of cancer deaths in men and women, respectively. Cancer of the pancreas is almost always fatal, with a five-year survival rate that is less than 3%.

The most common symptoms of pancreatic cancer include jaundice, abdominal pain, and weight loss, which, together with other presenting factors, are often nonspecific in nature. Thus, diagnosing pancreatic cancer at an early stage of tumor growth is often difficult and requires extensive diagnostic work-up, often times incidentally discovered during exploratory surgery. Endoscopic ultrasonography is an example of a non-surgical technique available for diagnosis of pancreatic cancer. However, reliable detection of small tumors, as well as differentiation of pancreatic cancer from focal pancreatitis, is difficult. The vast majority of patients with pancreatic cancer are presently diagnosed at a late stage when the tumor has already extended beyond the pancreas to invade surrounding organs and/or has metastasized extensively. Late detection of the disease is common with the majority of patients being diagnosed with advanced disease that often results in death; only a minority of patients are detected with early stage disease.

Ideally, biomarker detection would utilize samples, such as biopsy tissue or fluids, that are specifically associated with the organ of interest. Pancreatic juice fits this as it is secreted directly pancreas. However, the fluid can only be obtained by invasive means. Invasive techniques to diagnose pancreatic cancers and disorders and diseases related to the GI tract are inconvenient and expose a subject to significant risk. Examples of non-invasive methods to identify patients with disorders of the gastrointestinal tract or associated organs/tissues are described in International Patent Application Nos. PCT/US2011/051269 and PCT/US2014/056847, the disclosures of which are incorporated herein by reference herein in their entireties. Nonetheless, there remains a need for additional methods for the detection and prognosis of cancers that are difficult to detect at early stages, such as pancreatic cancers and colon/colorectal cancers.

Within the context of the glycan-based cancer biomarkers, carbohydrate antigen 19-9 (CA 19-9) which is found in the serum, is in the forefront for pancreatic cancer. However, the use of CA19-9 as a screening test for pancreatic cancer is strongly discouraged. For example, assessing CA19-19 is prone to being falsely normal (false negative) in many cases including pancreatic cancer patients with large tumors, or abnormally elevated in people who have no cancer at all (false positive). The main use of CA19-9 is therefore a surrogate biomarker for pancreatic cancer management. Further, the elevation of CA19-9 is not specific to pancreatic cancer, as the antigen can be elevated in many GI tract cancers (e.g., colorectal cancer, esophageal cancer, hepatocellular carcinoma) and even other non-cancer GI tract diseases (e.g., pancreatitis, cirrhosis, bile duct obstruction).

### SUMMARY OF INVENTION

In one aspect, the present disclosure relates to a method for diagnosing pancreatic cancer or predisposition thereto in a subject, by: obtaining a sample including gastrointestinal lavage fluid (GLF) from the subject, wherein the GLF includes immunoglobulin A (IgA); contacting the GLF with a plurality of glycans; and detecting binding between the IgA and the plurality of glycans, thereby detecting a glycan-binding pattern; wherein the glycan-binding pattern is associated with pancreatic cancer or predisposition thereto.

In certain embodiments, the glycan-binding pattern includes at least one glycan associated with pancreatic cancer or predisposition thereto. In some embodiments, the glycan-binding pattern includes at least two glycans associated with pancreatic cancer or predisposition thereto.

In certain embodiments, the glycan-binding pattern includes a glycan motif found in at least one glycan associated with pancreatic cancer or a predisposition thereto. In one embodiment, the glycan motif is GlcNAcα1-4Galβ1-4GlcNAc.

In some embodiments, the at least one glycan is pre-determined to be bound at the highest intensity by the IgA in GLF from pancreatic cancer patients. In some embodiments, the at least two glycans are pre-determined to be bound at the highest and the second highest intensities by the IgA in GLF from pancreatic cancer patients. In one embodiment, the at least one glycan or the at least two glycans have an intensity of higher than 10 RFU. In one embodiment, the at least one glycan or the at least two glycans are at at least the 90^{th} or 95^{th} percentile of intensities measured of the binding between the IgA and the plurality of glycans. In one embodiment, the at least one glycan or the at least two glycans are selected from the group consisting of:

### GlcNAcα1-4Galβ1-4GlcNAcβ1-3Galβ1-4GlcNAcβ1-3Galβ1-4GlcNAc; GlcNAcα1-4Galβ1-4GlcNAc;

### GlcNAcα1-4Galβ1-4GlcNAcβ1-3Galβ1-4Glc;

### GlcNAcα1-4Galβ1-4GlcNAcβ1-3Galβ1-4(Fucα1-3)GlcNAcβ1-3Galβ1-4(Fucα1-3)GlcNAc;

### GlcNAcα1-4Galβ1-4GlcNAcβ1-3Galβ1-4GlcNAc; and

### GlcNAcβ1-3Galβ1-4GlcNAcβ1-3Galβ1-4GlcNAcβ1-2Manα1-6(GlcNAcβ1-3Galβ1-4GlcNAcβ1-3Galβ1-4GlcNAcβ1-2Manα1-3)Manβ1-4GlcNAcβ1-4GlcNAc.

In some embodiments, the at least one glycan is pre-determined to be bound at the lowest intensity by the IgA in GLF from pancreatic cancer patients. In some embodiments, the at least two glycans are pre-determined to be bound at the lowest and the second lowest intensities by the IgA in GLF from pancreatic cancer patients. In one embodiment, the at least one glycan or the at least two glycans have an intensity of no higher than 7 RFU. In one embodiment, the at least one glycan or the at least two glycans are at or below the 10^{th} or 5^{th} percentile of all intensities measured of the binding between the IgA and the plurality of glycans. In one embodiment, the at least one glycan or the at least two glycans are selected from the group consisting of:

### GlcNAcβ1-6Galβ1-4GlcNAc;

### GlcA;

### Galβ1-4(Fucα1-3)(6S)Glc; and

### Manα1-6(Manα1-3)Manα1-6(Manα1-3)Man.

In some embodiments, the glycan-binding pattern comprises at least one glycan associated with pancreatic cancer or a predisposition thereto at a statistical significance testing p-value of ≤1.00e-6. In some embodiments, the glycan-binding pattern comprises at least one glycan having an area under curve (AUC) value of a receiver operating characteristic (ROC) curve of at least 0.95.

In certain embodiments, the plurality of glycans are exogenous glycans. In one embodiment, the plurality of glycans does not include an endogenous carbohydrate cancer antigen. In one embodiment, the plurality of glycans does not include the carbohydrate cancer antigen CA19-9.

In some embodiments, the pancreatic cancer is selected from the group consisting of pancreatic ductal adenocarcinoma (PDAC), adenosquamous carcinoma, squamous cell carcinoma, giant cell carcinoma, acinar cell carcinoma, small cell carcinoma, islet cell tumors, pancreas endocrine tumors (PETs), pancreatic neuroendocrine tumors (PNETs), insulinomas, glucagonomas, somatostatinomas, gastrinomas, VlPomas (arising from vasoactive intestinal peptide-making cells) and non-secreting islet tumors of the pancreas. In one embodiment, the pancreatic cancer is pancreatic ductal adenocarcinoma (PDAC).

In some embodiments, the method of the invention includes administering a lavage composition to the subject to thereby induce purgation of the subject's gastrointestinal tract. In some embodiments, the method of the invention includes administering a lavage composition to the subject to thereby cleanse the subject's gastrointestinal tract. In one embodiment, the lavage composition is administered orally. In one embodiment, the lavage composition is a solution or a solid composition that is reconstituted in a solvent prior to being administered to the subject.

In certain embodiments, the lavage composition includes an active ingredient selected from group consisting of polyethylene glycol (PEG), magnesium sulfate, sodium sulfate, potassium sulfate, magnesium citrate, sodium citrate, potassium citrate, ascorbic acid, citric acid, sodium picosulfate, sodium chloride, potassium chloride, bisacodyl, magnesium oxide, magnesium phosphate, potassium phosphate, sodium phosphate, monobasic sodium phosphate, dibasic sodium phosphate, potassium hydrogen tartrate, magnesium carbonate, magnesium hydroxide, sodium hydroxide, potassium hydroxide, sodium carbonate, sodium bicarbonate, ammonium carbonate, an anhydrous form thereof, a hydrate thereof, or a combination thereof.

In some embodiments, the lavage composition includes:

a PEG having a molecular weight of between about 3000 and about 4000, sodium sulfate, sodium bicarbonate, sodium chloride, and potassium chloride; or
a PEG having a molecular weight of between about 3000 and about 4000, sodium bicarbonate, sodium chloride, and potassium chloride; or
sodium sulfate, magnesium sulfate, and potassium sulfate; or
monobasic sodium phosphate and dibasic sodium phosphate, or a hydrate thereof.

In some embodiments, the method of the invention further includes separating the GLF from the sample. In some embodiments, the GLF does not comprise solid or semisolid fecal matter, fecal proteins, and fecal immunoglobulins. In some embodiments, the method involves removing solid or semisolid fecal matter, fecal proteins and fecal immunoglobulins.

In some embodiments, the binding between the IgA and the plurality of glycans is detected and measured using an immunoassay, a chemiluminescent assay, a fluorescent assay, a colorimetric assay, or a mass spectrometry assay.

In another aspect, the present disclosure relates to a method for detecting a glycan-binding pattern associated with pancreatic cancer or predisposition thereto in a subject, by: obtaining a sample including gastrointestinal lavage fluid (GLF) from the subject, wherein the GLF includes immunoglobulin A (IgA); contacting the GLF with a plurality of glycans; and detecting binding between the IgA and the plurality of glycans, thereby detecting the glycan-binding pattern.

All embodiments of the method for detecting a glycan-binding pattern described above are as set forth above for the method for diagnosing pancreatic cancer or predisposition thereto in a subject.

In another aspect, the present disclosure relates to a method for evaluating or monitoring the progression of pancreatic cancer or the efficacy of a pancreatic cancer treatment in a subject via a glycan-binding pattern associated with pancreatic cancer or predisposition thereto, by: obtaining a sample including gastrointestinal lavage fluid (GLF) from the subject, wherein the GLF includes immunoglobulin A (IgA); contacting the GLF with a plurality of glycans; and detecting binding between the IgA and the plurality of glycans, thereby detecting the glycan-binding pattern.

All embodiments of the method for evaluating or monitoring the progression of pancreatic cancer or the efficacy of a pancreatic cancer treatment described above are as set forth above for the method for diagnosing pancreatic cancer or predisposition thereto in a subject.

In a further aspect, the present disclosure relates to a method for diagnosing a gastrointestinal (GI) tract cancer or predisposition thereto in a subject, by: obtaining a sample including gastrointestinal lavage fluid (GLF) from the subject, wherein the GLF includes immunoglobulin A (IgA); contacting the GLF with a plurality of glycans; and detecting binding between the IgA and the plurality of glycans, thereby detecting a glycan-binding pattern; wherein the glycan-binding pattern is associated with the GI tract cancer or predisposition thereto.

In some embodiments, the GI tract cancer is pancreatic cancer. In one embodiment, the pancreatic cancer is selected from the group consisting of pancreatic ductal adenocarcinoma (PDAC), adenosquamous carcinoma, squamous cell carcinoma, giant cell carcinoma, acinar cell carcinoma, small cell carcinoma, islet cell tumors, pancreas endocrine tumors (PETs), pancreatic neuroendocrine tumors (PNETs), insulinomas, glucagonomas, somatostatinomas, gastrinomas, VlPomas (arising from vasoactive intestinal peptide-making cells) and non-secreting islet tumors of the pancreas. In one embodiment, the pancreatic cancer is pancreatic ductal adenocarcinoma (PDAC).

In some embodiments, the GI tract cancer is colon cancer. In some embodiments, the GI tract cancer is colon adenoma.

In certain embodiments, the glycan-binding pattern includes at least one glycan associated with the GI tract cancer or predisposition thereto. In some embodiments, the glycan-binding pattern includes at least two glycans associated with the GI tract cancer or predisposition thereto.

In certain embodiments, the glycan-binding pattern includes a glycan motif found in at least one glycan associated with pancreatic cancer or a predisposition thereto. In one embodiment, the glycan motif is GlcNAcα1-4Galβ1-4GlcNAc.

In some embodiments, the at least one glycan is pre-determined to be bound at the highest intensity by the IgA in GLF from pancreatic cancer patients. In some embodiments, the at least two glycans are pre-determined to be bound at the highest and the second highest intensities by the IgA in GLF from pancreatic cancer patients. In one embodiment, the at least one glycan or the at least two glycans have an intensity of higher than 10 RFU. In one embodiment, the at least one glycan or the at least two glycans are at at least the 90^{th} or 95^{th} percentile of intensities measured of the binding between the IgA and the plurality of glycans. In one embodiment, the at least one glycan or the at least two glycans are selected from the group consisting of:

### GlcNAcα1-4Galβ1-4GlcNAcβ1-3Galβ1-4GlcNAcβ1-3Galβ1-4GlcNAc;

### GlcNAcα1-4Galβ1-4GlcNAc;

### GlcNAcα1-4Galβ1-4GlcNAcβ1-3Galβ1-4Glc;

### GlcNAcα1-4Galβ1-4GlcNAcβ1-3Galβ1-4(Fucα1-3)GlcNAcβ1-3Galβ1-4(Fucα1-3)GlcNAc;

### GlcNAcα1-4Galβ1-4GlcNAcβ1-3Galβ1-4GlcNAc; and

### GlcNAcβ1-3Galβ1-4GlcNAcβ1-3Galβ1-4GlcNAcβ1-2Manα1-6(GlcNAcβ1-3Galβ1-4GlcNAcβ1-3Galβ1-4GlcNAcβ1-2Manα1-3)Manβ1-4GlcNAcβ1-4GlcNAc.

In some embodiments, the at least one glycan is pre-determined to be bound at the lowest intensity by the IgA in GLF from pancreatic cancer patients. In some embodiments, the at least two glycans are pre-determined to be bound at the lowest and the second lowest intensities by the IgA in GLF from pancreatic cancer patients. In one embodiment, the at least one glycan or the at least two glycans have an intensity of no higher than 7 RFU. In one embodiment, the at least one glycan or the at least two glycans are at or below the 10^{th} or 5^{th} percentile of intensities measured of the binding between the IgA and the plurality of glycans. In one embodiment, the at least one glycan or the at least two glycans are selected from the group consisting of:

### GlcNAcβ1-6Galβ1-4GlcNAc;

### GlcA;

### Galβ1-4(Fucα1-3)(6S)Glc; and

### Manα1-6(Manα1-3)Manα1-6(Manα1-3)Man.

In certain embodiments, the glycan-binding pattern comprises a glycan motif found in at least one glycan associated with colon cancer or a predisposition thereto. In one embodiment, the glycan motif is one or more of the following:

### Galα1-3Galβ1-4GlcNAc;

### Galα1-3Galβ1-4(Fuc); and Galα1-3Galβ1-4(Fucα1-3)GlcNAc.

In some embodiments, the at least one glycan is pre-determined to be bound at the highest intensity by the IgA in GLF from colon cancer patients. In some embodiments, the at least two glycans are pre-determined to be bound at the highest and the second highest intensities by the IgA in GLF from colon cancer patients. In one embodiment, the at least one glycan or the at least two glycans have an intensity of higher than 10 RFU. In one embodiment, the at least one glycan or the at least two glycans are at the 90^{th} percentile of all intensities measured of the binding between the IgA and the plurality of glycans. In one embodiment, the at least one glycan or the at least two glycans are selected from the group consisting of:

### Galα1-3Galβ1-4(Fucα1-3)GlcNAc;

### Galα1-3Galβ1-4GlcNAcβ1-2Manα1-6(Galα1-3Galβ1-4GlcNAcβ1-2Manα1-3)Manβ1-4GlcNAcβ1-4GlcNAc;

### Galα1-3Galβ1-4(Fucα1-3)GlcNAcβ1-2(Galα1-3Galβ1-4(Fucα1-3)GlcNAcβ1-2GalNAc;

### Galα1-3Galβ1-4GlcNAcβ1-2Manα1-6(Galα1-3Galβ1-4GlcNAcβ1-2Manα1-3)Manβ1-4GlcNAcβ1-4GlcNAc;

### Galα1-3Galβ1-4(Fucα1-3)GalNAc;

### Galα1-3Galβ1-4(Fucα1-3)GlcNAcβ1-2Manα1-6(Galα1-3Galβ1-4(Fucα1-3)GlcNAc; and

### Galα1-3Galβ1-4(Fucα1-3)GlcNAc.

In certain embodiments, the glycan-binding pattern comprises a glycan motif found in at least one glycan associated with colon adenoma or a predisposition thereto. In one embodiment, the glycan motif is one or more of the following:

### Galβ1-4GlcNAcβ1-3Galβ1-4GlcNAc; and

### Galβ1-4GlcNAc.

In some embodiments, the at least one glycan is pre-determined to be bound at the highest intensity by the IgA in GLF from colon adenoma patients. In some embodiments, the at least two glycans are pre-determined to be bound at the highest and the second highest intensities by the IgA in GLF from colon cancer patients. In one embodiment, the at least one glycan or the at least two glycans have an intensity of higher than 10 RFU. In one embodiment, the at least one glycan or the at least two glycans are at the 90^{th} percentile of all intensities measured of the binding between the IgA and the plurality of glycans. In one embodiment, the at least one glycan or the at least two glycans are selected from the group consisting of:

### Fucα1-2Galβ1-4(Fucα1-3)GlcNAcβ1-2Manα1-6(Fucα1-2Galβ1-4(Fucα1-3)GlcNAcβ1-2Manα1-3)Manβ1-4GlcNAcβ1-4GlcNAc;

### Galβ1-4GlcNAcβ1-6(Galβ1-4GlcNAcβ1-2)Manα1-6(Galβ1-4GlcNAcβ1-2Manα1-3)Manβ1-4GlcNAcβ1-4GlcNAc;

### Galβ1-4GlcNAcβ1-3Galβ1-4GlcNAcβ1-3Galβ1-4GlcNAcβ1-2Manα1-6(Galβ1-4GlcNAcβ1-3Galβ1-4GlcNAcβ1-3Galβ1-4GlcNAcβ1-2Manα1-3)Manβ1-4GlcNAcβ1-4GlcNAc;

Galβ1-4GlcNAcβ1-3Galβ1-4GlcNAcβ1-3Galβ1-4GlcNAcβ1-3Galβ1-4GlcNAcβ1-2Manα1-6(Galβ1-4GlcNAcβ1-3Galβ1-4GlcNAcβ1-3Galβ1-4GlcNAcβ1-3Galβ1-4GlcNAcβ1-2Manα1-3)Manβ1-4GlcNAcβ1-4GlcNAc; and

### Galβ1-4(Fucα1-3)GlcNAc.

In some embodiments, the glycan-binding pattern comprises at least one glycan associated with a GI tract cancer at a statistical significance testing p-value of ≤1.00e-6. In some embodiments, the glycan-binding pattern comprises at least one glycan having an area under curve (AUC) value of a receiver operating characteristic (ROC) curve of at least 0.95.

In certain embodiments, the plurality of glycans are exogenous glycans. In one embodiment, the plurality of glycans does not include an endogenous carbohydrate cancer antigen. In one embodiment, the plurality of glycans does not include the carbohydrate cancer antigen CA19-9.

In some embodiments, the method of the invention involves administering a lavage composition to the subject to thereby induce purgation of the subject's gastrointestinal tract. In some embodiments, the method of the invention involves administering a lavage composition to the subject to thereby cleanse the subject's gastrointestinal tract. In one embodiment, the lavage composition is administered orally. In one embodiment, the lavage composition is a solution or a solid composition that is reconstituted in a solvent prior to being administered to the subject.

In certain embodiments, the lavage composition includes an active ingredient selected from group consisting of polyethylene glycol (PEG), magnesium sulfate, sodium sulfate, potassium sulfate, magnesium citrate, sodium citrate, potassium citrate, ascorbic acid, citric acid, sodium picosulfate, sodium chloride, potassium chloride, bisacodyl, magnesium oxide, magnesium phosphate, potassium phosphate, sodium phosphate, monobasic sodium phosphate, dibasic sodium phosphate, potassium hydrogen tartrate, magnesium carbonate, magnesium hydroxide, sodium hydroxide, potassium hydroxide, sodium carbonate, sodium bicarbonate, ammonium carbonate, an anhydrous form thereof, a hydrate thereof, or a combination thereof.

In some embodiments, the lavage composition includes:

a PEG having a molecular weight of between about 3000 and about 4000, sodium sulfate, sodium bicarbonate, sodium chloride, and potassium chloride; or
a PEG having a molecular weight of between about 3000 and about 4000, sodium bicarbonate, sodium chloride, and potassium chloride; or
sodium sulfate, magnesium sulfate, and potassium sulfate; or
monobasic sodium phosphate and dibasic sodium phosphate, or a hydrate thereof.

In some embodiments, the method of the invention includes separating the GLF from the sample. In some embodiments, the GLF does not include solid or semisolid fecal matter, fecal proteins, and fecal immunoglobulins. In some embodiments, the method involves removing solid or semisolid fecal matter, fecal proteins and fecal immunoglobulins.

In some embodiments, the binding between the IgA and the plurality of glycans is detected and measured using an immunoassay, a chemiluminescent assay, a fluorescent assay, a colorimetric assay, or a mass spectrometry assay.

In another further aspect, the present disclosure relates to a method for detecting a glycan-binding pattern associated with a gastrointestinal (GI) tract cancer or predisposition thereto in a subject, or evaluating or monitoring the progression of GI tract cancer or the efficacy of GI cancer treatment by: obtaining a sample including gastrointestinal lavage fluid (GLF) from the subject, wherein the GLF includes immunoglobulin A (IgA); contacting the GLF with a plurality of glycans; and detecting binding between the IgA and the plurality of glycans, thereby detecting the glycan-binding pattern.

All embodiments of the method for detecting a glycan-binding pattern described above are as set forth above for the method for diagnosing a GI tract cancer or predisposition thereto in a subject.

In yet another further aspect, the present disclosure relates to a method for identifying a glycan associated with a gastrointestinal (GI) tract cancer, by: obtaining a sample including gastrointestinal lavage fluid (GLF) from at least one subject suffering from a GI tract cancer, wherein the GLF includes immunoglobulin A (IgA); contacting the GLF with a plurality of glycans; and detecting binding between the IgA and the plurality of glycans, thereby identifying the glycan.

In some embodiments, the GI tract cancer is pancreatic cancer. In one embodiment, the GI tract cancer is pancreatic ductal adenocarcinoma (PDAC). In one embodiment, the glycan comprises the glycan motif GlcNAcα1-4Galβ1-4GlcNAc.

In another embodiment, the GI tract cancer is colon cancer. In one embodiment, the glycan includes a glycan motif, for example, one or more of the following:

### Galα1-3Galβ1-4GlcNAc;

### Galα1-3Galβ1-4(Fuc); and

### Galα1-3Galβ1-4(Fucα1-3)GlcNAc.

In another embodiment, the GI tract cancer is colon adenoma. In one embodiment, the glycan includes a glycan motif, for example, one or more of the following:

### Galβ1-4GlcNAcβ1-3Galβ1-4GlcNAc; and

### Galβ1-4GlcNAc.

In some embodiments, the glycan-binding pattern includes at least one glycan associated with the GI cancer at a statistical significance testing p-value of ≤1.00e-6. In some embodiments, the glycan-binding pattern includes at least one glycan having an area under curve (AUC) value of a receiver operating characteristic (ROC) curve of at least 0.95.

In certain embodiments, the plurality of glycans are exogenous glycans. In one embodiment, the plurality of glycans does not include an endogenous carbohydrate cancer antigen. In one embodiment, the plurality of glycans does not include the carbohydrate cancer antigen CA19-9.

In some embodiments, the method of the invention includes administering a lavage composition to the subject to thereby induce purgation of the subject's gastrointestinal tract. In some embodiments, the method of the invention comprises administering a lavage composition to the subject to thereby cleanse the subject's gastrointestinal tract. In one embodiment, the lavage composition is administered orally. In one embodiment, the lavage composition is a solution or a solid composition that is reconstituted in a solvent prior to being administered to the subject.

In certain embodiments, the lavage composition includes an active ingredient selected from group consisting of polyethylene glycol (PEG), magnesium sulfate, sodium sulfate, potassium sulfate, magnesium citrate, sodium citrate, potassium citrate, ascorbic acid, citric acid, sodium picosulfate, sodium chloride, potassium chloride, bisacodyl, magnesium oxide, magnesium phosphate, potassium phosphate, sodium phosphate, monobasic sodium phosphate, dibasic sodium phosphate, potassium hydrogen tartrate, magnesium carbonate, magnesium hydroxide, sodium hydroxide, potassium hydroxide, sodium carbonate, sodium bicarbonate, ammonium carbonate, an anhydrous form thereof, a hydrate thereof, or a combination thereof.

In some embodiments, the lavage composition includes:

a PEG having a molecular weight of between about 3000 and about 4000, sodium sulfate, sodium bicarbonate, sodium chloride, and potassium chloride; or
a PEG having a molecular weight of between about 3000 and about 4000, sodium bicarbonate, sodium chloride, and potassium chloride; or
sodium sulfate, magnesium sulfate, and potassium sulfate; or
monobasic sodium phosphate and dibasic sodium phosphate, or a hydrate thereof.

In some embodiments, the method of the invention further involves separating the GLF from the sample. In some embodiments, the GLF does not comprise solid or semisolid fecal matter, fecal proteins, and fecal immunoglobulins. In some embodiments, the method involves removing solid or semisolid fecal matter, fecal proteins and fecal immunoglobulins.

In some embodiments, the binding between the IgA and the plurality of glycans is detected and measured using an immunoassay, a chemiluminescent assay, a fluorescent assay, a colorimetric assay, or a mass spectrometry assay.

In some embodiments, the method for identifying a glycan further includes: obtaining a sample including gastrointestinal lavage fluid (GLF) from at least one control subject, wherein the GLF from the control subject includes immunoglobulin A (IgA); contacting the GLF from the at least one control subject with a plurality of glycans; and detecting binding between the IgA and the plurality of glycans, identifying at least one glycan which binds at significantly different levels in GLF from the at least one subject with GI tract cancer as compared to the GLF from the at least one control subject. In some embodiments, the method for identifying a glycan includes obtaining samples comprising GLF from a plurality of control subjects.

In some embodiments, the method for identifying a glycan includes obtaining samples comprising GLF from a plurality of subjects suffering from a GI tract cancer.

### BRIEF DESCRIPTION OF THE DRAWINGS

FIG. 1 depicts four pie charts that each show that IgA represents a large percentage of proteins in the gastrointestinal lavage fluid (GLF) collected from healthy subjects, as well as PDAC, colon adenoma and colon cancer patients. The percentage of IgA in each chart is shown relative to the top 20 proteins identified in each sample group as determined by mass spectrometry. Each pie chart represents the composite spectral counting results from each of the conditions examined in the studies described herein. These data show that IgA is highly abundant across all conditions examined.

FIG. 2 depicts 20 bar graphs that each show the IgA composition for 20 samples that were used to screen against a glycan array, determined using spectral counting and LC-MS/MS data. This information shows that IgA often makes up a large portion of the protein content of GLF, and that the overall IgA levels do not significantly fluctuate between conditions (healthy subjects and PDAC, colon adenoma and colon cancer patients).

FIG. 3A depicts the glycan binding profile of IgA in GLF samples collected from pancreatic cancer patients in comparison with the combination of all other non-PDAC conditions studied (combination of healthy subjects, colon adenoma patients, colon cancer patients).

FIG. 3B is a close-up view of a particular region of interest where IgA from PDAC patients binds certain glycans (i.e. Glycan ID NOs: 337 to 343) at very high intensities.

FIG. 3C depicts the glycan binding profile of IgA in GLF samples collected from pancreatic cancer patients in comparison with healthy, colon adenoma, and colon cancer patients.

FIG. 4A is a heat map demonstrating differential glycan interactions by healthy subjects and pancreatic cancer patients, colon adenoma patients and colon cancer patients, where reactivity to selected glycans is presented from each patient sample.

FIG. 4B lists the four most statistically significant glycans where IgA reactivity was increased in PDAC patients versus non-PDAC patients.

FIG. 5A depicts the glycans where the IgA in GLF samples collected from healthy subjects and patients of pancreatic cancer bind at the highest binding intensities, with the related motifs of these glycans outlined within the boxes with dashed lines.

FIG. 5B depicts the glycans where the IgA in GLF samples collected from colon adenoma patients and colon cancer patients bind at the highest binding intensities, with the shared motifs of these glycans outlined within the boxes with dashed lines.

FIG. 5C depicts the glycans where the IgA in GLF samples collected from healthy subjects and pancreatic cancer patients bind at the lowest binding intensities, with the shared motifs of these glycans outlined within the boxes with dashed lines.

FIG. 5D depicts the glycans where the IgA in GLF samples collected from colon adenoma patients and colon cancer patients bind at the lowest binding intensities, with the related motifs of these glycans outlined within the boxes with dashed lines.

FIG. 6 is a receiver operating characteristic (ROC) curve showing sensitivity and specificity of reactivity for Glycan ID NO.: 37.

### DETAILED DESCRIPTION OF THE INVENTION

The present disclosure is based, at least in part, on the unexpected discovery that the immunoglobulins found in gastrointestinal lavage fluid (GLF), in particular, immunoglobulin A (IgA), can reveal disease-specific glycan binding patterns, for example, when the GLF containing the immunoglobulins are screened against a glycan microarray, particularly diseases associated with the gastrointestinal (GI) tract and, in particular, GI tract cancers. This discovery is significant, especially considering the state of the art where carbohydrate or glycan binding by antibodies is so much more poorly understood as compared to peptide-targeting by antibodies.

In these aspects, screening immunoglobulins from a subject's GLF against a plurality of exogenous glycans, as proposed in the present disclosure, is clearly advantageous over existing techniques, such as the use of CA19-9 as a pancreatic cancer biomarker, because the methods of the invention utilize a sample that is more specific or localized to the organ(s) of interest. Moreover, the methods of the invention provide higher specificity, therefore, allowing for improved ability to distinguish between different types of GI tract cancers.

This approach that capitalizes on the highly specific and highly sensitive characteristics of the body's own immune system provides a new and unique approach to allow for the early, specific and reliable detection of pancreatic cancer (for example, PDAC), colon cancer, colon adenoma and other GI tract cancers.

### Definitions

Unless otherwise defined herein, scientific and technical terms used in connection with the present invention shall have the meanings that are commonly understood by those of ordinary skill in the art. The meaning and scope of the terms should be clear. However, in the event of any latent ambiguity, definitions provided herein take precedent over any dictionary or extrinsic definition. Further, unless otherwise required by context, singular terms, for example, those characterized by “a” or “an”, shall include pluralities. In this application, the use of “or” means “and/or”, unless stated otherwise.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches. Typically, as used herein, the term “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human.

The term “control subject” or “healthy subject” as used herein, refers to a healthy subject not afflicted with pancreatic cancer and/or a GI tract cancer, for example, colon cancer. The term “control sample,” as used herein, refers to any clinically relevant control sample, including, for example, a GLF sample from a healthy subject not afflicted with pancreatic cancer and/or a GI tract cancer, for example colon cancer; a GLF sample from a subject having a less severe or slower progressing pancreatic cancer and/or a GI tract cancer, for example, colon cancer, than the subject to be assessed; a GLF sample from a subject having some other type of cancer or disease, and the like. A control sample may include a GLF sample derived from one or more subjects. A control sample may also be a GLF sample made at an earlier timepoint from the subject to be assessed. For example, the control sample could be a GLF sample taken from the subject to be assessed before the onset of the pancreatic cancer and/or a GI tract cancer, for example colon cancer, at an earlier stage of disease, or before the administration of treatment or of a portion of treatment. The control sample may also be a GLF sample from an animal model, or from a tissue or cell lines derived from the animal model, of the pancreatic cancer and/or a GI tract cancer, for example, colon cancer.

In particular embodiments, the glycan binding pattern is compared to a control level. The term “control level” refers to an accepted or pre-determined binding of the at least one glycan or glycan motif to IgA which is used to compare with the level of the at least one glycan or glycan motif in the GLF sample derived from the subject being tested. In one embodiment, the control level is based on the level of binding of the at least one glycan or glycan motif to IgA in a GLF sample(s) from a subject(s) having slow disease progression. In another embodiment, the control level of FRα not bound to a cell is based on the level of binding of the at least one glycan or glycan motif to IgA in a GLF from a subject(s) having rapid disease progression. In another embodiment, the control level of FRα is based on level of binding of the at least one glycan or glycan motif to IgA in a GLF sample(s) from an unaffected, i.e., non-diseased, subject(s), i.e., a subject who does not have pancreatic cancer or GI tract cancer. In yet another embodiment, the control level of FRα is based on the level of binding of the at least one glycan or glycan motif to IgA in a GLF sample(s) from a subject(s) prior to the administration of a therapy for pancreatic cancer or GI tract cancer. In another embodiment, the control level of FRα is based on the level of binding of the at least one glycan or glycan motif to IgA in a GLF sample(s) from a subject(s) having pancreatic or GI tract cancer that is not contacted with a test compound. In another embodiment, the control level of FRα is based on the level of binding of the at least one glycan or glycan motif to IgA in a GLF sample(s) from a subject(s) not having pancreatic or GI tract cancer that is contacted with a test compound.

In one embodiment, the control is a standardized control, such as, for example, a control which is predetermined using an average of level of at least one glycan or glycan motif binding to immunoglobulin A from GLF samples from healthy subjects.

As used herein, a “patient” refers to a subject that has been diagnosed by a medical practitioner as having pancreatic cancer or any other gastrointestinal tract cancer or predisposition to the same. The term can further refer to a population of patients suffering from pancreatic cancer or any other gastrointestinal tract cancer, from whom a GLF sample may be obtained, for example, upon which a reference glycan-binding pattern or a reference level of a glycan-binding pattern can be pre-determined.

The terms “cancer” or “tumor” are well-known in the art and refer to the presence, e.g., in a subject, of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within a subject, or may be non-tumorigenic cancer cells, such as leukemia cells. As used herein, the term “cancer” includes pre-malignant as well as malignant cancers.

As used herein, the term “predisposition” refers to liability or tendency to super from a particular medical condition, which includes genetic and lifestyle factors.

As used herein, “gastrointestinal tract” or “GI tract” is an organ system within humans and other animals which takes in food, digests it to extract and absorb energy and nutrients, and expels the remaining waste as feces. The GI tract includes all structures between the mouth and the anus, forming a continuous passageway that includes the main organs of digestion, namely, the stomach, small intestine (including duodenum, jejunum, ileum), and large intestine (including cecum, colon, rectum, anal canal). As used herein, the GI tract also refers to organs of digestion (the tongue, salivary glands, pancreas, liver, gallbladder, biliary tract). The tract may also be divided into foregut, midgut, and hindgut, reflecting the embryological origin of each segment.

As used herein, a “gastrointestinal tract cancer” or “GI tract cancer” refers to cancer of any GI tract organ as defined above, preferably cancer of the stomach (or gastric cancer), liver (including hepatocellular carcinoma), pancreas (including pancreatic duct adenocarcinoma), esophagus, colon (including colon cancer, colon adenoma, colorectal cancer), rectum, biliary tract or system, small intestine, and large intestine.

As used herein, “pancreas” refers to the art recognized organ. Pancreas refers to the organ which constitutes a collection of a plurality of cell types held together by connective tissue, such that the plurality of cells includes but are not limited to acini calls, ductal cells and islet cells. The “acini” produce many of the enzymes, such as lipase, which are needed to digest food in the duodenum. The enzymes produced by the acini are carried to the duodenum by small channels called ducts. Typically, ductal cells are held in place by connective tissue in close proximity to vascular cells and nerve cells. Islets of Langerhans are typically embedded between exocrine acini units of the pancreas. Examples of islet endocrine cells are Alpha cells that secrete glucagon which counters the action of insulin while Beta cells secrete insulin, which helps control carbohydrate metabolism.

As used herein, the term “pancreatic cancer” refers to the art recognized disease and includes cancers that originate in the tissue that comprises a pancreas. In various embodiments, the pancreatic cancer is an exocrine pancreatic cancer, a pancreatic cystic neoplasm or a pancreatic endocrine tumor.

In a particular embodiment, the pancreatic cancer is an exocrine pancreatic cancer selected from the group consisting of pancreatic ductal adenocarcinoma (PDAC), adenosquamous carcinoma, squamous cell carcinoma, giant cell carcinoma, acinar cell carcinoma and small cell carcinoma.

In a particular embodiment, the pancreatic cancer is pancreatic ductal adenocarcinoma (PDAC), e.g., resectable pancreatic ductal adenocarcinoma (PDAC), which arises within the exocrine component of the pancreas. As used herein, “adenocarcinoma” refers to a cancerous tumor as opposed to an “adenoma” which refers to a benign (non-cancerous) tumor made up of cells that form glands (collections of cells surrounding an empty space). As used herein, “pancreatic ductal adenocarcinoma cell” refers to a cancerous cell that has the capability to form or originated from the ductal lining of the pancreas. A pancreatic ductal adenocarcinoma cell may be found within the pancreas forming a gland, or found within any organ as a metastasized cell or found within the blood stream of lymphatic system. As used herein, “ductal cell”, in reference to a pancreas, refers to any cell that forms or has the capability to form or originated from the ductal lining of ducts within and exiting from the pancreas.

In another embodiment, the pancreatic cancer is a pancreatic endocrine tumor, also known as islet cell tumors, pancreas endocrine tumors (PETs) and pancreatic neuroendocrine tumors (PNETs), which arises from islet cells. In a particular embodiment, the pancreatic cancer is an endocrine pancreatic cancer selected from the group consisting of insulinomas (i.e., arising from insulin-producing cells), glucagonomas (i.e., arising from glucagon-producing cells), somatostatinomas (i.e., arising from somatostatin-making cells), gastrinomas (i.e., arising from a gastrin-producing cells), VlPomas (arising from vasoactive intestinal peptide-making cells) and non-secreting islet tumors of the pancreas.

As used herein, the terms “immunoglobulin” (Ig) and “antibody” (Ab) refer to a class of large, Y-shaped polypeptide molecules produced by mainly plasma cells and is part of the immune system, or any peptide fragment or an antigen-binding fragment thereof. Immunoglobulins recognize and specifically bind to a target antigen such as a target antigen of a pathogen or a cancer cell, such as a protein, polypeptide, peptide, carbohydrate, glycan, polysaccharide, polynucleotide, lipid, or combinations of the foregoing through at least one antigen recognition site within the variable region of the immunoglobulin molecule. Immunoglobulins can be of any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations.

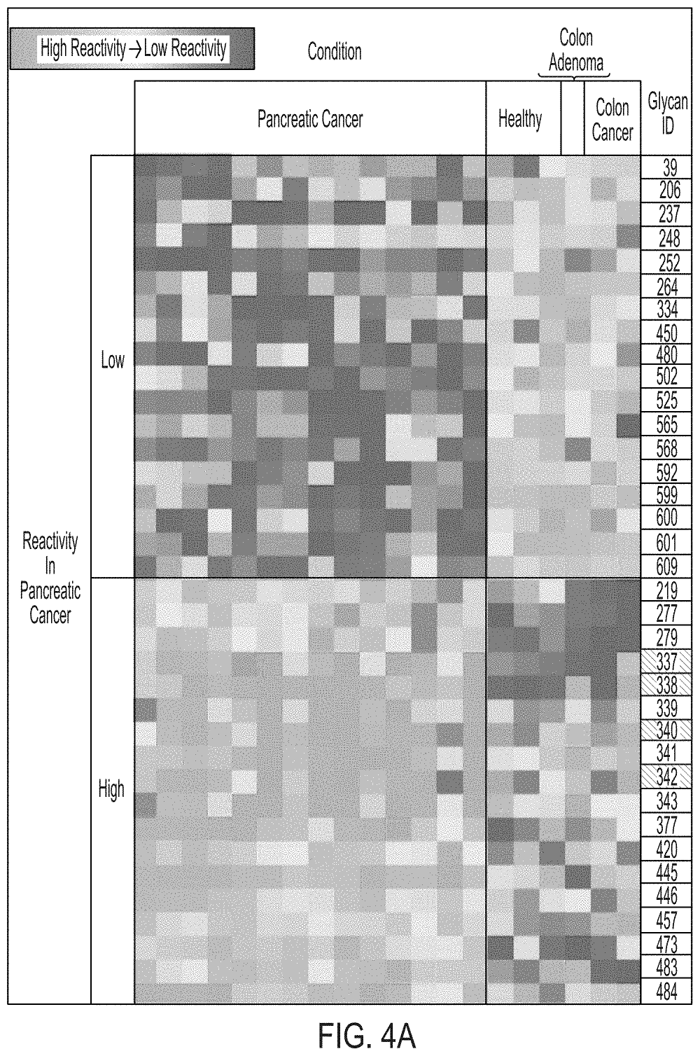

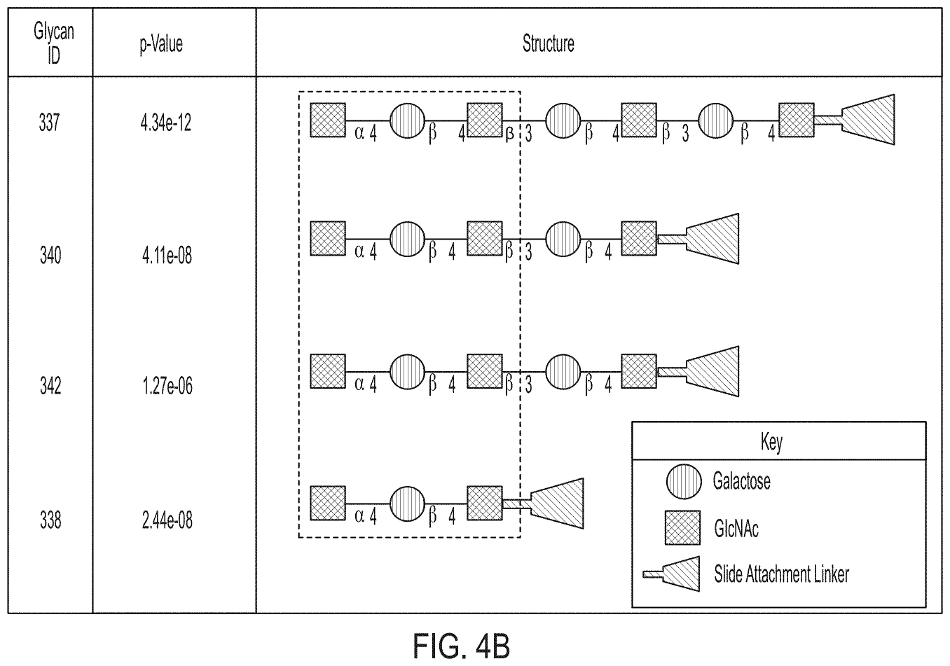

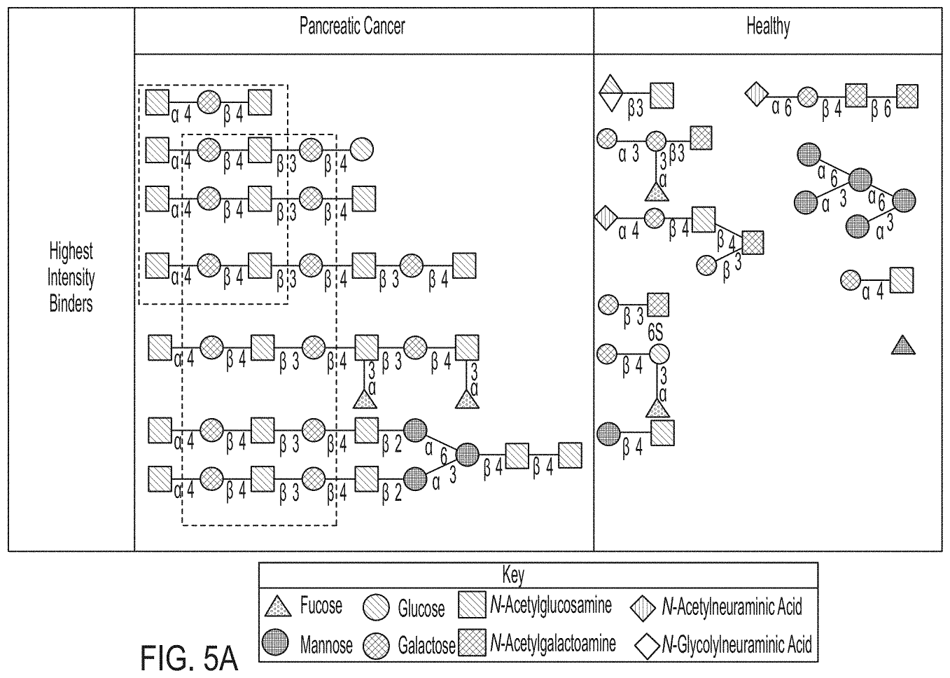

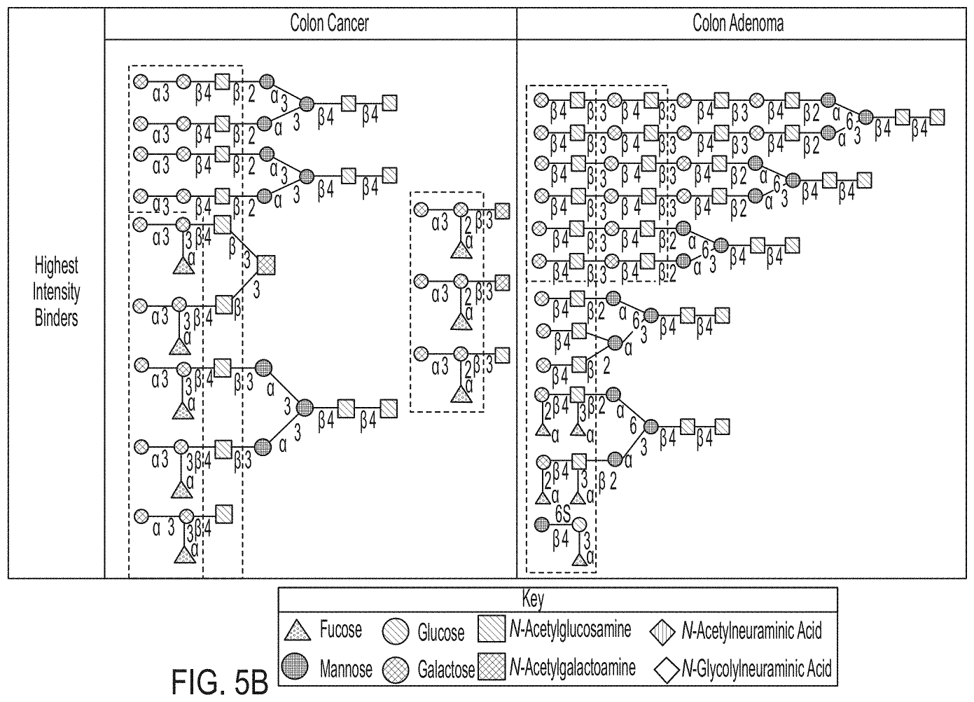

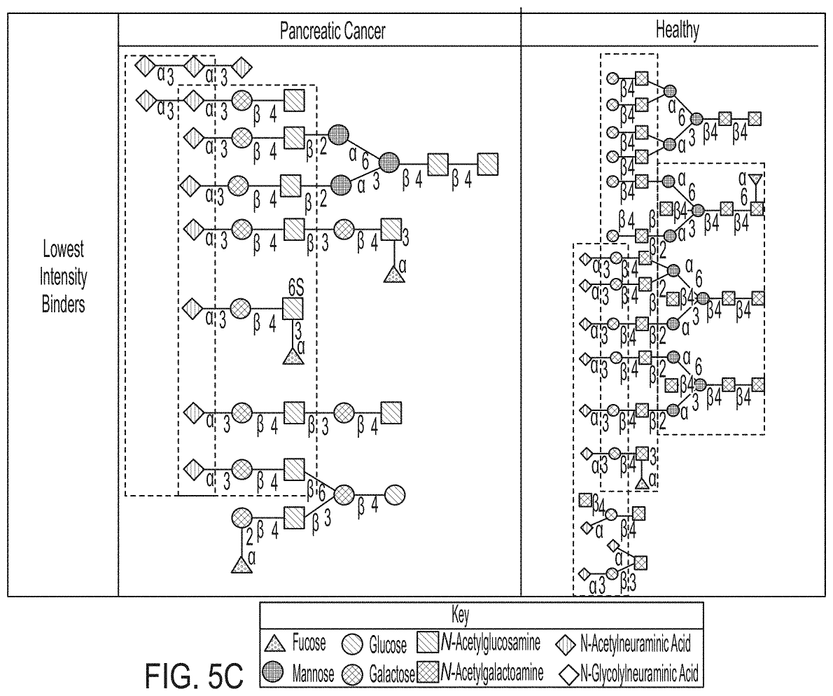

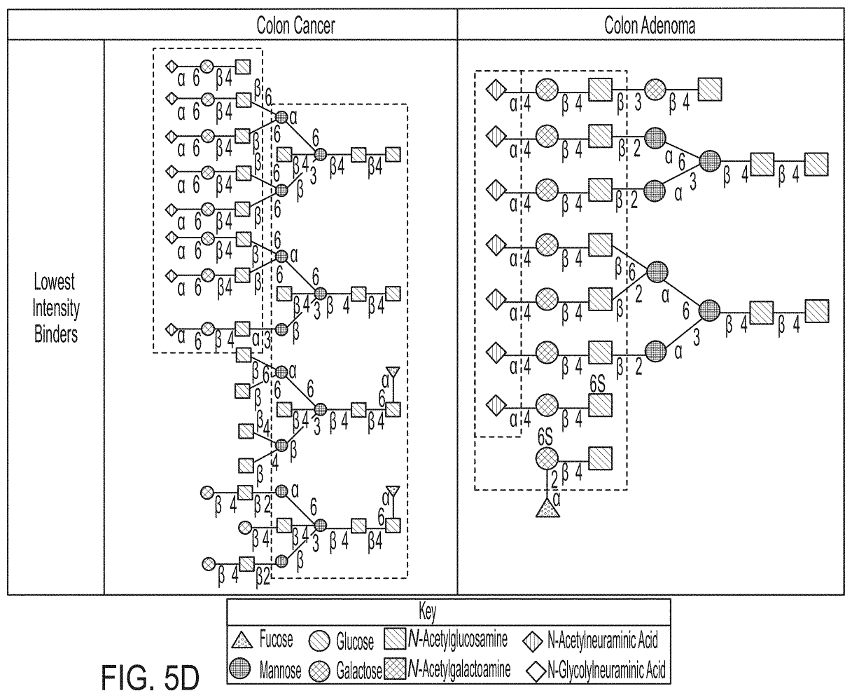

As used herein, the terms “glycan” and “polysaccharide” are defined by International Union of Pure and Applied Chemistry (IUPAC) as synonyms meaning compounds consisting of a large number of linear or branched monosaccharides linked glycosidically or via glycosidic bonds. However, in practice and as used herein the term “glycan” is extended to refer to the carbohydrate portion of a glycoconjugate, such as a glycoprotein, glycolipid, or a proteoglycan, even if the carbohydrate is only an oligosaccharide. One distinguishes between α- and β-glycosidic bonds based on the relative stereochemistry (R or S) of the anomeric position and the stereocenter furthest from C1 in the saccharide. An α-glycosidic bond is formed when both carbons have the same stereochemistry, whereas a β-glycosidic bond occurs when the two carbons have different stereochemistry. As used herein, the “α” and “β” denoting the relative stereochemistry of the glycosidic bonds as explained are also respectively expressed as “a” and “b” as an alternative (see e.g., Table 1). Consistent with what is practiced in the art by glycan scientists globally the glycans are expressed or defined herein in a three-dimensional Symbol Nomenclature for Glycans (SNFG) format (see e.g., FIGS. 4B, 5A, 5B) or in a IUPAC condensed linear text nomenclature (see e.g., Table 1, the claims and throughout the text of the specification), including additional terminal linker or spacer structures (see e.g. Table 2), that do not materially alter the ability of the glycan to be recognized and bound by immunoglobulins. Monosaccharides can be further categorized as hexose, pentose, tetrose, heptose, deoxysugar, di-deoxysugar, amino sugar, uronic acid, sialic acid, etc.). Common monosaccharides and their codes include but are not limited to D-glucose (Glc), D-galactose (Gal), L-galactose (LGal), D-mannose (Man), D-fucose (Fuc), L-fucose (LFuc), N-acetyl-D-glucosamine (GlcNac), N-acetyl-D-galactosamine (GalNAc), neuraminic acid (Neu), N-acetylneuraminic acid (NeuSAc), D-glucoronic acid (GlcA), D-galacturonic acid (GalA), D-mannuronic acid (ManA), etc. The numbers shown in the glycan structures, whether using the symbol or text nomenclatures, denote at which carbon atom in the monosaccharide that a glycosidic bond is formed.

As used herein, an “exogenous glycan” refers to a glycan that is not produced inside the body, for example a glycan in a glycan array. An “endogenous glycan” refers to a glycan that is produced inside the body, such as glycans attached to cell surface and cell-surface antigens.

An “endogenous carbohydrate cancer antigen”, such as the carbohydrate cancer antigen CA19-9, is a glycan that can serve as cancer or tumor marker, which includes a glycan expressed on a cancer or tumor cell.

As used herein, a “glycan motif” refers to a single monosaccharide, a disaccharide structure or sequence, or a short oligosaccharide structure or sequence (e.g., comprising 3, 4, 5, 6, 7, 8, 9 or 10 monosaccharides linked glycosidically) that is within a glycan or polysaccharide that is highly conserved among at least a plurality of glycans, e.g., at least two, at least three, at least four, at least five, at least six, at least seven, at least eight, at least nine, or at least ten glycans. In some embodiments, apart from the shared glycan motif, these glycans share another commonality in that they are bound by the immunoglobulins found in the GLF of a pancreatic cancer or GI tract cancer patient, such as IgA, at the highest intensities or the lowest intensities among all glycans tested or screened in the methods of the invention.

As used herein, a “glycan-binding pattern” or a “glycan-binding profile” refers to the binding of at least one glycan or a specific subset of glycans that the GLF immunoglobulins (e.g., IgA) are subjected to, wherein the glycans are associated with a specific medical condition (e.g., pancreatic cancer or GI tract cancer) or predisposition thereto, for example, wherein the binding intensities of this specific subset of glycans are pre-determined to be the highest and are unique to the specific medical condition or predisposition to the same. The identification and pre-determination of this specific subset of glycans may be carried out, for example, using the methods and techniques described in Examples 3 and 4, but are not so limited. In other words, as used herein, a “glycan-binding pattern” is indicative of a specific medical condition or predisposition to the same, such as pancreatic cancer or a gastrointestinal (GI) tract cancer, for example, colon cancer, as defined herein, or other non-cancer diseases of the GI tract. In some embodiments, the glycan-binding pattern serves as a disease-specific immunosignature, where the glycan binding of the GLF immunoglobulins (e.g., IgA) of a patient of a specific disease is different from that of a healthy subject and a patient or another disease due to a unique and disease-specific immune response to the disease (e.g., tumor, cancer cells). Alternatively or in combination, a glycan-binding pattern includes at least one glycan or specific subset of glycans that the GLF immunoglobulins (e.g., IgA) are subjected to, wherein the binding intensities of this specific subset of glycans are associated to the specific medical condition or predisposition to the same, for example, by binding at the lowest intensities. In certain embodiments, a glycan-binding pattern further includes a glycan motif that is associated with a specific medical condition (e.g., pancreatic cancer, colon cancer or other GI tract cancer) or predisposition thereto, for example, wherein the glycan motif is observed among at least some of those glycans having the highest binding intensities and/or at least some of those glycans having the lowest binding intensities.

As used herein, a “lavage composition” is a composition having one or more active ingredients that can induce purgation of a subject's gastrointestinal tract. Such gastrointestinal lavage compositions are used as a lower gastrointestinal tract preparation for sigmoidoscopy, colonoscopy, radiographic examination, preparation for patients undergoing bowel or colorectal surgery. In some embodiments, the lavage composition can induce the purgation to thereby allow for the collection of a sample comprising gastrointestinal lavage fluid (GLF) or a GLF sample. The various embodiments of a lavage compositions are separately discussed herein.

Accordingly, as used herein, a “sample comprising/containing gastrointestinal lavage fluid”, or “sample comprising/containing GLF”, or “a gastrointestinal lavage fluid sample”, or “a GLF sample” refers to a sample that is predominantly in fluid form or liquid form that is excreted from the body of a subject via the gastrointestinal tract, as a result of having his or her gastrointestinal tract purged or cleansed via being administered the lavage composition.

As used herein, the term “gastrointestinal lavage fluid” or “GLF” refers to a usually large volume of fluid or liquid (e.g., more than 10 mL, preferably more than 20 mL, more than 50 mL, more than 100 mL) that is found in a GLF sample, which is distinct from solid or semisolid fecal matter or any other rectal effluent not only in terms of the physical form (i.e., liquid vs. solid or semisolid), but also in terms of the source of the substances in question. Specifically, the source of GLF is the lavage composition that is administered to the subject, whereas the source of fecal matter or rectal effluent is food ingested by the subject. A colonic lavage fluid, in accordance with the present disclosure, is synonymous with GLF.

GLF is rich in immunoglobulins (particularly IgA) and other proteins that are separate and distinct from fecal proteins and immunoglobulins. When a GLF sample is initially obtained from a subject, GLF including its proteins and immunoglobulins may be separated from fecal matter, fecal proteins and fecal proteins, for example, by centrifugation of the crude GLF sample at a relatively very high speed (e.g., more than 10,000 g, more than 15,000 g, more than 20,000 g, more than 30,000 g), and optionally for a relatively brief duration (e.g., no more than 10 min, no more than 5 min).

GLF also largely maintains the chemical composition of the lavage composition administered to the subject, which may include polyethylene glycol (PEG), electrolytes, pH balancers, salts, etc. These substances help to protect the GLF proteins and immunoglobulins against damage and denaturation, therefore preserving their structural and functional properties as much as possible.

Methods of obtaining a GLF sample are-well known in the art. For example, before, during or after medical and or diagnostic procedures such as sigmoidoscopy, colonoscopy, radiographic examination, preparation for patients undergoing bowel surgery, it is important that the bowels and colon be thoroughly purged and cleaned. In some embodiments, the GLF sample is self-collected by the subject. In addition to the protective substances that are already present in the GLF as described above, enzyme inhibitors and denaturants may be used to preserve the quality of the GLF. In some embodiments, the pH of the sample may be adjusted to help stabilize the samples.

### Methods of the Invention

Methods of the invention, in accordance with the present disclosure, include diagnostic and prognostic methods of pancreatic cancer, and diagnostic and prognostic methods of gastrointestinal (GI) tract cancers. Diagnostic and prognostic methods of other non-cancer disease of the pancreas and the GI tract are also contemplated herein.

### Pancreatic Cancer

### Diagnostic Methods of Pancreatic Cancer

In a first aspect, the present disclosure relates to a method for diagnosing pancreatic cancer or predisposition thereto (i.e., assessing whether a subject is afflicted with pancreatic cancer or a predisposition thereto), by: (i) obtaining a sample including gastrointestinal lavage fluid (GLF) from the subject, wherein the GLF includes immunoglobulin A (IgA); (ii) contacting the GLF with a plurality of glycans; and (iii) detecting binding between the IgA and the plurality of glycans, thereby detecting a glycan-binding pattern; wherein the glycan-binding pattern is associated with pancreatic cancer or predisposition thereto.

In a second aspect, the present disclosure relates to a method for detecting a glycan-binding pattern associated with pancreatic cancer or predisposition thereto in a subject, by: (i) obtaining a sample including gastrointestinal lavage fluid (GLF) from the subject, wherein the GLF includes immunoglobulin A (IgA); (ii) contacting the GLF with a plurality of glycans; and (iii) detecting binding between the IgA and the plurality of glycans, thereby detecting the glycan-binding pattern.

### Prognostic Methods of Pancreatic Cancer

In a third aspect, the present disclosure relates to a method for evaluating or monitoring the progression of pancreatic cancer or the efficacy of a pancreatic cancer treatment via a glycan-binding pattern associated with pancreatic cancer or predisposition thereto in a subject, by: (i) obtaining a sample including gastrointestinal lavage fluid (GLF) from the subject, wherein the GLF includes immunoglobulin A (IgA); (ii) contacting the GLF with a plurality of glycans; and (iii) detecting binding between the IgA and the plurality of glycans, thereby detecting the glycan-binding pattern.

In some embodiments, in a method in accordance with any one of the first, second, or third aspects described above, or any one of the fourth, fifth, sixth or seventh aspects described below, including any one or more of the embodiments of these methods as described herein, the glycan-binding pattern includes at least one glycan associated with pancreatic cancer or predisposition thereto. In one embodiment, the glycan-binding pattern includes at least two glycans associated with pancreatic cancer or predisposition thereto. As used herein, the term “at least two” refers to two or more, preferably two, three, four, five, six, seven, eight, nine, or ten. As used herein, the term “at least one” refers to one or more, and encompasses “at least two” as defined herein. In one embodiment, the glycan-binding pattern comprises a glycan motif found in at least one glycan associated with pancreatic cancer or a predisposition thereto. In one embodiment, the glycan motif is GlcNAcα1-4Galβ1-4GlcNAc.

In some embodiments, in a method in accordance with any one of the first, second, or third aspects described above, or any one of the fourth, fifth, sixth or seventh aspects described below, including any one or more of the embodiments of these methods as described above, the at least one glycan is pre-determined to be bound at the highest intensity by the IgA in GLF from pancreatic cancer patients. In one embodiment, the at least two glycans are pre-determined to be bound at the highest and the second highest intensities by the IgA in GLF from pancreatic cancer patients. In one embodiment, the at least one glycan or the at least two glycans have an intensity of higher than 8, 9, 10 or 11 RFU. In a particular embodiment, the at least one glycan or the at least two glycans have an intensity of higher than 10 RFU. In one embodiment, the at least one glycan or the at least two glycans are at least the 75^{th}, 80^{th}, 85^{th}, 90^{th}, 91^{st}, 92^{nd}, 92^{nd}, 93^{rd}, 94^{th}, 95^{th}, 95^{th}, 96^{th}, 97^{th}, 97^{th}, 98^{th}, or 99^{th} percentile of all intensities measured of the binding between the IgA and the plurality of glycans and are associated with the pancreatic cancer, colon cancer or GI tract cancer. In one embodiment, the at least one glycan or at least two glycans pre-determined to be bound at the highest intensities by the IgA in GLF from pancreatic cancer patients is selected from the group consisting of:

### GlcNAcα1-4 Galβ1-4 GlcNAcβ1-3 Galβ1-4 GlcNAcβ1-3 Galβ1-4 GlcNAc;

### GlcNAcα1-4Galβ1-4GlcNAc;

### GlcNAcα1-4Galβ1-4GlcNAcβ1-3Galβ1-4Glc;

### GlcNAcα1-4Galβ1-4GlcNAcβ1-3Galβ1-4(Fucα1-3)GlcNAcβ1-3Galβ1-4(Fucα1-3)GlcNAc;

### GlcNAcα1-4Galβ1-4GlcNAcβ1-3Galβ1-4GlcNAc; and

### GlcNAcβ1-3Galβ1-4GlcNAcβ1-3Galβ1-4GlcNAcβ1-2Manα1-6(GlcNAcβ1-3Galβ1-4GlcNAcβ1-3Galβ1-4GlcNAcβ1-2Manα1-3)Manβ1-4GlcNAcβ1-4GlcNAc.

Additionally or alternatively, in certain embodiments, in a method in accordance with any one of the first, second, or third aspects described above, or any one of the fourth, fifth, sixth or seventh aspects described below, including any one or more of the embodiments of these methods as described above, the glycan-binding pattern further comprises at least one glycan pre-determined to be bound at the lowest intensity by the IgA in GLF from pancreatic cancer patients. In one embodiment, the at least one or the at least two glycans are pre-determined to be bound at the lowest and the second lowest intensities by the IgA in GLF from pancreatic cancer patients. In one embodiment, the at least one glycan or the at least two glycans have an intensity of no higher than 10 RFU, no higher than 9 RFU, no higher than 8 RFU, no higher than 7 RFU, or no higher than 6 RFU. In one embodiment, the at least one glycan or the at least two glycans are less than at least the 25^{th}, 20^{th}, 15^{th}, 10^{th}, 9^{th}, 8^{th}, 7^{th}, 6^{th}, 5^{th}, 4^{th}, 3^{rd}, 2^{nd}, or 1^{st} percentile, of all intensities measured of the binding between the IgA and the plurality of glycans. In one embodiment, the at least one glycan or at least two glycans pre-determined to be bound at the lowest intensities by the IgA in GLF from pancreatic cancer patients is selected from the group consisting of:

### GlcNAcβ1-6Galβ1-4GlcNAc;

### GlcA;

### Galβ1-4(Fucα1-3)(6S)Glc; and

### Manα1-6(Manα1-3)Manα1-6(Manα1-3)Man.

In some embodiments, in a method in accordance with any one of the first, second, or third aspects described above, or any one of the fourth, fifth, sixth or seventh aspects described below, including any one or more of the embodiments of these methods as described above, the glycan-binding pattern comprises at least one or at least two glycans each associated with pancreatic cancer or GI tract cancer, e.g., colon cancer, at a statistical significance testing p-value of ≤1.00e-6, e.g., 1.00e-6, 1.00e-7, ≤1.00e-7, 1.00e-8, or ≤1.00e-8.

In some embodiments, in a method in accordance with any one of the first, second, or third aspects described above, or any one of the fourth, fifth, sixth or seventh aspects described below, including any one or more of the embodiments of these methods as described herein, the glycan-binding pattern includes at least one or at least two glycans associated with pancreatic cancer or a GI tract cancer, e.g., colon cancer, and each having an area under curve (AUC) value of a receiver operating characteristic (ROC) curve of at least 0.95, e.g., 0.95, 0.96, 0.97, 0.98, or 0.99. As known in medical diagnosis, an AUC-ROC value of 1 is indicative of perfect diagnosis in terms of both sensitivity and specificity of a technique, a method, a tool, an instrument or a biomarker, etc.

In some embodiments, in a method in accordance with any one of the first, second, or third aspects described above, or any one of the fourth, fifth, sixth or seventh aspects described below, including any one or more of the embodiments of these methods as described herein, the IgA is IgA1. In another embodiment, the IgA is IgA2. In yet another embodiment, the IgA is a mixture of IgA1 and IgA2. In one embodiment, IgA represents at least 5%, at least 10%, at least 20%, at least 25%, at least 30%, at least 40%, at least 50%, at least 60%, at least 70%, at least 75%, at least 80%, or at least 90% of the total protein content in the GLF.

In some embodiments, in a method in accordance with any one of the first, second, or third aspects described above, or any one of the fourth, fifth, sixth or seventh aspects described herein, including any one or more of the embodiments of these methods as described above, the plurality of glycans are exogenous glycans. In one embodiment, the plurality of glycans does not include an endogenous cancer antigen. In one embodiment, the plurality of glycans does not include the cancer antigen CA19-9.

In some embodiments, in a method in accordance with any one of the first, second, or third aspects described above, including any one or more of the embodiments of these methods as described herein, the pancreatic cancer is any one selected from the list of pancreatic cancer subtypes listed above. In one embodiment, in a method in accordance with any one of the first, second or third aspects described above, including any one or more of the embodiments of these methods as described above, the pancreatic cancer is pancreatic ductal adenocarcinoma (PDAC).

In some embodiments, in a method in accordance with any one of the first, second, or third aspects described above, or any one of the fourth, fifth, sixth or seventh aspects described below, including any one or more of the embodiments of these methods as described above, the method further comprises administering a lavage composition to the subject to thereby induce purgation of the subject's gastrointestinal tract, and/or to thereby cleanse the subject's gastrointestinal tract. In one embodiment, the lavage composition is administered orally. In one embodiment, the lavage composition is a solution or a solid composition that is reconstituted in a solvent prior to being administered to the subject. In some embodiments, the lavage composition includes an active ingredient selected from the group consisting of polyethylene glycol (PEG), magnesium sulfate, sodium sulfate, potassium sulfate, magnesium citrate, sodium citrate, potassium citrate, ascorbic acid, citric acid, sodium picosulfate, sodium chloride, potassium chloride, bisacodyl, magnesium oxide, magnesium phosphate, potassium phosphate, sodium phosphate, monobasic sodium phosphate, dibasic sodium phosphate, potassium hydrogen tartrate, magnesium carbonate, magnesium hydroxide, sodium hydroxide, potassium hydroxide, sodium carbonate, sodium bicarbonate, ammonium carbonate, an anhydrous form thereof, a hydrate thereof, or a combination thereof. In further embodiments, the lavage composition comprises: a PEG having a molecular weight of between about 3000 and about 4000, sodium sulfate, sodium bicarbonate, sodium chloride, and potassium chloride; or a PEG having a molecular weight of between about 3000 and about 4000, sodium bicarbonate, sodium chloride, and potassium chloride; or sodium sulfate, magnesium sulfate, and potassium sulfate (e.g., SUPREP®); or monobasic sodium phosphate and dibasic sodium phosphate, or a hydrate thereof. Other lavage compositions suitable for oral administration and for use a method of the invention (i.e., a method in accordance with any one of the first, second, or third aspects described above, or any one of the fourth, fifth, sixth or seventh aspects as described below, including any one or more of the embodiments of these methods as described herein) are discussed in greater detail below.

In certain embodiments, in a method in accordance with any one of the first, second, or third aspects described above, or any one of the fourth, fifth, sixth or seventh aspects as described below, including any one or more of the embodiments of these methods as described herein, the method further includes separating the GLF from the sample. Techniques for the separation of the GLF from the GLF sample are described above.

In some embodiments, in a method in accordance with any one of the first, second, or third aspects described above, or any one of the fourth, fifth, sixth or seventh aspects as described below, including any one or more of the embodiments of these methods as described herein, the GLF does not include solid or semisolid fecal matter, fecal proteins, and fecal immunoglobulins. In some embodiment, the method further includes the step of removing the solid or semisolid fecal matter, fecal proteins, and fecal immunoglobulins from the sample.

In some embodiments, in a method in accordance with any one of the first, second, or third aspects described above, or any one of the fourth, fifth, sixth or seventh aspects as described below, including any one or more of the embodiments of these methods as described herein, the binding between the GLF IgA and the plurality of glycans is detected and measured using an immunoassay, a chemiluminescent assay, a fluorescent assay, a colorimetric assay, or a mass spectrometry assay. In one embodiment, the binding between the plurality of immunoglobulins and the plurality of glycans is detected and measured using the combination of LC-MS/MS and immunoassay techniques as described in Example 4.

In some embodiments, in a method in accordance with any one of the first, second, or third aspects described above, or any one of the fourth, fifth, sixth or seventh aspects as described below, including any one or more of the embodiments of these methods as described herein, the method further includes comparing the level of the glycan-binding pattern present in the GLF to a control level, for example, wherein the control level is a pre-determined level of the glycan-binding pattern present in the GLF of healthy subjects.

In some embodiments, in a method in accordance with the third aspect described above, or the sixth aspect described below, the method further comprises comparing the level of the glycan-binding pattern present in the GLF to a reference level; wherein the reference level is a level of the glycan-binding pattern present in the GLF that is detected and measured at an earlier pre-determined period of time, for example, at an earlier time when the subject is first diagnosed with pancreatic cancer, a GI tract cancer or predisposition to the same, or at an earlier time during a treatment regimen, etc., for example 2 weeks, 4 weeks, 8 weeks, 3 months, 6 months, 12 months, 18 months, or 24 months ago; or 3, 5, or 10 years ago.

### Gastrointestinal (GI) Tract Cancers

### Diagnostic Methods of a Gastrointestinal (GI) Tract Cancer

In a fourth aspect, the present disclosure relates to a method for diagnosing a gastrointestinal (GI) tract cancer or predisposition thereto (i.e., assessing whether a subject is afflicted with a GI tract cancer or predisposition thereto), by: (i) obtaining a sample including gastrointestinal lavage fluid (GLF) from the subject, wherein the GLF includes immunoglobulin A (IgA); (ii) contacting the GLF with a plurality of glycans; and (iii) detecting binding between the IgA and the plurality of glycans, thereby detecting a glycan-binding pattern; wherein the glycan-binding pattern is associated with the gastrointestinal (GI) tract cancer or predisposition thereto.

In a fifth aspect, the present disclosure relates to a method for detecting a glycan-binding pattern associated with a gastrointestinal (GI) tract cancer or predisposition thereto in a subject, by: (i) obtaining a sample including gastrointestinal lavage fluid (GLF) from the subject, wherein the GLF includes immunoglobulin A (IgA); (ii) contacting the GLF with a plurality of glycans; and (iii) detecting binding between the IgA and the plurality of glycans, thereby detecting the glycan-binding pattern.

### Prognostic Methods of a Gastrointestinal (GI) Tract Cancer

In a sixth aspect, the present disclosure relates to a method for evaluating or monitoring the progression of a gastrointestinal (GI) tract cancer or the efficacy of a gastrointestinal (GI) tract cancer treatment via a glycan-binding pattern associated with the gastrointestinal (GI) tract cancer or predisposition thereto in a subject, by: (i) obtaining a sample including gastrointestinal lavage fluid (GLF) from the subject, wherein the GLF includes immunoglobulin A (IgA); (ii) contacting the GLF with a plurality of glycans; and (iii) detecting binding between the IgA and the plurality of glycans, thereby detecting the glycan-binding pattern.

In some embodiments, in a method in accordance with any one of the fourth, fifth or sixth aspects described above, the glycan-binding pattern includes at least one glycan associated with GI tract cancer, e.g., colon cancer or colon adenoma, or predisposition thereto. In one embodiment, the glycan-binding pattern includes at least two glycans associated with GI cancer, e.g., colon cancer or colon adenoma, or predisposition thereto. In one embodiment, the glycan-binding pattern includes a glycan motif found in at least one glycan associated with the GI tract cancer, e.g., colon cancer or colon adenoma, or a predisposition thereto. In one embodiment, the at least one glycan is pre-determined to be bound at the highest intensity by the IgA in GLF in GI tract cancer patients. Alternatively or additionally, the at least one glycan associated with GI tract cancer, e.g., colon cancer or colon adenoma, or predisposition thereto is pre-determined to be bound at the lowest intensity by the IgA in GLF in GI tract cancer patients.

In certain embodiments, in a method in accordance with any one of the fourth, fifth or sixth aspects, including any one or more of the embodiments of these methods as described herein, the GI tract cancer is any cancer of any GI tract organ as defined above. In one embodiment, in a method in accordance with any one of the fourth, fifth or sixth aspects, including any one or more of the embodiments of these methods as described above, the GI tract cancer is pancreatic cancer. In one embodiment, in a method in accordance with any one of the fourth, fifth or sixth aspects, including any one or more of the embodiments of these methods as described above, the GI tract cancer is pancreatic ductal adenocarcinoma (PDAC). In one embodiment, the at least one glycan pre-determined to be bound at the highest intensity by the IgA in GLF from pancreatic cancer or PDAC patients includes the glycan motif GlcNAcα1-4Galβ1-4GlcNAc. In one embodiment, the at least one glycan pre-determined to be bound at the highest intensity by the IgA in GLF from pancreatic cancer or PDAC patients is selected from the group consisting of:

### GlcNAcα1-4Galβ1-4GlcNAcβ1-3Galβ1-4GlcNAcβ1-3Galβ1-4GlcNAc;

### GlcNAcα1-4Galβ1-4GlcNAc;

### GlcNAcα1-4Galβ1-4GlcNAcβ1-3Galβ1-4Glc;

### GlcNAcα1-4Galβ1-4GlcNAcβ1-3Galβ1-4(Fucα1-3)GlcNAcβ1-3Galβ1-4(Fucα1-3)GlcNAc;

### GlcNAcα1-4Galβ1-4GlcNAcβ1-3Galβ1-4GlcNAc; and

### GlcNAcβ1-3Galβ1-4GlcNAcβ1-3Galβ1-4GlcNAcβ1-2Manα1-6(GlcNAcβ1-3Galβ1-4GlcNAcβ1-3Galβ1-4GlcNAcβ1-2Manα1-3)Manβ1-4GlcNAcβ1-4GlcNAc.

In one embodiment, the at least one glycan pre-determined to be bound at the lowest intensity by the IgA in GLF from pancreatic cancer or PDAC patients is selected from the group consisting of:

### GlcNAcβ1-6Galβ1-4GlcNAc;

### GlcA;

### Galβ1-4(Fucα1-3)(6 S)Glc; and

### Manα1-6(Manα1-3)Manα1-6(Manα1-3)Man.

In some embodiments, in a method in accordance with any one of the fourth, fifth or sixth aspects, including any one or more of the embodiments of these methods as described herein, the GI tract cancer is colon cancer. In one embodiment, the at least one glycan pre-determined to be bound at the highest intensity by the IgA in GLF from colon cancer patients comprises one or more glycan motifs selected from the group consisting of:

### Galα1-3Galβ1-4GlcNAc;

### Galα1-3 Galβ1-4(Fuc); and

### Galα1-3Galβ1-4(Fucα1-3)GlcNAc.

In one embodiment, the at least one glycan pre-determined to be bound at the highest intensity by the IgA in GLF from colon cancer patients is selected from the group consisting of:

### Galα1-3Galβ1-4(Fucα1-3)GlcNAc;

### Galα1-3Galβ1-4GlcNAcβ1-2Manα1-6(Galα1-3Galβ1-4GlcNAcβ1-2Manα1-3)Manβ1-4GlcNAcβ1-4GlcNAc;

### Galα1-3Galβ1-4(Fucα1-3)GlcNAcβ1-2(Galα1-3Galβ1-4(Fucα1-3)GlcNAcβ1-2GalNAc;

### Galα1-3Galβ1-4GlcNAcβ1-2Manα1-6(Galα1-3Galβ1-4GlcNAcβ1-2Manα1-3)Manβ1-4GlcNAcβ1-4GlcNAc;

### Galα1-3Galβ1-4(Fucα1-3)GalNAc;

### Galα1-3Galβ1-4(Fucα1-3)GlcNAcβ1-2Manα1-6(Galα1-3Galβ1-4(Fucα1-3)GlcNAc; and

### Galα1-3Galβ1-4(Fucα1-3)GlcNAc.

In certain embodiments, in a method in accordance with any one of the fourth, fifth or sixth aspects, including any one or more of the embodiments of these methods as described above, the GI tract cancer is colon adenoma. In one embodiment, the at least one glycan pre-determined to be bound at the highest intensity by the IgA in GLF from colon adenoma patients comprises one or more glycan motifs selected from the group consisting of:

### Galβ1-4GlcNAcβ1-3Galβ1-4GlcNAc; and

### Galβ1-4GlcNAc.

In one embodiment, the at least one glycan pre-determined to be bound at the highest intensity by the IgA in GLF from colon adenoma patients is selected from the group consisting of:

### Fucα1-2Galβ1-4(Fucα1-3)GlcNAcβ1-2Manα1-6(Fucα1-2Galβ1-4(Fucα1-3)GlcNAcβ1-2Manα1-3)Manβ1-4GlcNAcβ1-4GlcNAc;

### Galβ1-4GlcNAcβ1-6(Galβ1-4GlcNAcβ1-2)Manα1-6(Galβ1-4GlcNAcβ1-2Manα1-3)Manβ1-4GlcNAcβ1-4GlcNAc;

### Galβ1-4GlcNAcβ1-3Galβ1-4GlcNAcβ1-3 Galβ1-4GlcNAcβ1-2Manα1-6(Galβ1-4GlcNAcβ1-3 Galβ1-4GlcNAcβ1-3Galβ1-4GlcNAcβ1-2Manα1-3)Manβ1-4GlcNAcβ1-4GlcNAc;

Galβ1-4GlcNAcβ1-3Galβ1-4GlcNAcβ1-3Galβ1-4GlcNAcβ1-3Galβ1-4GlcNAcβ1-2Manα1-6(Galβ1-4GlcNAcβ1-3Galβ1-4GlcNAcβ1-3Galβ1-4GlcNAcβ1-3Galβ1-4GlcNAcβ1-2Manα1-3)Manβ1-4GlcNAcβ1-4GlcNAc; and

### Galβ1-4(Fucα1-3)GlcNAc.

### Methods of Identifying Glycans

In a seventh aspect, the present disclosure relates to a method for identifying a glycan associated with a gastrointestinal (GI) tract cancer, by: (i) obtaining a sample including gastrointestinal lavage fluid (GLF) from a subject, wherein the GLF includes immunoglobulin A (IgA); (ii) contacting the GLF with a plurality of glycans; and (iii) detecting binding between the IgA and the plurality of glycans, thereby identifying the glycan. The method may further involve obtaining a sample comprising gastrointestinal lavage fluid (GLF) from at least one control subject, wherein the GLF comprises immunoglobulin A (IgA); contacting the GLF from the at least one control subject with a plurality of glycans; detecting binding between the IgA and the plurality of glycans; and identifying at least one glycan which binds at significantly different levels in GLF from the at least one subject with GI tract cancer as compared to the GLF from the at least one control subject.

As used herein, “a difference” or “at significantly different levels” refers broadly to any clinically relevant difference (including an increase or a decrease) and/or statistically significant difference in the level of IgA-glycan binding between the test sample and the control sample. In an exemplary embodiment, the difference is selected based on a cutoff value determined using a receiver operating characteristic (ROC) analysis, an example of which is presented in the examples. The optimal cutoff value may vary depending on the assay methods and conditions employed. In other embodiments, the difference must be greater than the limits of detection of the method for determining the level of glycan IgA binding. It is preferred that the difference be at least greater than the standard error of the assessment method, and preferably a difference of at least about 2-, about 3-, about 4-, about 5-, about 6-, about 7-, about 8-, about 9-, about 10-, about 15-, about 20-, about 25-, about 100-, about 500-, about 1000-fold or greater than the standard error of the assessment method. The difference may be assessed by any appropriate comparison, including any appropriate parametric or nonparametric descriptive statistic or comparison. For example, “an increase” in the level of binding may refer to a level that exceeds a cutoff value determined using an ROC analysis. It may also refer to the binding level of the IgA from the GLF from a subject with a GI tract cancer that is two, and more preferably about 5%, about 10%, about 15%, about 20%, about 25%, about 30%, about 40%, about 50%, about 60%, about 70%, about 80%, about 90%, about 100%, about 150%, about 200%, about 300%, about 400%, about 500%, about 600%, about 700%, about 800%, about 900% or about 1000% more than the binding level of the IgA from the GLF from the control subject. An increase may also refer to a level in a test sample that is preferably at least about 1.5, and more preferably about two, about three, about four, about five or more standard deviations above the average level of binding of the IgA from the GLF from the control subject. Likewise, “a decrease” in the level of binding of IgA from the GLF from a subject with a GI tract cancer may refer to a level that does not exceed a cutoff value determined using an ROC analysis. It may also refer to a level that is about 5%, about 10%, about 15%, about 20%, about 25%, about 30%, about 40%, about 50%, about 60%, about 70%, about 80%, or about 90% less than the binding level in the sample from the control subject. A decrease may also refer to a level in a test sample that is preferably at least about 1.5, and more preferably about two, about three, about four, about five or more standard deviations below the average level of binding of the IgA from the GLF from the control subject.

In a particular embodiment of the seventh aspect, the method involves obtaining samples comprising GLF from a plurality of subjects suffering from a GI tract cancer and/or obtaining samples comprising GLF from a plurality of control subjects.

In some embodiments of the method in accordance with the seventh aspect, the GI tract cancer is pancreatic cancer. In one embodiment, the GI tract cancer is pancreatic ductal adenocarcinoma (PDAC). In one embodiment, the glycan comprises a glycan motif. In one embodiment, the glycan motif is GlcNAcα1-4Galβ1-4GlcNAc.

In some embodiments of the method in accordance with the seventh aspect, the GI tract cancer is colon cancer. In one embodiment, the glycan comprises a glycan motif. In one embodiment, the glycan motif is one or more of the following:

### Galα1-3Galβ1-4GlcNAc;

### Galα1-3Galβ1-4(Fuc); and

### Galα1-3Galβ1-4(Fucα1-3)GlcNAc.

In some embodiments of the method in accordance with the seventh aspect, the GI tract cancer is colon adenoma. In one embodiment, the glycan comprises a glycan motif.

In one embodiment, the glycan motif is one or more of the following:

### Galβ1-4GlcNAcβ1-3Galβ1-4GlcNAc; and

### Galβ1-4GlcNAc.

### Lavage Compositions

Generally, lavage compositions for oral administration include solutions of electrolytes, such as sodium, potassium and magnesium salts of sulfate, bicarbonate, chloride, phosphate or citrate. Some such compositions may also include polyethylene glycol, which can act as a non-absorbable osmotic agent. Generic compositions include polyethylene glycol with an electrolyte solution, optionally also including bisacodyl, or ascorbic acid, and compositions including sulfate salts such as sodium sulfate, magnesium sulfate, or potassium sulfate. In some embodiments, an oral lavage fluid can include magnesium citrate. In some embodiments, an oral lavage fluid can include sodium picosulfate. One example composition of an oral lavage solution comprising polyethylene glycol with an electrolyte solution is GOLYTELY (Braintree Labs. Inc.). GOLYTELY is formulated according to the following: polyethylene glycol 59 g, sodium sulfate 5.68 g, sodium bicarbonate 1.69 g, sodium chloride 1.46 g, potassium chloride 0.745 g and water to make up one liter. Ingestion of GOLYTELY produces a voluminous, liquid stool with minimal changes in the subject's water and electrolyte balance. Another example of an oral lavage composition comprising polyethylene glycol with an electrolyte solution is NULYTELY (Braintree Labs. Inc.). An exemplary oral lavage composition comprising polyethylene glycol with an electrolyte solution and bisacodyl is HALFLYTELY (Braintree Labs. Inc.). An exemplary oral lavage composition comprising sulfate salts, such as sodium sulfate, magnesium sulfate, or potassium sulfate is SUPREP (Braintree Labs. Inc.). An exemplary composition of an oral lavage solution comprising polyethylene glycol with an electrolyte solution and ascorbic acid is MOVIPREP (Salix Pharmaceuticals, Inc.).

Polyethylene glycol is effective as an oral lavage composition when large amounts of polyethylene glycol are administered in large volumes of a dilute salt solution. Usually about 250-400 g polyethylene glycol is administered to the subject in about 4 L of an electrolyte solution in water. Oral administration of polyethylene glycol can be used to produce a bowel movement over a period of time, e.g., overnight. The dose required will vary, but from about 10-100 g of polyethylene glycol in 8 oz. of water can be effective. A dose of from about 68-85 g of polyethylene glycol can be effective to produce an overnight bowel movement, without profuse diarrhea. A volume of a solution of polyethylene glycol in an isotonic fluid can be an effective amount of an osmotic laxative. Volumes from about 0.5 L to about 4 L can be effective. Preferably, the effective volume is between about 1.5 L and about 2.5 L. Oral administration of 2 L of isotonic solution is effective.

More examples of oral lavage compositions include hypertonic solutions of non-phosphate salts with an osmotic laxative agent such as polyethylene glycol (U.S. Pat. App. No. 20090258090, incorporated by reference in its entirety). Mixtures of sulfate salts that omit phosphates, for example, effective amounts of one or more of the following sulfate salts Na₂SO₄, MgSO₄, and K₂SO₄ can be effective (e.g., SUPREP). Some embodiments include about 0.1 g to about 20.0 g Na₂SO₄, and from about 1.0 g to 10.0 g Na₂SO₄ may be useful. Dosage amounts of MgSO₄ from about 0.01 g to about 40.0 g can be effective. Doses of from about 0.1 g to about 20.0 g Na₂SO₄ may also be advantageously used, as well as dosages of 1.0 to 10.0 g. Dosage amounts of K₂SO₄ from about 0.01 g to about 20.0 g can be effective to produce purgation, and doses of from about 0.1 g to about 10.0 g and from about 0.5 g to about 5.0 g K₂SO₄ may also be useful. Addition of an osmotic laxative agent, such as polyethylene glycol (PEG) may improve the effectiveness of the above salt mixtures. Doses of PEG from about 1.0 g to about 100 g PEG are effective. Doses from about 10.0 g to about 50 g of PEG are also effective, as is a dose of about 34 g. For ease of administration, the above mixture of salts can be dissolved in a convenient volume of water. A volume of less than one liter of water can be well tolerated by most subjects. The mixture can be dissolved in any small volume of water, and volumes of between 100 and 500 ml are useful. The effective dose may be divided and administered to the patient in two or more administrations over an appropriate time period. Generally, administration of two doses of equal portions of the effective dose, separated by 6 to 24 hours produces satisfactory purgation. Some embodiments include cessation of normal oral intake during a defined period before and during administration of an oral lavage composition.

Some lavage compositions include a laxative, such as bisacodyl. In some embodiments, a laxative can be co-administered to a subject with a lavage composition. As will be understood, such co-administration can include, for example, administration of a laxative up to several hours before administration of a lavage composition to a subject, administration of a laxative with the administration of a lavage composition to a subject, or administration of a laxative up to several hours after administration of a lavage composition to a subject. Examples of laxatives and their effective doses include Aloe, 250-1000 mg.; Bisacodyl, about 5-80 mg.; Casanthranol, 30-360 mg.; Cascara aromatic fluid extract, 2-24 ml.; Cascara sagrada bark, 300-4000 mg.; Cascada sagrada extract, 300-2000 mg.; Cascara sagrada fluid extract, 0.5-5.0 ml.; Castor oil, 15-240 ml.; Danthron, 75-300 mg.; Dehydrocholic Acid, 250-2000 mg; Phenolphthalein, 30-1000 mg.; Sennosides A and B, 12-200 mg.; and Picosulfate, 1-100 mg.

More examples of lavage compositions include aqueous solutions of concentrated phosphate salts. The aqueous phosphate salt concentrate produces an osmotic effect on the intra-luminal contents of the GI tract; evacuation of the bowel occurs with a large influx of water and electrolytes into the colon from the body. One exemplary composition comprises 480 g/L monobasic sodium phosphate and 180 g/L dibasic sodium phosphate in stabilized buffered aqueous solution (FLEET'S PHOSPHO-SODA, C. S. Fleet Co., Inc.). Subjects are typically required to take 2-3 oz doses of this composition, separated by a three to 12 hour interval for a total of 6 ounces (180 ml).

### Techniques for Detection and Quantification of Glycan-Binding by GLF Immunoglobulins and Determination of Glycan-Binding Patterns

Examples of immunoassays include but are not limited to radioimmunoassays, enzyme-linked immunoassays (ELISA) and two-antibody sandwich assays as described herein. A variety of immunoassay formats, including competitive and non-competitive immunoassay formats, antigen capture assays and two-antibody sandwich assays also are useful (Self and Cook, (1996) Curr. Opin. Biotechnol. 7:60-65; incorporated by reference in its entirety). Some embodiments include one or more glycan capture assays. In a glycan capture assay, the GLF immunoglobulin (e.g., IgA) is bound to a solid phase, and the glycans are added individually or as a mixture to the immobilized immunoglobulins. After unbound proteins are removed by washing, the amount of bound glycan can be quantitated, if desired, using, for example, a radioassay (Harlow and Lane, (1988) Antibodies A Laboratory Manual Cold Spring Harbor Laboratory: New York, incorporated by reference in its entirety). Immunoassays can be performed under conditions of antibody excess, or as antigen competitions, to quantitate the level of glycan binding by the GLF immunoglobulin.

In certain embodiments, the binding of glycans by the GLF immunoglobulins (e.g., IgA) can be detected and/or measured using chemiluminescent detection. For example, in certain embodiments, specific anti-antibodies to IgA present in the biological sample, e.g., GLF sample and an antibody specific for IgA contained in the GLF and labeled with a chemiluminescent label is used to detect the glycan-binding by the immunoglobulins present in the GLF. Any chemiluminescent label and detection system can be used in the present methods. Chemiluminescent secondary antibodies can be obtained commercially from various sources such as Amersham. Methods of detecting chemiluminescent secondary antibodies are known in the art.

Fluorescent detection also can be useful for detecting the glycan-binding by the GLF immunoglobulins (e.g., IgA) in certain methods provided herein. Useful fluorochromes include, DAPI, fluorescein, Hoechst 33258, R-phycocyanin, B-phycoerythrin, R-phycoerythrin, rhodamine, Texas red and lissamine. Fluorescein or rhodamine labeled antibodies, or fluorescein- or rhodamine-labeled secondary antibodies can be useful in a method of the invention, for example, to bind to GLF immunoglobulins (e.g., IgA) after the immunoglobulins have been contacted or exposed to the plurality of glycans.

Radioimmunoassays (RIAs) also can be useful in certain methods provided herein. Such assays are well known in the art. Radioimmunoassays can be performed, for example, with ¹²⁵I-labeled primary or secondary antibody (Harlow and Lane, supra, 1988).

A signal from a detectable reagent can be analyzed, for example, using a spectrophotometer to detect color from a chromogenic substrate; a radiation counter to detect radiation, such as a gamma counter for detection of ¹²⁵I; or a fluorometer to detect fluorescence in the presence of light of a certain wavelength. Where an enzyme-linked assay is used, quantitative analysis of the amount of a target molecule can be performed using a spectrophotometer such as an EMAX Microplate Reader (Molecular Devices; Menlo Park, Calif.) in accordance with the manufacturer's instructions. The assays of the invention can be automated or performed robotically, if desired, and that the signal from multiple samples can be detected simultaneously.

In some embodiments, capillary electrophoresis based immunoassays (CEIA), which can be automated if desired, may be used to detect and/or measure the binding of glycans by the GLF immunoglobulins (e.g., IgA). Immunoassays also can be used in conjunction with laser-induced fluorescence as described, for example, in Schmalzing and Nashabeh, (Electrophoresis, 1997, 18:2184-93), and Bao (J. Chromatogr. B. Biomed. Sci., 1997, 699:463-80). Liposome immunoassays, such as flow-injection liposome immunoassays and liposome immunosensors, also can be used to detect and determine the level of the binding of glycans by the GLF immunoglobulins (e.g., IgA) according to certain methods provided in Rongen et al. (J. Immunol. Methods, 1997, 204:105-133).

A particularly useful method for analysis binding of glycans by GLF immunoglobulins or a fragment thereof is mass spectrometry (U.S. Patent Application No. 2010/0279382). A number of mass spectrometry-based quantitative proteomics methods have been developed that identify the proteins contained in each sample and determine the relative abundance of each identified protein across samples (Flory et al., Trends Biotechnol., 2002, 20:S23-29; Aebersold, J. Am. Soc. Mass Spectrom., 2003, 14:685-695; Aebersold, J. Infect. Dis., 2003, 187 Suppl 2:S315-320 (2003); Patterson and Aebersold, Nat. Genet., 2003, 33 Suppl, 311-323; Aebersold and Mann, Nature, 2003 422:198-207; Aebersold, R. and Cravat, Trends Biotechnol., 2002, 20:S1-2; Aebersold and Goodlett, Chem. Rev., 2001, 101:269-295 (2001); Tao and Aebersold, Curr. Opin. Biotechnol. 14:110-118 (2003)). Generally, the GLF immunoglobulins are labeled to acquire an isotopic signature that identifies their sample of origin and provides the basis for accurate mass spectrometric quantification. Samples with different isotopic signatures are then combined and analyzed, typically by multidimensional chromatography tandem mass spectrometry. The resulting collision induced dissociation (CID) spectra are then assigned to peptide sequences and the relative abundance of each detected protein in each sample is calculated based on the relative signal intensities for the differentially isotopically labeled peptides of identical sequence.

Some embodiments can include enriching proteins and/or protein fractions of a GLF. Example methods can include protein precipitation, chromatography, such as reverse phase chromatography, size exclusion chromatography, ion exchange chromatography, liquid chromatography, as well as affinity capture, differential extraction methods and centrifugation. Proteins and/or protein fractions can be further examined using intact protein methods such as top-down proteomics or gel chromatography such as SDS-PAGE.

### Kits

A kit, system or composition for carrying out any one of the methods in accordance with any one of the first, second, third, fourth, fifth, sixth or seventh aspects, including any one or more of the embodiments of these methods as described above, is also contemplated in the present disclosure. The kit comprises at least a lavage composition and any embodiments thereof as defined herein and an agent for recognizing and binding the GLF IgA after the immunoglobulin has bound a glycan from the screening. In other words, a kit of the present disclosure comprises an agent for recognizing a GLF IgA-glycan complex, wherein the glycan is not an endogenous antigen. Other components of the kit can include a vessel for collecting a GLF sample from a subject. A vessel for collecting a GLF can include an insert for a toilet which captures the GLF or fecal sample and the like. In some embodiments, the vessel can include a material to stabilize and/or preserve GLF proteins and immunoglobulins, such as one or more enzyme inhibitors, denaturatants and/or isolated protease inhibitors. In a further embodiment, the kit includes instructions for collecting the sample and/or performing the assay.

### Other Diseases

Any method and composition described herein can be applied to the diagnosis and prognosis of other non-cancer diseases of the gastrointestinal tract. Non-limiting examples of diseases include non-cancer diseases of the gastrointestinal tract pancreatitis, pancreatic adenocarcinoma, gastrointestinal neuroendocrine tumors, gastric adenocarcinoma, colon adenocarcinoma, hepatocellular carcinoma, cholangiocarcinoma, gallbladder adenoccarcinoma, ulcerative colitis, and Crohn's disease. Some diseases relate to an inflammatory bowel disease (IBD). As used herein, the term “inflammatory bowel disease” can refer to a broad class of diseases characterized by inflammation of at least part of the gastrointestinal tract. IBD symptoms may include inflammation of the intestine and resulting in abdominal cramping and persistent diarrhea. Inflammatory bowel diseases include ulcerative colitis (UC), Crohn's disease (CD), indeterminate colitis, chronic colitis, discontinuous or patchy disease, ileal inflammation, extracolonic inflammation, granulomatous inflammation in response to ruptured crypts, aphthous ulcers, transmural inflammation, microscopic colitis, diverticulitis and diversion colitis. More examples of diseases include celiac sprue, malabsorption disorders, and other conditions of digestive tract, liver, pancreas, and biliary tree.

### Examples

The following examples describe the materials and methods used to carry out exemplary immunoglobulin glycan binding studies, in particular of IgA found in GLF samples collected from healthy subjects and patients having pancreatic and colon cancer (Examples 1-3); and the results from these studies (Examples 4-8).

### Example 1 GLF Collection Methods

Gastrointestinal lavage fluid samples were collected from 3 healthy persons, 2 colon cancer patients, and 1 patient with benign colon adenoma as a waste product during routine colonoscopy; and from 14 patients with confirmed diagnosis of PDAC ranging in stages from IA to IV. PDAC samples were collected with prior Institutional Review Board (IRB) approval. Non-PDAC samples did not require IRB approval as the fluid is a waste product (IRB exempt) that is discarded prior to all colonoscopies. All samples were collected using the bowel preparative solution SuPrep (Braintree Laboratories, Inc.) to eliminate bias in preparation solution. Crude GLF samples were collected using a trap in the waste line and promptly processed for long-term storage. Prior to storage, samples were centrifuged at 120×G at 4° C. for 25 min to remove any large particulates. The resulting supernatant was then spun by centrifugation at 11,000×G, further clarifying the samples and removing any remaining fine debris. Rocker et al. (Clin. Transl. Gastroenterol., 2016, 7, e174). Aliquots (1.8 ml) of GLF were then stored at 80° C. Blinded samples of undiluted GLF described above were provided to the Consortium of Functional Glycomics (CFG, www.functionalglycomics.org) under dry ice for glycan array screening.

### Example 2 LC-MS/MS Analysis of GLF

Liquid chromatography-tandem mass spectrometry (LC-MS/MS) was performed on a ThermoScientific LTQ-OrbiTrap XL instrument. All samples were run in triplicate technical replicates. The peptides were separated using a reverse phase C18 column and an Agilent 1200 Series LC system over a two-hour gradient. The instrument method was set to data-dependent acquisition including a full MS scan in the OrbiTrap and fragmentation on the top 5 peptides. Product ions were scanned in the linear ion trap. Increased coverage was obtained by using a dynamic exclusion window of 45 s.

Raw data were converted to Mascot generic files using Mascot Distiller and searched using Mascot against a nonredundant custom human NCBI reference database (RefNCBInr) which included antibody components. Search parameters included semi-tryptic digestion allowing for two missed cleavages, 10 ppm (ppm) peptide tolerance, and 0.6 Da (Da) MS/MS tolerance. Spectral counting was used across the top 20 proteins identified for IgA composition determination.

### Example 3 Glycan Array Analysis

Glycan microarrays used in this study were from the CFG. All arrays used were version 5.2 which contain 609 unique glycan structures in replicates of 6 that were covalently linked to N-hydroxysuccinimide derivatized glass slides as was detailed previously in Blixt et al. (2004, Proc. Natl. Acad. Sci. USA., 101:17033-17038). The glycans present on the array represent a varied range of both naturally-occurring and synthetically-formulated structures. The structures of the 609 glycans in the CFG version 5.2 microarray used in the present study are provided in Table 1 below using the modified IUPAC condensed linear text nomenclature. Table 2 provides the structures of the slide attachment linker attached to the glycans.

To allow for glycan binding, crude GLF with 1% BSA was incubated on the microarray for a standard amount of time followed by incubation with a FITC fluorescein-labeled secondary antibody (Goat anti-Human IgA ThermoFisher Scientific Cat#: 31577). IgA binding was determined by scanning the FITC signal at each glycan on the slide. The highest and lowest values from each individual structure were removed, and the remaining 4 values were averaged and reported in relative fluorescent units (RFU) per sample per structure.

TABLE 1
Structure of glycans in CFG version 5.2 microarray

| Glycan | |
|---|---|
| ID NO: | Structure |
| 1 | Gala-Sp8 |
| 2 | Glca-Sp8 |
| 3 | Mana-Sp8 |
| 4 | GalNAca-Sp8 |
| 5 | GalNAca-Sp15 |
| 6 | Fuca-Sp8 |
| 7 | Fuca-Sp9 |
| 8 | Rhaa-Sp8 |
| 9 | Neu5Aca-Sp8 |
| 10 | Neu5Aca-Sp11 |
| 11 | Neu5Acb-Sp8 |
| 12 | Galb-Sp8 |
| 13 | Glcb-Sp8 |
| 14 | Manb-Sp8 |
| 15 | GalNAcb-Sp8 |
| 16 | GlcNAcb-Sp0 |
| 17 | GlcNAcb-Sp8 |
| 18 | GlcN(Gc)b-Sp8 |
| 19 | Galb1-4GlcNAcb1-6(Galb1-4GlcNAcb1-3)GalNAca-Sp8 |
| 20 | Galb1-4GlcNAcb1-6(Galb1-4GlcNAcb1-3)GalNAc-Sp14 |
| 21 | GlcNAcb1-6(GlcNAcb1-4)(GlcNAcb1-3)GlcNAc-Sp8 |
| 22 | 6S(3S)Galb1-4(6S)GlcNAcb-Sp0 |
| 23 | 6S(3S)Galb1-4GlcNAcb-Sp0 |
| 24 | (3S)Galb1-4(Fuca1-3)(6S)Glc-Sp0 |
| 25 | (3S)Galb1-4Glcb-Sp8 |
| 26 | (3S)Galb1-4(6S)Glcb-Sp0 |
| 27 | (3S)Galb1-4(6S)Glcb-Sp8 |
| 28 | (3S)Galb1-3(Fuca1-4)GlcNAcb-Sp8 |
| 29 | (3S)Galb1-3GalNAca-Sp8 |
| 30 | (3S)Galb1-3GlcNAcb-Sp0 |
| 31 | (3S)Galb1-3GlcNAcb-Sp8 |
| 32 | (3S)Galb1-4(Fuca1-3)GlcNAc-Sp0 |
| 33 | (3S)Galb1-4(Fuca1-3)GlcNAc-Sp8 |
| 34 | (3S)Galb1-4(6S)GlcNAcb-Sp0 |
| 35 | (3S)Galb1-4(6S)GlcNAcb-Sp8 |
| 36 | (3S)Galb1-4GlcNAcb-Sp0 |
| 37 | (3S)Galb1-4GlcNAcb-Sp8 |
| 38 | (3S)Galb-Sp8 |
| 39 | (6S)(4S)Galb1-4GlcNAcb-Sp0 |
| 40 | (4S)Galb1-4GlcNAcb-Sp8 |
| 41 | (6P)Mana-Sp8 |
| 42 | (6S)Galb1-4Glcb-Sp0 |
| 43 | (6S)Galb1-4Glcb-Sp8 |
| 44 | (6S)Galb1-4GlcNAcb-Sp8 |
| 45 | (6S)Galb1-4(6S)Glcb-Sp8 |
| 46 | Neu5Aca2-3(6S)Galb1-4GlcNAcb-Sp8 |
| 47 | (6S)GlcNAcb-Sp8 |
| 48 | Neu5,9Ac₂a-Sp8 |
| 49 | Neu5,9Ac₂a2-6Galb1-4GlcNAcb-Sp8 |
| 50 | Mana1-6(Mana1-3)Manb1-4GlcNAcb1-4GlcNAcb-Sp12 |
| 51 | Mana1-6(Mana1-3)Manb1-4GlcNAcb1-4GlcNAcb-Sp13 |
| 52 | GlcNAcb1-2Mana1-6(GlcNAcb1-2Mana1-3)Manb1-4GlcNAcb1-4GlcNAcb- |
| | Sp12 |
| 53 | GlcNAcb1-2Mana1-6(GlcNAcb1-2Mana1-3)Manb1-4GlcNAcb1-4GlcNAcb- |
| | Sp1 |
| 54 | Galb1-4GlcNAcb1-2Mana1-6(Galb1-4GlcNAcb1-2Mana1-3)Manb1- |
| | 4GlcNAcb1-4GlcNAcb-Sp12 |
| 55 | Neu5Aca2-6Galb1-4GlcNAcb1-2Mana1-6(Neu5Aca2-6Galb1-4GlcNAcb1- |
| | 2Mana1-3)Manb1-4GlcNAcb1-4GlcNAcb-Sp12 |
| 56 | Neu5Aca2-6Galb1-4GlcNAcb1-2Mana1-6(Neu5Aca2-6Galb1-4GlcNAcb1- |
| | 2Man-a1-3)Manb1-4GlcNAcb1-4GlcNAcb-Sp21 |
| 57 | Neu5Aca2-6Galb1-4GlcNAcb1-2Mana1-6(Neu5Aca2-6Galb1-4GlcNAcb1- |
| | 2Mana1-3)Manb1-4GlcNAcb1-4GlcNAcb-Sp24 |
| 58 | Fuca1-2Galb1-3GalNAcb1-3Gala-Sp9 |
| 59 | Fuca1-2Galb1-3GalNAcb1-3Gala1-4Galb1-4Glcb-Sp9 |
| 60 | Fuca1-2Galb1-3(Fuca1-4)GlcNAcb-Sp8 |
| 61 | Fuca1-2Galb1-3GalNAca-Sp8 |
| 62 | Fuca1-2Galb1-3GalNAca-Sp14 |
| 63 | Fuca1-2Galb1-3GalNAcb1-4(Neu5Aca2-3)Galb1-4Glcb-Sp0 |
| 64 | Fuca1-2Galb1-3GalNAcb1-4(Neu5Aca2-3)Galb1-4Glcb-Sp9 |
| 65 | Fuca1-2Galb1-3GlcNAcb1-3Galb1-4Glcb-Sp8 |
| 66 | Fuca1-2Galb1-3GlcNAcb1-3Galb1-4Glcb-Sp10 |
| 67 | Fuca1-2Galb1-3GlcNAcb-Sp0 |
| 68 | Fuca1-2Galb1-3GlcNAcb-Sp8 |
| 69 | Fuca1-2Galb1-4(Fuca1-3)GlcNAcb1-3Galb1-4(Fuca1-3)GlcNAcb-Sp0 |
| 70 | Fuca1-2Galb1-4(Fuca1-3)GlcNAcb1-3Galb1-4(Fuca1-3)GlcNAcb1-3Galb1- |
| | 4(Fuca1-3)GlcNAcb-Sp0 |
| 71 | Fuca1-2Galb1-4(Fuca1-3)GlcNAcb-Sp0 |
| 72 | Fuca1-2Galb1-4(Fuca1-3)GlcNAcb-Sp8 |
| 73 | Fuca1-2Galb1-4GlcNAcb1-3Galb1-4GlcNAcb-Sp0 |
| 74 | Fuca1-2Galb1-4GlcNAcb1-3Galb1-4GlcNAcb1-3Galb1-4GlcNAcb-Sp0 |
| 75 | Fuca1-2Galb1-4GlcNAcb-Sp0 |
| 76 | Fuca1-2Galb1-4GlcNAcb-Sp8 |
| 77 | Fuca1-2Galb1-4Glcb-Sp0 |
| 78 | Fuca1-2Galb-Sp8 |
| 79 | Fuca1-3GlcNAcb-Sp8 |
| 80 | Fuca1-4GlcNAcb-Sp8 |
| 81 | Fucb1-3GlcNAcb-Sp8 |
| 82 | GalNAca1-3(Fuca1-2)Galb1-3GlcNAcb-Sp0 |
| 83 | GalNAca1-3(Fuca1-2)Galb1-4(Fuca1-3)GlcNAcb-Sp0 |
| 84 | (3S)Galb1-4(Fuca1-3)Glcb-Sp0 |
| 85 | GalNAca1-3(Fuca1-2)Galb1-4GlcNAcb-Sp0 |
| 86 | GalNAca1-3(Fuca1-2)Galb1-4GlcNAcb-Sp8 |
| 87 | GalNAca1-3(Fuca1-2)Galb1-4Glcb-Sp0 |
| 88 | GlcNAcb1-3Galb1-3GalNAca-Sp8 |
| 89 | GalNAca1-3(Fuca1-2)Galb-Sp8 |
| 90 | GalNAca1-3(Fuca1-2)Galb-Sp18 |
| 91 | GalNAca1-3GalNAcb-Sp8 |
| 92 | GalNAca1-3Galb-Sp8 |
| 93 | GalNAca1-4(Fuca1-2)Galb1-4GlcNAcb-Sp8 |
| 94 | GalNAcb1-3GalNAca-Sp8 |
| 95 | GalNAcb1-3(Fuca1-2)Galb-Sp8 |
| 96 | GalNAcb1-3Gala1-4Galb1-4GlcNAcb-Sp0 |
| 97 | GalNAcb1-4(Fuca1-3)GlcNAcb-Sp0 |
| 98 | GalNAcb1-4GlcNAcb-Sp0 |
| 99 | GalNAcb1-4GlcNAcb-Sp8 |
| 100 | Gala1-2Galb-Sp8 |
| 101 | Gala1-3(Fuca1-2)Galb1-3GlcNAcb-Sp0 |
| 102 | Gala1-3(Fuca1-2)Galb1-3GlcNAcb-Sp8 |
| 103 | Gala1-3(Fuca1-2)Galb1-4(Fuca1-3)GlcNAcb-Sp0 |
| 104 | Gala1-3(Fuca1-2)Galb1-4(Fuca1-3)GlcNAcb-Sp8 |
| 105 | Gala1-3(Fuca1-2)Galb1-4GlcNAc-Sp0 |
| 106 | Gala1-3(Fuca1-2)Galb1-4Glcb-Sp0 |
| 107 | Gala1-3(Fuca1-2)Galb-Sp8 |
| 108 | Gala1-3(Fuca1-2)Galb-Sp18 |
| 109 | Gala1-4(Gala1-3)Galb1-4GlcNAcb-Sp8 |
| 110 | Gala1-3GalNAca-Sp8 |
| 111 | Gala1-3GalNAca-Sp16 |
| 112 | Gala1-3GalNAcb-Sp8 |
| 113 | Gala1-3Galb1-4(Fuca1-3)GlcNAcb-Sp8 |
| 114 | Gala1-3Galb1-3GlcNAcb-Sp0 |
| 115 | Gala1-3Galb1-4GlcNAcb-Sp8 |
| 116 | Gala1-3Galb1-4Glcb-Sp0 |
| 117 | Gala1-3Galb1-4Glc-Sp10 |
| 118 | Gala1-3Galb-Sp8 |
| 119 | Gala1-4(Fuca1-2)Galb1-4GlcNAcb-Sp8 |
| 120 | Gala1-4Galb1-4GlcNAcb-Sp0 |
| 121 | Gala1-4Galb1-4GlcNAcb-Sp8 |
| 122 | Gala1-4Galb1-4Glcb-Sp0 |
| 123 | Gala1-4GlcNAcb-Sp8 |
| 124 | Gala1-6Glcb-Sp8 |
| 125 | Galb1-2Galb-Sp8 |
| 126 | Galb1-3(Fuca1-4)GlcNAcb1-3Galb1-4(Fuca1-3)GlcNAcb-Sp0 |
| 127 | Galb1-3GlcNAcb1-3Galb1-4(Fuca1-3)GlcNAcb-Sp0 |
| 128 | Galb1-3(Fuca1-4)GlcNAc-Sp0 |
| 129 | Galb1-3(Fuca1-4)GlcNAc-Sp8 |
| 130 | Fuca1-4(Galb1-3)GlcNAcb-Sp8 |
| 131 | Galb1-4GlcNAcb1-6GalNAca-Sp8 |
| 132 | Galb1-4GlcNAcb1-6GalNAc-Sp14 |
| 133 | GlcNAcb1-6(Galb1-3)GalNAca-Sp8 |
| 134 | GlcNAcb1-6(Galb1-3)GalNAca-Sp14 |
| 135 | Neu5Aca2-6(Galb1-3)GalNAca-Sp8 |
| 136 | Neu5Aca2-6(Galb1-3)GalNAca-Sp14 |
| 137 | Neu5Acb2-6(Galb1-3)GalNAca-Sp8 |
| 138 | Neu5Aca2-6(Galb1-3)GlcNAcb1-4Galb1-4Glcb-Sp10 |
| 139 | Galb1-3GalNAca-Sp8 |
| 140 | Galb1-3GalNAca-Sp14 |
| 141 | Galb1-3GalNAca-Sp16 |
| 142 | Galb1-3GalNAcb-Sp8 |
| 143 | Galb1-3GalNAcb1-3Gala1-4Galb1-4Glcb-Sp0 |
| 144 | Galb1-3GalNAcb1-4(Neu5Aca2-3)Galb1-4Glcb-Sp0 |
| 145 | Galb1-3GalNAcb1-4Galb1-4Glcb-Sp8 |
| 146 | Galb1-3Galb-Sp8 |
| 147 | Galb1-3GlcNAcb1-3Galb1-4GlcNAcb-Sp0 |
| 148 | Galb1-3GlcNAcb1-3Galb1-4Glcb-Sp10 |
| 149 | Galb1-3GlcNAcb-Sp0 |
| 150 | Galb1-3GlcNAcb-Sp8 |
| 151 | Galb1-4(Fuca1-3)GlcNAcb-Sp0 |
| 152 | Galb1-4(Fuca1-3)GlcNAcb-Sp8 |
| 153 | Galb1-4(Fuca1-3)GlcNAcb1-3Galb1-4(Fuca1-3)GlcNAcb-Sp0 |
| 154 | Galb1-4(Fuca1-3)GlcNAcb1-3Galb1-4(Fuca1-3)GlcNAcb1-3Galb1-4(Fuca1- |
| | 3)GlcNAcb-Sp0 |
| 155 | Galb1-4(6S)Glcb-Sp0 |
| 156 | Galb1-4(6S)Glcb-Sp8 |
| 157 | Galb1-4GalNAca1-3(Fuca1-2)Galb1-4GlcNAcb-Sp8 |
| 158 | Galb1-4GalNAcb1-3(Fuca1-2)Galb1-4GlcNAcb-Sp8 |
| 159 | Galb1-4GlcNAcb1-3GalNAca-Sp8 |
| 160 | Galb1-4GlcNAcb1-3GalNAc-Sp14 |
| 161 | Galb1-4GlcNAcb1-3Galb1-4(Fuca1-3)GlcNAcb1-3Galb1-4(Fuca1- |
| | 3)GlcNAcb-Sp0 |
| 162 | Galb1-4GlcNAcb1-3Galb1-4GlcNAcb1-3Galb1-4GlcNAcb-Sp0 |
| 163 | Galb1-4GlcNAcb1-3Galb1-4GlcNAcb-Sp0 |
| 164 | Galb1-4GlcNAcb1-3Galb1-4Glcb-Sp0 |
| 165 | Galb1-4GlcNAcb1-3Galb1-4Glcb-Sp8 |
| 166 | Galb1-4GlcNAcb1-6(Galb1-3)GalNAca-Sp8 |
| 167 | Galb1-4GlcNAcb1-6(Galb1-3)GalNAc-Sp14 |
| 168 | Galb1-4GlcNAcb-Sp0 |
| 169 | Galb1-4GlcNAcb-Sp8 |
| 170 | Galb1-4GlcNAcb-Sp23 |
| 171 | Galb1-4Glcb-Sp0 |
| 172 | Galb1-4Glcb-Sp8 |
| 173 | GlcNAca1-3Galb1-4GlcNAcb-Sp8 |
| 174 | GlcNAca1-6Galb1-4GlcNAcb-Sp8 |
| 175 | GlcNAcb1-2Galb1-3GalNAca-Sp8 |
| 176 | GlcNAcb1-6(GlcNAcb1-3)GalNAca-Sp8 |
| 177 | GlcNAcb1-6(GlcNAcb1-3)GalNAca-Sp14 |
| 178 | GlcNAcb1-6(GlcNAcb1-3)Galb1-4GlcNAcb-Sp8 |
| 179 | GlcNAcb1-3GalNAca-Sp8 |
| 180 | GlcNAcb1-3GalNAca-Sp14 |
| 181 | GlcNAcb1-3Galb-Sp8 |
| 182 | GlcNAcb1-3Galb1-4GlcNAcb-Sp0 |
| 183 | GlcNAcb1-3Galb1-4GlcNAcb-Sp8 |
| 184 | GlcNAcb1-3Galb1-4GlcNAcb1-3Galb1-4GlcNAcb-Sp0 |
| 185 | GlcNAcb1-3Galb1-4Glcb-Sp0 |
| 186 | GlcNAcb1-4-MDPLys |
| 187 | GlcNAcb1-6(GlcNAcb1-4)GalNAca-Sp8 |
| 188 | GlcNAcb1-4Galb1-4GlcNAcb-Sp8 |
| 189 | GlcNAcb1-4GlcNAcb1-4GlcNAcb1-4GlcNAcb1-4GlcNAcb1-4GlcNAcb1- |
| | Sp8 |
| 190 | GlcNAcb1-4GlcNAcb1-4GlcNAcb1-4GlcNAcb1-4GlcNAcb1-Sp8 |
| 191 | GlcNAcb1-4GlcNAcb1-4GlcNAcb-Sp8 |
| 192 | GlcNAcb1-6GalNAca-Sp8 |
| 193 | GlcNAcb1-6GalNAca-Sp14 |
| 194 | GlcNAcb1-6Galb1-4GlcNAcb-Sp8 |
| 195 | Glca1-4Glcb-Sp8 |
| 196 | Glca1-4Glca-Sp8 |
| 197 | Glca1-6Glca1-6Glcb-Sp8 |
| 198 | Glcb1-4Glcb-Sp8 |
| 163 | Galb1-4GlcNAcb1-3Galb1-4GlcNAcb-Sp0 |
| 199 | Glcb1-6Glcb-Sp8 |
| 200 | G-ol-Sp8 |
| 201 | GlcAa-Sp8 |
| 202 | GlcAb-Sp8 |
| 203 | GlcAb1-3Galb-Sp8 |
| 204 | GlcAb1-6Galb-Sp8 |
| 205 | KDNa2-3Galb1-3GlcNAcb-Sp0 |
| 206 | KDNa2-3Galb1-4GlcNAcb-Sp0 |
| 207 | Mana1-2Mana1-2Mana1-3Mana-Sp9 |
| 208 | Mana1-2Mana1-6(Mana1-2Mana1-3)Mana-Sp9 |
| 209 | Mana1-2Mana1-3Mana-Sp9 |
| 210 | Mana1-6(Mana1-2Mana1-3)Mana1-6(Mana1-2Mana1-3)Manb1-4GlcNAcb1- |
| | 4GlcNAcb-Sp12 |
| 211 | Mana1-2Mana1-6(Mana1-3)Mana1-6(Mana1-2Mana1-2Mana1-3)Manb1- |
| | 4GlcNAcb1-4GlcNAcb-Sp12 |
| 212 | Mana1-2Mana1-6(Mana1-2Mana1-3)Mana1-6(Mana1-2Mana1-2Mana1- |
| | 3)Manb1-4GlcNAcb1-4GlcNAcb-Sp12 |
| 213 | Mana1-6(Mana1-3)Mana-Sp9 |
| 214 | Mana1-2Mana1-2Mana1-6(Mana1-3)Mana-Sp9 |
| 215 | Mana1-6(Mana1-3)Mana1-6(Mana1-2Mana1-3)Manb1-4GlcNAcb1- |
| | 4GlcNAcb-Sp12 |
| 216 | Mana1-6(Mana1-3)Mana1-6(Mana1-3)Manb1-4GlcNAcb1-4GlcNAcb-Sp12 |
| 217 | Manb1-4GlcNAcb-Sp0 |
| 218 | Neu5Aca2-3Galb1-4GlcNAcb1-3Galb1-4(Fuca1-3)GlcNAcb-Sp0 |
| 219 | (3S)Galb1-4(Fuca1-3)(6S)GlcNAcb-Sp8 |
| 220 | Fuca1-2(6S)Galb1-4GlcNAcb-Sp0 |
| 221 | Fuca1-2Galb1-4(6S)GlcNAcb-Sp8 |
| 222 | Fuca1-2(6S)Galb1-4(6S)Glcb-Sp0 |
| 223 | Neu5Aca2-3Galb1-3GalNAca-Sp8 |
| 224 | Neu5Aca2-3Galb1-3GalNAca-Sp14 |
| 225 | GalNAcb1-4(Neu5Aca2-8Neu5Aca2-8Neu5Aca2-8Neu5Aca2-3)Galb1- |
| | 4Glcb-Sp0 |
| 226 | GalNAcb1-4(Neu5Aca2-8Neu5Aca2-8Neu5Aca2-3)Galb1-4Glcb-Sp0 |
| 227 | Neu5Aca2-8Neu5Aca2-8Neu5Aca2-3Galb1-4Glcb-Sp0 |
| 228 | GalNAcb1-4(Neu5Aca2-8Neu5Aca2-3)Galb1-4Glcb-Sp0 |
| 229 | Neu5Aca2-8Neu5Aca2-8Neu5Aca-Sp8 |
| 230 | Neu5Aca2-3(6S)Galb1-4(Fuca1-3)GlcNAcb-Sp8 |
| 231 | GalNAcb1-4(Neu5Aca2-3)Galb1-4GlcNAcb-Sp0 |
| 232 | GalNAcb1-4(Neu5Aca2-3)Galb1-4GlcNAcb-Sp8 |
| 233 | GalNAcb1-4(Neu5Aca2-3)Galb1-4Glcb-Sp0 |
| 234 | Neu5Aca2-3Galb1-3GalNAcb1-4(Neu5Aca2-3)Galb1-4Glcb-Sp0 |
| 235 | Neu5Aca2-6(Neu5Aca2-3)GalNAca-Sp8 |
| 236 | Neu5Aca2-3GalNAca-Sp8 |
| 237 | Neu5Aca2-3GalNAcb1-4GlcNAcb-Sp0 |
| 238 | Neu5Aca2-3Galb1-3(6S)GlcNAc-Sp8 |
| 239 | Neu5Aca2-3Galb1-3(Fuca1-4)GlcNAcb-Sp8 |
| 240 | Neu5Aca2-3Galb1-3(Fuca1-4)GlcNAcb1-3Galb1-4(Fuca1-3)GlcNAcb-Sp0 |
| 241 | Neu5Aca2-3Galb1-4(Neu5Aca2-3Galb1-3)GlcNAcb-Sp8 |
| 242 | Neu5Aca2-3Galb1-3(6S)GalNAca-Sp8 |
| 243 | Neu5Aca2-6(Neu5Aca2-3Galb1-3)GalNAca-Sp8 |
| 244 | Neu5Aca2-6(Neu5Aca2-3Galb1-3)GalNAca-Sp14 |
| 245 | Neu5Aca2-3Galb-Sp8 |
| 246 | Neu5Aca2-3Galb1-3GalNAcb1-3Gala1-4Galb1-4Glcb-Sp0 |
| 247 | Neu5Aca2-3Galb1-3GlcNAcb1-3Galb1-4GlcNAcb-Sp0 |
| 248 | Fuca1-2(6S)Galb1-4Glcb-Sp0 |
| 249 | Neu5Aca2-3Galb1-3GlcNAcb-Sp0 |
| 250 | Neu5Aca2-3Galb1-3GlcNAcb-Sp8 |
| 251 | Neu5Aca2-3Galb1-4(6S)GlcNAcb-Sp8 |
| 252 | Neu5Aca2-3Galb1-4(Fuca1-3)(6S)GlcNAcb-Sp8 |
| 253 | Neu5Aca2-3Galb1-4(Fuca1-3)GlcNAcb1-3Galb1-4(Fuca1-3)GlcNAcb1- |
| | 3Galb1-4(Fuca1-3)GlcNAcb-Sp0 |
| 254 | Neu5Aca2-3Galb1-4(Fuca1-3)GlcNAcb-Sp0 |
| 255 | Neu5Aca2-3Galb1-4(Fuca1-3)GlcNAcb-Sp8 |
| 256 | Neu5Aca2-3Galb1-4(Fuca1-3)GlcNAcb1-3Galb-Sp8 |
| 257 | Neu5Aca2-3Galb1-4(Fuca1-3)GlcNAcb1-3Galb1-4GlcNAcb-Sp8 |
| 258 | Neu5Aca2-3Galb1-4GlcNAcb1-3Galb1-4GlcNAcb1-3Galb1-4GlcNAcb-Sp0 |
| 259 | Neu5Aca2-3Galb1-4GlcNAcb-Sp0 |
| 260 | Neu5Aca2-3Galb1-4GlcNAcb-Sp8 |
| 261 | Neu5Aca2-3Galb1-4GlcNAcb1-3Galb1-4GlcNAcb-Sp0 |
| 262 | Fuca1-2Galb1-4(6S)Glcb-Sp0 |
| 263 | Neu5Aca2-3Galb1-4Glcb-Sp0 |
| 264 | Neu5Aca2-3Galb1-4Glcb-Sp8 |
| 265 | Neu5Aca2-6GalNAca-Sp8 |
| 266 | Neu5Aca2-6GalNAcb1-4GlcNAcb-Sp0 |
| 267 | Neu5Aca2-6Galb1-4(6S)GlcNAcb-Sp8 |
| 268 | Neu5Aca2-6Galb1-4GlcNAcb-Sp0 |
| 269 | Neu5Aca2-6Galb1-4GlcNAcb-Sp8 |
| 270 | Neu5Aca2-6Galb1-4GlcNAcb1-3Galb1-4(Fuca1-3)GlcNAcb1-3Galb1- |
| | 4(Fuca1-3)GlcNAcb-Sp0 |
| 271 | Neu5Aca2-6Galb1-4GlcNAcb1-3Galb1-4GlcNAcb-Sp0 |
| 272 | Neu5Aca2-6Galb1-4Glcb-Sp0 |
| 273 | Neu5Aca2-6Galb1-4Glcb-Sp8 |
| 274 | Neu5Aca2-6Galb-Sp8 |
| 275 | Neu5Aca2-8Neu5Aca-Sp8 |
| 276 | Neu5Aca2-8Neu5Aca2-3Galb1-4Glcb-Sp0 |
| 277 | Galb1-3(Fuca1-4)GlcNAcb1-3Galb1-3(Fuca1-4)GlcNAcb-Sp0 |
| 278 | Neu5Acb2-6GalNAca-Sp8 |
| 279 | Neu5Acb2-6Galb1-4GlcNAcb-Sp8 |
| 280 | Neu5Gca2-3Galb1-3(Fuca1-4)GlcNAcb-Sp0 |
| 281 | Neu5Gca2-3Galb1-3GlcNAcb-Sp0 |
| 282 | Neu5Gca2-3Galb1-4(Fuca1-3)GlcNAcb-Sp0 |
| 283 | Neu5Gca2-3Galb1-4GlcNAcb-Sp0 |
| 284 | Neu5Gca2-3Galb1-4Glcb-Sp0 |
| 285 | Neu5Gca2-6GalNAca-Sp0 |
| 286 | Neu5Gca2-6Galb1-4GlcNAcb-Sp0 |
| 287 | Neu5Gca-Sp8 |
| 288 | Neu5Aca2-3Galb1-4GlcNAcb1-6(Galb1-3)GalNAca-Sp14 |
| 289 | Galb1-3GlcNAcb1-3Galb1-3GlcNAcb-Sp0 |
| 290 | Galb1-4(Fuca1-3)(6S)GlcNAcb-Sp0 |
| 291 | Galb1-4(Fuca1-3)(6S)Glcb-Sp0 |
| 292 | Galb1-4(Fuca1-3)GlcNAcb1-3Galb1-3(Fuca1-4)GlcNAcb-Sp0 |
| 293 | Galb1-4GlcNAcb1-3Galb1-3GlcNAcb-Sp0 |
| 294 | Neu5Aca2-3Galb1-3GlcNAcb1-3Galb1-3GlcNAcb-Sp0 |
| 295 | Neu5Aca2-3Galb1-4GlcNAcb1-3Galb1-3GlcNAcb-Sp0 |
| 296 | 4S(3S)Galb1-4GlcNAcb-Sp0 |
| 297 | (6S)Galb1-4(6S)GlcNAcb-Sp0 |
| 298 | (6P)Glcb-Sp10 |
| 299 | Neu5Aca2-3Galb1-4(Fuca1-3)GlcNAcb1-6(Galb1-3)GalNAca-Sp14 |
| 300 | Galb1-3Galb1-4GlcNAcb-Sp8 |
| 301 | Neu5Aca2-6Galb1-4GlcNAcb1-2Mana1-6(Galb1-4GlcNAcb1-2Mana1- |
| | 3)Manb1-4GlcNAcb1-4GlcNAcb-Sp12 |
| 302 | Galb1-4GlcNAcb1-6(Galb1-4GlcNAcb1-3)Galb1-4GlcNAc-Sp0 |
| 303 | GlcNAcb1-6(Galb1-4GlcNAcb1-3)Galb1-4GlcNAc-Sp0 |
| 304 | Galb1-4GlcNAca1-6Galb1-4GlcNAcb-Sp0 |
| 305 | Galb1-4GlcNAcb1-6Galb1-4GlcNAcb-Sp0 |
| 306 | GalNAcb1-3Galb-Sp8 |
| 307 | GlcAb1-3GlcNAcb-Sp8 |
| 308 | Neu5Aca2-6Galb1-4GlcNAcb1-2Mana1-6(GlcNAcb1-2Mana1-3)Manb1- |
| | 4GlcNAcb1-4GlcNAcb-Sp12 |
| 309 | GlcNAcb1-3Man-Sp10 |
| 310 | GlcNAcb1-4GlcNAcb-Sp10 |
| 311 | GlcNAcb1-4GlcNAcb-Sp12 |
| 312 | MurNAcb1-4GlcNAcb-Sp10 |
| 313 | Mana1-6Manb-Sp10 |
| 314 | Mana1-6(Mana1-3)Mana1-6(Mana1-3)Manb-Sp10 |
| 315 | Mana1-2Mana1-6(Mana1-3)Mana1-6(Mana1-2Mana1-2Mana1-3)Mana-Sp9 |
| 316 | Mana1-2Mana1-6(Mana1-2Mana1-3)Mana1-6(Mana1-2Mana1-2Mana1- |
| | 3)Mana-Sp9 |
| 317 | Neu5Aca2-3Galb1-4GlcNAcb1-6(Neu5Aca2-3Galb1-3)GalNAca-Sp14 |
| 318 | Neu5Aca2-6Galb1-4GlcNAcb1-2Mana1-6(Neu5Aca2-3Galb1-4GlcNAcb1- |
| | 2Mana1-3)Manb1-4GlcNAcb1-4GlcNAcb-Sp12 |
| 319 | Galb1-4GlcNAcb1-2Mana1-6(Neu5Aca2-6Galb1-4GlcNAcb1-2Mana1- |
| | 3)Manb1-4GlcNAcb1-4GlcNAcb-Sp12 |
| 320 | Neu5Aca2-8Neu5Acb-Sp17 |
| 321 | Neu5Aca2-8Neu5Aca2-8Neu5Acb-Sp8 |
| 322 | Neu5Gcb2-6Galb1-4GlcNAc-Sp8 |
| 323 | Galb1-3GlcNAcb1-2Mana1-6(Galb1-3GlcNAcb1-2Mana1-3)Manb1- |
| | 4GlcNAcb1-4GlcNAcb-Sp19 |
| 324 | Neu5Aca2-3Galb1-4GlcNAcb1-2Mana1-6(Neu5Aca2-3Galb1-4GlcNAcb1- |
| | 2Mana1-3)Manb1-4GlcNAcb1-4GlcNAcb-Sp12 |
| 325 | Neu5Aca2-3Galb1-4GlcNAcb1-2Mana1-6(Neu5Aca2-6Galb1-4GlcNAcb1- |
| | 2Mana1-3)Manb1-4GlcNAcb1-4GlcNAcb-Sp12 |
| 326 | Galb1-4(Fuca1-3)GlcNAcb1-2Mana1-6(Galb1-4(Fuca1-3)GlcNAcb1- |
| | 2Mana1-3)Manb1-4GlcNAcb1-4GlcNAcb-Sp20 |
| 327 | Neu5,9Ac2a2-3Galb1-4GlcNAcb-Sp0 |
| 328 | Neu5,9Ac2a2-3Galb1-3GlcNAcb-Sp0 |
| 329 | Neu5Aca2-6Galb1-4GlcNAcb1-3Galb1-3GlcNAcb-Sp0 |
| 330 | Neu5Aca2-3Galb1-3(Fuca1-4)GlcNAcb1-3Galb1-3(Fuca1-4)GlcNAcb-Sp0 |
| 331 | Neu5Aca2-6Galb1-4GlcNAcb1-3Galb1-4GlcNAcb1-3Galb1-4GlcNAcb-Sp0 |
| 332 | Gala1-4Galb1-4GlcNAcb1-3Galb1-4Glcb-Sp0 |
| 333 | GalNAcb1-3Gala1-4Galb1-4GlcNAcb1-3Galb1-4Glcb-Sp0 |
| 334 | GalNAca1-3(Fuca1-2)Galb1-4GlcNAcb1-3Galb1-4GlcNAcb-Sp0 |
| 335 | GalNAca1-3(Fuca1-2)Galb1-4GlcNAcb1-3Galb1-4GlcNAcb1-3Galb1- |
| | 4GlcNAcb-Sp0 |
| 336 | Neu5Aca2-3Galb1-4(Fuca1-3)GlcNAcb1-6(Neu5Aca2-3Galb1-3)GalNAc- |
| | Sp14 |
| 337 | GlcNAca1-4Galb1-4GlcNAcb1-3Galb1-4GlcNAcb1-3Galb1-4GlcNAcb-Sp0 |
| 338 | GlcNAca1-4Galb1-4GlcNAcb-Sp0 |
| 339 | GlcNAca1-4Galb1-3GlcNAcb-Sp0 |
| 340 | GlcNAca1-4Galb1-4GlcNAcb1-3Galb1-4Glcb-Sp0 |
| 341 | GlcNAca1-4Galb1-4GlcNAcb1-3Galb1-4(Fuca1-3)GlcNAcb1-3Galb1- |
| | 4(Fuca1-3)GlcNAcb-Sp0 |
| 342 | GlcNAca1-4Galb1-4GlcNAcb1-3Galb1-4GlcNAcb-Sp0 |
| 343 | GlcNAca1-4Galb1-3GalNAc-Sp14 |
| 344 | Neu5Aca2-6Galb1-4GlcNAcb1-2Mana1-6(Mana1-3)Manb1-4GlcNAcb1- |
| | 4GlcNAc-Sp12 |
| 345 | Mana1-6(Neu5Aca2-6Galb1-4GlcNAcb1-2Mana1-3)Manb1-4GlcNAcb1- |
| | 4GlcNAc-Sp12 |
| 346 | Neu5Aca2-6Galb1-4GlcNAcb1-2Mana1-6Manb1-4GlcNAcb1-4GlcNAc-Sp12 |
| 347 | Neu5Aca2-6Galb1-4GlcNAcb1-2Mana1-3Manb1-4GlcNAcb1-4GlcNAc-Sp12 |
| 348 | Galb1-4GlcNAcb1-2Mana1-3Manb1-4GlcNAcb1-4GlcNAc-Sp12 |
| 349 | Galb1-4GlcNAcb1-2Mana1-6Manb1-4GlcNAcb1-4GlcNAc-Sp12 |
| 350 | Mana1-6(Galb1-4GlcNAcb1-2Mana1-3)Manb1-4GlcNAcb1-4GlcNAcb-Sp12 |
| 351 | GlcNAcb1-2Mana1-6(GlcNAcb1-2Mana1-3)Manb1-4GlcNAcb1-4(Fuca1- |
| | 6)GlcNAcb-Sp22 |
| 352 | Galb1-4GlcNAcb1-2Mana1-6(Galb1-4GlcNAcb1-2Mana1-3)Manb1- |
| | 4GlcNAcb1-4(Fuca1-6)GlcNAcb-Sp22 |
| 353 | Galb1-3GlcNAcb1-2Mana1-6(Galb1-3GlcNAcb1-2Mana1-3)Manb1- |
| | 4GlcNAcb1-4(Fuca1-6)GlcNAcb-Sp22 |
| 354 | (6S)GlcNAcb1-3Galb1-4GlcNAcb-Sp0 |
| 355 | KDNa2-3Galb1-4(Fuca1-3)GlcNAc-Sp0 |
| 356 | KDNa2-6Galb1-4GlcNAc-Sp0 |
| 357 | KDNa2-3Galb1-4Glc-Sp0 |
| 358 | KDNa2-3Galb1-3GalNAca-Sp14 |
| 359 | Fuca1-2Galb1-3GlcNAcb1-2Mana1-6(Fuca1-2Galb1-3GlcNAcb1-2Mana1- |
| | 3)Manb1-4GlcNAcb1-4GlcNAcb-Sp20 |
| 360 | Fuca1-2Galb1-4GlcNAcb1-2Mana1-6(Fuca1-2Galb1-4GlcNAcb1-2Mana1- |
| | 3)Manb1-4GlcNAcb1-4GlcNAcb-Sp20 |
| 361 | Fuca1-2Galb1-4(Fuca1-3)GlcNAcb1-2Mana1-6(Fuca1-2Galb1-4(Fuca1- |
| | 3)GlcNAcb1-2Mana1-3)Manb1-4GlcNAcb1-4GlcNAcb-Sp20 |
| 362 | Gala1-3Galb1-4GlcNAcb1-2Mana1-6(Gala1-3Galb1-4GlcNAcb1-2Mana1- |
| | 3)Manb1-4GlcNAcb1-4GlcNAcb-Sp20 |
| 363 | Galb1-4GlcNAcb1-2Mana1-6(Mana1-3)Manb1-4GlcNAcb1-4GlcNAcb-Sp12 |
| 364 | Fuca1-4(Galb1-3)GlcNAcb1-2Mana1-6(Fuca1-4(Galb1-3)GlcNAcb1- |
| | 2Mana1-3)Manb1-4GlcNAcb1-4(Fuca1-6)GlcNAcb-Sp22 |
| 365 | Neu5Aca2-6GlcNAcb1-4GlcNAc-Sp21 |
| 366 | Neu5Aca2-6GlcNAcb1-4GlcNAcb1-4GlcNAc-Sp21 |
| 367 | Galb1-4(Fuca1-3)GlcNAcb1-6(Fuca1-2Galb1-4GlcNAcb1-3)Galb1-4Glc- |
| | Sp21 |
| 368 | Galb1-4GlcNAcb1-2Mana1-6(Galb1-4GlcNAcb1-4(Galb1-4GlcNAcb1- |
| | 2)Mana1-3)Manb1-4GlcNAcb1-4GlcNAc-Sp21 |
| 369 | GalNAca1-3(Fuca1-2)Galb1-4GlcNAcb1-2Mana1-6(GalNAca1-3(Fuca1- |
| | 2)Galb1-4GlcNAcb1-2Mana1-3)Manb1-4GlcNAcb1-4GlcNAcb-Sp20 |
| 370 | Gala1-3(Fuca1-2)Galb1-4GlcNAcb1-2Mana1-6(Gala1-3(Fuca1-2)Galb1- |
| | 4GlcNAcb1-2Mana1-3)Manb1-4GlcNAcb1-4GlcNAcb-Sp20 |
| 371 | Gala1-3Galb1-4(Fuca1-3)GlcNAcb1-2Mana1-6(Gala1-3Galb1-4(Fuca1- |
| | 3)GlcNAcb1-2Mana1-3)Manb1-4GlcNAcb1-4GlcNAcb-Sp20 |
| 372 | GalNAca1-3(Fuca1-2)Galb1-3GlcNAcb1-2Mana1-6(GalNAca1-3(Fuca1- |
| | 2)Galb1-3GlcNAcb1-2Mana1-3)Manb1-4GlcNAcb1-4GlcNAcb-Sp20 |
| 373 | Gala1-3(Fuca1-2)Galb1-3GlcNAcb1-2Mana1-6(Gala1-3(Fuca1-2)Galb1- |
| | 3GlcNAcb1-2Mana1-3)Manb1-4GlcNAcb1-4GlcNAcb-Sp20 |
| 374 | Fuca1-4(Fuca1-2Galb1-3)GlcNAcb1-2Mana1-3(Fuca1-4(Fuca1-2Galb1- |
| | 3)GlcNAcb1-2Mana1-3)Manb1-4GlcNAcb1-4GlcNAcb-Sp19 |
| 375 | Neu5Aca2-3Galb1-4GlcNAcb1-3GalNAc-Sp14 |
| 376 | Neu5Aca2-6Galb1-4GlcNAcb1-3GalNAc-Sp14 |
| 377 | Neu5Aca2-3Galb1-4(Fuca1-3)GlcNAcb1-3GalNAca-Sp14 |
| 378 | GalNAcb1-4GlcNAcb1-2Mana1-6(GalNAcb1-4GlcNAcb1-2Mana1-3)Manb1- |
| | 4GlcNAcb1-4GlcNAc-Sp12 |
| 379 | Galb1-3GalNAca1-3(Fuca1-2)Galb1-4Glc-Sp0 |
| 380 | Galb1-3GalNAca1-3(Fuca1-2)Galb1-4GlcNAc-Sp0 |
| 381 | Galb1-3GlcNAcb1-3Galb1-4GlcNAcb1-6(Galb1-3GlcNAcb1-3)Galb1-4Glcb- |
| | Sp0 |
| 382 | Galb1-4(Fuca1-3)GlcNAcb1-6(Galb1-3GlcNAcb1-3)Galb1-4Glc-Sp21 |
| 383 | Galb1-4GlcNAcb1-6(Fuca1-4(Fuca1-2Galb1-3)GlcNAcb1-3)Galb1-4Glc- |
| | Sp21 |
| 384 | Galb1-4(Fuca1-3)GlcNAcb1-6(Fuca1-4(Fuca1-2Galb1-3)GlcNAcb1-3)Galb1- |
| | 4Glc-Sp21 |
| 385 | Galb1-3GlcNAcb1-3Galb1-4(Fuca1-3)GlcNAcb1-6(Galb1-3GlcNAcb1- |
| | 3)Galb1-4Glc-Sp21 |
| 386 | Galb1-4GlcNAcb1-6(Galb1-4GlcNAcb1-2)Mana1-6(Galb1-4GlcNAcb1- |
| | 4(Galb1-4GlcNAcb1-2)Mana1-3)Manb1-4GlcNAcb1-4GlcNAcb-Sp21 |
| 387 | GlcNAcb1-2Mana1-6(GlcNAcb1-4(GlcNAcb1-2)Mana1-3)Manb1- |
| | 4GlcNAcb1-4GlcNAc-Sp21 |
| 388 | Fuca1-2Galb1-3GalNAca1-3(Fuca1-2)Galb1-4Glcb-Sp0 |
| 389 | Fuca1-2Galb1-3GalNAca1-3(Fuca1-2)Galb1-4GlcNAcb-Sp0 |
| 390 | Galb1-3GlcNAcb1-3GalNAca-Sp14 |
| 391 | GalNAcb1-4(Neu5Aca2-3)Galb1-4GlcNAcb1-3GalNAca-Sp14 |
| 392 | GalNAca1-3(Fuca1-2)Galb1-3GalNAca1-3(Fuca1-2)Galb1-4GlcNAcb-Sp0 |
| 393 | Gala1-3Galb1-3GlcNAcb1-2Mana1-6(Gala1-3Galb1-3GlcNAcb1-2Mana1- |
| | 3)Manb1-4GlcNAcb1-4GlcNAc-Sp19 |
| 394 | Gala1-3Galb1-3(Fuca1-4)GlcNAcb1-2Mana1-6(Gala1-3Galb1-3(Fuca1- |
| | 4)GlcNAcb1-2Mana1-3)Manb1-4GlcNAcb1-4GlcNAc-Sp19 |
| 395 | Neu5Aca2-3Galb1-3GlcNAcb1-2Mana1-6(Neu5Aca2-3Galb1-3GlcNAcb1- |
| | 2Mana1-3)Manb1-4GlcNAcb1-4GlcNAc-Sp19 |
| 396 | GlcNAcb1-2Mana1-6(Galb1-4GlcNAcb1-2Mana1-3)Manb1-4GlcNAcb1- |
| | 4GlcNAc-Sp12 |
| 397 | Galb1-4GlcNAcb1-2Mana1-6(GlcNAcb1-2Mana1-3)Manb1-4GlcNAcb1- |
| | 4GlcNAc-Sp12 |
| 398 | Neu5Aca2-3Galb1-3GlcNAcb1-3GalNAca-Sp14 |
| 399 | Fuca1-2Galb1-4GlcNAcb1-3GalNAca-Sp14 |
| 400 | Galb1-4(Fuca1-3)GlcNAcb1-3GalNAca-Sp14 |
| 401 | GalNAca1-3GalNAcb1-3Gala1-4Galb1-4GlcNAcb-Sp0 |
| 402 | Gala1-4Galb1-3GlcNAcb1-2Mana1-6(Gala1-4Galb1-3GlcNAcb1-2Mana1- |
| | 3)Manb1-4GlcNAcb1-4GlcNAcb-Sp19 |
| 403 | Gala1-4Galb1-4GlcNAcb1-2Mana1-6(Gala1-4Galb1-4GlcNAcb1-2Mana1- |
| | 3)Manb1-4GlcNAcb1-4GlcNAcb-Sp24 |
| 404 | Gala1-3Galb1-4GlcNAcb1-3GalNAca-Sp14 |
| 405 | Galb1-3GlcNAcb1-6Galb1-4GlcNAcb-Sp0 |
| 406 | Galb1-3GlcNAca1-6Galb1-4GlcNAcb-Sp0 |
| 407 | GalNAcb1-3Gala1-6Galb1-4Glcb-Sp8 |
| 408 | Gala1-3(Fuca1-2)Galb1-4(Fuca1-3)Glcb-Sp21 |
| 409 | Galb1-4GlcNAcb1-6(Neu5Aca2-6Galb1-3GlcNAcb1-3)Galb1-4Glc-Sp21 |
| 410 | Galb1-3GalNAcb1-4(Neu5Aca2-8Neu5Aca2-3)Galb1-4Glcb-Sp0 |
| 411 | Neu5Aca2-3Galb1-3GalNAcb1-4(Neu5Aca2-8Neu5Aca2-3)Galb1-4Glcb-Sp0 |
| 412 | Gala1-3(Fuca1-2)Galb1-4GlcNAcb1-3GalNAca-Sp14 |
| 413 | GalNAca1-3(Fuca1-2)Galb1-4GlcNAcb1-3GalNAca-Sp14 |
| 414 | GalNAca1-3GalNAcb1-3Gala1-4Galb1-4Glcb-Sp0 |
| 415 | Fuca1-2Galb1-4(Fuca1-3)GlcNAcb1-3GalNAca-Sp14 |
| 416 | Gala1-3(Fuca1-2)Galb1-4(Fuca1-3)GlcNAcb1-3GalNAc-Sp14 |
| 417 | GalNAca1-3(Fuca1-2)Galb1-4(Fuca1-3)GlcNAcb1-3GalNAc-Sp14 |
| 418 | Galb1-4(Fuca1-3)GlcNAcb1-2Mana1-6(Galb1-4(Fuca1-3)GlcNAcb1- |
| | 2Mana1-3)Manb1-4GlcNAcb1-4(Fuca1-6)GlcNAcb-Sp22 |
| 419 | Fuca1-2Galb1-4GlcNAcb1-2Mana1-6(Fuca1-2Galb1-4GlcNAcb1-2Mana1- |
| | 3)Manb1-4GlcNAcb1-4(Fuca1-6)GlcNAcb-Sp22 |
| 420 | GlcNAcb1-2(GlcNAcb1-6)Mana1-6(GlcNAcb1-2Mana1-3)Manb1- |
| | 4GlcNAcb1-4GlcNAcb-Sp19 |
| 421 | Fuca1-2Galb1-3GlcNAcb1-3GalNAc-Sp14 |
| 422 | Gala1-3(Fuca1-2)Galb1-3GlcNAcb1-3GalNAc-Sp14 |
| 423 | GalNAca1-3(Fuca1-2)Galb1-3GlcNAcb1-3GalNAc-Sp14 |
| 424 | Gala1-3Galb1-3GlcNAcb1-3GalNAc-Sp14 |
| 425 | Fuca1-2Galb1-3GlcNAcb1-2Mana1-6(Fuca1-2Galb1-3GlcNAcb1-2Mana1- |
| | 3)Manb1-4GlcNAcb1-4(Fuca1-6)GlcNAcb-Sp22 |
| 426 | Gala1-3(Fuca1-2)Galb1-4GlcNAcb1-2Mana1-6(Gala1-3(Fuca1-2)Galb1- |
| | 4GlcNAcb1-2Mana1-3)Manb1-4GlcNAcb1-4(Fuca1-6)GlcNAcb-Sp22 |
| 427 | Galb1-3GlcNAcb1-6(Galb1-3GlcNAcb1-2)Mana1-6(Galb1-3GlcNAcb1- |
| | 2Mana1-3)Manb1-4GlcNAcb1-4GlcNAcb-Sp19 |
| 429 | Galb1-4GlcNAcb1-6(Fuca1-2Galb1-3GlcNAcb1-3)Galb1-4Glc-Sp21 |
| 430 | Fuca1-3GlcNAcb1-6(Galb1-4GlcNAcb1-3)Galb1-4Glc-Sp21 |
| 430 | GlcNAcb1-2Mana1-6(GlcNAcb1-4)(GlcNAcb1-2Mana1-3)Manb1- |
| | 4GlcNAcb1-4GlcNAc-Sp21 |
| 431 | GlcNAcb1-2Mana1-6(GlcNAcb1-4)(GlcNAcb1-4(GlcNAcb1-2)Mana1- |
| | 3)Manb1-4GlcNAcb1-4GlcNAc-Sp21 |
| 432 | GlcNAcb1-6(GlcNAcb1-2)Mana1-6(GlcNAcb1-4)(GlcNAcb1-2Mana1- |
| | 3)Manb1-4GlcNAcb1-4GlcNAc-Sp21 |
| 433 | GlcNAcb1-6(GlcNAcb1-2)Mana1-6(GlcNAcb1-4)(GlcNAcb1-4(GlcNAcb1- |
| | 2)Mana1-3)Manb1-4GlcNAcb1-4GlcNAc-Sp21 |
| 434 | Galb1-4GlcNAcb1-2Mana1-6(GlcNAcb1-4)(Galb1-4GlcNAcb1-2Mana1- |
| | 3)Manb1-4GlcNAcb1-4GlcNAc-Sp21 |
| 435 | Galb1-4GlcNAcb1-2Mana1-6(GlcNAcb1-4)(Galb1-4GlcNAcb1-4(Galb1- |
| | 4GlcNAcb1-2)Mana1-3)Manb1-4GlcNAcb1-4GlcNAc-Sp21 |
| 436 | Galb1-4GlcNAcb1-6(Galb1-4GlcNAcb1-2)Mana1-6(GlcNAcb1-4)(Galb1- |
| | 4GlcNAcb1-2Mana1-3)Manb1-4GlcNAcb1-4GlcNAc-Sp21 |
| 437 | Galb1-4GlcNAcb1-6(Galb1-4GlcNAcb1-2)Mana1-6(GlcNAcb1-4)(Galb1- |
| | 4GlcNAcb1-4(Galb1-4GlcNAcb1-2)Mana1-3)Manb1-4GlcNAcb1-4GlcNAc- |
| | Sp21 |
| 438 | Galb1-4Galb-Sp10 |
| 439 | Galb1-6Galb-Sp10 |
| 440 | Neu5Aca2-3Galb1-4GlcNAcb1-3Galb-Sp8 |
| 441 | GalNAcb1-6GalNAcb-Sp8 |
| 442 | (6S)Galb1-3GlcNAcb-Sp0 |
| 443 | (6S)Galb1-3(6S)GlcNAc-Sp0 |
| 444 | Fuca1-2Galb1-4 GlcNAcb1-2Mana1-6(Fuca1-2Galb1-4GlcNAcb1-2(Fuca1- |
| | 2Galb1-4GlcNAcb1-4)Mana1-3)Manb1-4GlcNAcb1-4GlcNAcb-Sp12 |
| 445 | Fuca1-2Galb1-4(Fuca1-3)GlcNAcb1-2Mana1-6(Fuca1-2Galb1-4(Fuca1- |
| | 3)GlcNAcb1-4(Fuca1-2Galb1-4(Fuca1-3)GlcNAcb1-2)Mana1-3)Manb1- |
| | 4GlcNAcb1-4GlcNAcb-Sp12 |
| 446 | Galb1-4(Fuca1-3)GlcNAcb1-6GalNAc-Sp14 |
| 447 | Galb1-4GlcNAcb1-2Mana-Sp0 |
| 449 | Fuca1-2Galb1-4GlcNAcb1-6(Fuca1-2Galb1-4GlcNAcb1-3)GalNAc-Sp14 |
| 450 | Gala1-3Fuca1-2Galb1-4GlcNAcb1-6(Gala1-3Fuca1-2Galb1-4GlcNAcb1- |
| | 3)GalNAc-Sp14 |
| 451 | GalNAca1-3(Fuca1-2)Galb1-4GlcNAcb1-6(GalNAca1-3(Fuca1-2)Galb1- |
| | 4GlcNAcb1-3)GalNAc-Sp14 |
| 451 | Neu5Aca2-8Neu5Aca2-3Galb1-3GalNAcb1-4(Neu5Aca2-8Neu5Aca2- |
| | 3)Galb1-4Glcb-Sp0 |
| 452 | GalNAcb1-4Galb1-4Glcb-Sp0 |
| 453 | GalNAca1-3(Fuca1-2)Galb1-4GlcNAcb1-2Mana1-6(GalNAca1-3(Fuca1- |
| | 2)Galb1-4GlcNAcb1-2Mana1-3)Manb1-4GlcNAcb1-4(Fuca1-6)GlcNAcb- |
| | Sp22 |
| 454 | Gala1-3(Fuca1-2)Galb1-3GlcNAcb1-2Mana1-6(Gala1-3(Fuca1-2)Galb1- |
| | 3GlcNAcb1-2Mana1-3)Manb1-4GlcNAcb1-4(Fuca1-6)GlcNAcb-Sp22 |
| 455 | Neu5Aca2-6Galb1-4GlcNAcb1-6(Fuca1-2Galb1-3GlcNAcb1-3)Galb1-4Glc- |
| | Sp21 |
| 456 | GalNAca1-3(Fuca1-2)Galb1-3GlcNAcb1-2Mana1-6(GalNAca1-3(Fuca1- |
| | 2)Galb1-3GlcNAcb1-2Mana1-3)Manb1-4GlcNAcb1-4(Fuca1-6)GlcNAcb- |
| | Sp22 |
| 457 | Galb1-4GlcNAcb1-6(Galb1-4GlcNAcb1-2)Mana1-6(Galb1-4GlcNAcb1- |
| | 2Mana1-3)Manb1-4GlcNAcb1-4GlcNAcb-Sp19 |
| 458 | Neu5Aca2-3Galb1-4GlcNAcb1-2Mana1-6(GlcNAcb1-4)(Neu5Aca2-3Galb1- |
| | 4GlcNAcb1-2Mana1-3)Manb1-4GlcNAcb1-4GlcNAcb-Sp21 |
| 459 | Neu5Aca2-3Galb1-4GlcNAcb1-4Mana1-6(GlcNAcb1-4)(Neu5Aca2-3Galb1- |
| | 4GlcNAcb1-4(Neu5Aca2-3Galb1-4GlcNAcb1-2)Mana1-3)Manb1- |
| | 4GlcNAcb1-4GlcNAcb-Sp21 |
| 461 | Neu5Aca2-3Galb1-4GlcNAcb1-6(Neu5Aca2-3Galb1-4GlcNAcb1-2)Mana1- |
| | 6(GlcNAcb1-4)(Neu5Aca2-3Galb1-4GlcNAcb1-2Mana1-3)Manb1- |
| | 4GlcNAcb1-4GlcNAcb-Sp21 |
| 461 | Neu5Aca2-3Galb1-4GlcNAcb1-6(Neu5Aca2-3Galb1-4GlcNAcb1-2)Mana1- |
| | 6(GlcNAcb1-4)(Neu5Aca2-3Galb1-4GlcNAcb1-4(Neu5Aca2-3Galb1- |
| | 4GlcNAcb1-2)Mana1-3)Manb1-4GlcNAcb1-4GlcNAcb-Sp21 |
| 462 | Neu5Aca2-6Galb1-4GlcNAcb1-2Mana1-6(GlcNAcb1-4)(Neu5Aca2-6Galb1- |
| | 4GlcNAcb1-2Mana1-3)Manb1-4GlcNAcb1-4GlcNAcb-Sp21 |
| 463 | Neu5Aca2-6Galb1-4GlcNAcb1-4Mana1-6(GlcNAcb1-4)(Neu5Aca2-6Galb1- |
| | 4GlcNAcb1-4(Neu5Aca2-6Galb1-4GlcNAcb1-2)Mana1-3)Manb1- |
| | 4GlcNAcb1-4GlcNAcb-Sp21 |
| 464 | Neu5Aca2-6Galb1-4GlcNAcb1-6(Neu5Aca2-6Galb1-4GlcNAcb1-2)Mana1- |
| | 6(GlcNAcb1-4)(Neu5Aca2-6Galb1-4GlcNAcb1-2Mana1-3)Manb1- |
| | 4GlcNAcb1-4GlcNAcb-Sp21 |
| 465 | Neu5Aca2-6Galb1-4GlcNAcb1-6(Neu5Aca2-6Galb1-4GlcNAcb1-2)Mana1- |
| | 6(GlcNAcb1-4)(Neu5Aca2-6Galb1-4GlcNAcb1-4(Neu5Aca2-6Galb1- |
| | 4GlcNAcb1-2)Mana1-3)Manb1-4GlcNAcb1-4GlcNAcb-Sp21 |
| 466 | Gala1-3(Fuca1-2)Galb1-3GalNAca-Sp8 |
| 467 | Gala1-3(Fuca1-2)Galb1-3GalNAcb-Sp8 |
| 468 | Glca1-6Glca1-6Glca1-6Glcb-Sp10 |
| 469 | Glca1-4Glca1-4Glca1-4Glcb-Sp10 |
| 470 | Neu5Aca2-3Galb1-4GlcNAcb1-6(Neu5Aca2-3Galb1-4GlcNAcb1- |
| | 3)GalNAca-Sp14 |
| 471 | Fuca1-2Galb1-4(Fuca1-3)GlcNAcb1-2Mana1-6(Fuca1-2Galb1-4(Fuca1- |
| | 3)GlcNAcb1-2Mana1-3)Manb1-4GlcNAcb1-4(Fuca1-6)GlcNAcb-Sp24 |
| 472 | Fuca1-2Galb1-3(Fuca1-4)GlcNAcb1-2Mana1-6(Fuca1-2Galb1-3(Fuca1- |
| | 4)GlcNAcb1-2Mana1-3)Manb1-4GlcNAcb1-4(Fuca1-6)GlcNAcb1-4(Fuca1- |
| | 6)GlcNAcb-Sp19 |
| 473 | Neu5Aca2-3Galb1-3GlcNAcb1-6(Neu5Aca2-3Galb1-4GlcNAcb1-2)Mana1- |
| | 6(Neu5Aca2-3Galb1-3GlcNAcb1-2Mana1-3)Manb1-4GlcNAcb1-4GlcNAcb- |
| | Sp19 |
| 474 | GlcNAcb1-6(GlcNAcb1-2)Mana1-6(GlcNAcb1-2Mana1-3)Manb1- |
| | 4GlcNAcb1-4(Fuca1-6)GlcNAcb-Sp24 |
| 475 | Galb1-3GlcNAcb1-2Mana1-6(GlcNAcb1-4)(Galb1-3GlcNAcb1-2Mana1- |
| | 3)Manb1-4GlcNAcb1-4GlcNAcb-Sp21 |
| 476 | Neu5Aca2-6Galb1-4GlcNAcb1-6(Galb1-3GlcNAcb1-3)Galb1-4Glcb-Sp21 |
| 477 | Neu5Aca2-3Galb1-4GlcNAcb1-2Mana-Sp0 |
| 478 | Neu5Aca2-3Galb1-4GlcNAcb1-6GalNAca-Sp14 |
| 479 | Neu5Aca2-6Galb1-4GlcNAcb1-6GalNAca-Sp14 |
| 480 | Neu5Aca2-6Galb1-4 GlcNAcb1-6(Neu5Aca2-6Galb1-4GlcNAcb1- |
| | 3)GalNAca-Sp14 |
| 481 | Neu5Aca2-6Galb1-4GlcNAcb1-2Mana1-6(Neu5Aca2-6Galb1-4GlcNAcb1- |
| | 2Mana1-3)Manb1-4GlcNAcb1-4(Fuca1-6)GlcNAcb-Sp24 |
| 482 | Neu5Aca2-3Galb1-4GlcNAcb1-2Mana1-6(Neu5Aca2-3Galb1-4GlcNAcb1- |
| | 2Mana1-3)Manb1-4GlcNAcb1-4(Fuca1-6)GlcNAcb-Sp24 |
| 483 | Mana1-6(Mana1-3)Manb1-4GlcNAcb1-4(Fuca1-6)GlcNAcb-Sp19 |
| 484 | Galb1-4GlcNAcb1-6(Galb1-4GlcNAcb1-2)Mana1-6(Galb1-4GlcNAcb1- |
| | 2Mana1-3)Manb1-4GlcNAcb1-4(Fuca1-6)GlcNAcb-Sp24 |
| 485 | Neu5Aca2-3Galb1-3GlcNAcb1-2Mana1-6(GlcNAcb1-4)(Neu5Aca2-3Galb1- |
| | 3GlcNAcb1-2Mana1-3)Manb1-4GlcNAcb1-4GlcNAc-Sp21 |
| 486 | Neu5Aca2-6Galb1-4GlcNAcb1-6(Fuca1-2Galb1-4(Fuca1-3)GlcNAcb1- |
| | 3)Galb1-4Glc-Sp21 |
| 487 | Galb1-3GlcNAcb1-6GalNAca-Sp14 |
| 488 | Gala1-3Galb1-3GlcNAcb1-6GalNAca-Sp14 |
| 489 | Galb1-3(Fuca1-4)GlcNAcb1-6GalNAca-Sp14 |
| 490 | Neu5Aca2-3Galb1-3GlcNAcb1-6GalNAca-Sp14 |
| 491 | (3S)Galb1-3(Fuca1-4)GlcNAcb-Sp0 |
| 493 | Galb1-4(Fuca1-3)GlcNAcb1-6(Neu5Aca2-6(Neu5Aca2-3Galb1-3)GlcNAcb1- |
| | 3)Galb1-4Glc-Sp21 |
| 493 | Fuca1-2Galb1-4GlcNAcb1-6GalNAca-Sp14 |
| 494 | Gala1-3Galb1-4GlcNAcb1-6GalNAca-Sp14 |
| 495 | Galb1-4(Fuca1-3)GlcNAcb1-2Mana-Sp0 |
| 496 | Fuca1-2(6S)Galb1-3GlcNAcb-Sp0 |
| 497 | Gala1-3(Fuca1-2)Galb1-4GlcNAcb1-6GalNAca-Sp14 |
| 498 | Fuca1-2Galb1-4GlcNAcb1-2Mana-Sp0 |
| 499 | Fuca1-2Galb1-3(6S)GlcNAcb-Sp0 |
| 500 | Fuca1-2(6S)Galb1-3(6S)GlcNAcb-Sp0 |
| 501 | Neu5Aca2-6GalNAcb1-4(6S)GlcNAcb-Sp8 |
| 502 | GalNAcb1-4(Fuca1-3)(6S)GlcNAcb-Sp8 |
| 503 | (3S)GalNAcb1-4(Fuca1-3)GlcNAcb-Sp8 |
| 504 | Fuca1-2Galb1-3GlcNAcb1-6(Fuca1-2Galb1-3GlcNAcb1-3)GalNAca-Sp14 |
| 505 | GalNAca1-3(Fuca1-2)Galb1-3GlcNAcb1-6GalNAca-Sp14 |
| 506 | GlcNAcb1-6(GlcNAcb1-2)Mana1-6(GlcNAcb1-4)(GlcNAcb1-4(GlcNAcb1- |
| | 2)Mana1-3)Manb1-4GlcNAcb1-4(Fuca1-6)GlcNAc-Sp21 |
| 508 | Galb1-4GlcNAcb1-6(Galb1-4GlcNAcb1-2)Mana1-6(GlcNAcb1-4)Galb1- |
| | 4GlcNAcb1-4(Gal b1-4GlcNAcb1-2)Mana1-3)Manb1-4GlcNAcb1-4(Fuca1- |
| | 6)GlcNAc-Sp21 |
| 508 | Galb1-3GlcNAca1-3Galb1-4GlcNAcb-Sp8 |
| 509 | Galb1-3(6S)GlcNAcb-Sp8 |
| 510 | (6S)(4S)GalNAcb1-4GlcNAc-Sp8 |
| 511 | (6S)GalNAcb1-4GlcNAc-Sp8 |
| 512 | (3S)GalNAcb1-4(3S)GlcNAc-Sp8 |
| 513 | GalNAcb1-4(6S)GlcNAc-Sp8 |
| 514 | (3S)GalNAcb1-4GlcNAc-Sp8 |
| 515 | (4S)GalNAcb-Sp10 |
| 516 | Galb1-4(6P)GlcNAcb-Sp0 |
| 517 | (6P)Galb1-4GlcNAcb-SP0 |
| 518 | GalNAca1-3(Fuca1-2)Galb1-4GlcNAcb1-6GalNAc-Sp14 |
| 519 | Neu5Aca2-6Galb1-4GlcNAcb1-2Man-Sp0 |
| 520 | Gala1-3Galb1-4GlcNAcb1-2Mana-Sp0 |
| 521 | Gala1-3(Fuca1-2)Galb1-4GlcNAcb1-2Mana-Sp0 |
| 522 | GalNAca1-3(Fuca1-2)Galb1-4GlcNAcb1-2Mana-Sp0 |
| 523 | Galb1-3GlcNAcb1-2Mana-Sp0 |
| 524 | Gala1-3(Fuca1-2)Galb1-3GlcNAcb1-6GalNAc-Sp14 |
| 525 | Neu5Aca2-3Galb1-3GlcNAcb1-2Mana-Sp0 |
| 526 | Gala1-3Galb1-3GlcNAcb1-2Mana-Sp0 |
| 527 | GalNAcb1-4GlcNAcb1-2Mana-Sp0 |
| 528 | Neu5Aca2-3Galb1-3GlcNAcb1-4Galb1-4Glcb-Sp0 |
| 529 | GlcNAcb1-2Mana1-6(GlcNAcb1-4)(GlcNAcb1-2Mana1-3)Manb1- |
| | 4GlcNAcb1-4(Fuca1-6)GlcNAc-Sp21 |
| 530 | Galb1-4GlcNAcb1-2Mana1-6(GlcNAcb1-4)(Galb1-4GlcNAcb1-2Mana1- |
| | 3)Manb1-4GlcNAcb1-4(Fuca1-6)GlcNAc-Sp21 |
| 531 | Galb1-4GlcNAcb1-2Mana1-6(Galb1-4GlcNAcb1-4)(Galb1-4GlcNAcb1- |
| | 2Mana1-3)Manb1-4GlcNAcb1-4(Fuca1-6)GlcNAc-Sp21 |
| 532 | Fuca1-4(Galb1-3)GlcNAcb1-2Mana-Sp0 |
| 533 | Neu5Aca2-3Galb1-4(Fuca1-3)GlcNAcb1-2Mana-Sp0 |
| 534 | GlcNAcb1-3Galb1-4GlcNAcb1-6(GlcNAcb1-3)Galb1-4GlcNAc-Sp0 |
| 535 | GalNAca1-3(Fuca1-2)Galb1-3GalNAcb1-3Gala1-4Galb1-4Glc-Sp21 |
| 536 | Gala1-3(Fuca1-2)Galb1-3GalNAcb1-3Gala1-4Galb1-4Glc-Sp21 |
| 537 | Galb1-3GalNAcb1-3Gal-Sp21 |
| 538 | GlcNAcb1-3Galb1-4GlcNAcb1-2Mana1-6(GlcNAcb1-3Galb1-4GlcNAcb1- |
| | 2Mana1-3)Manb1-4GlcNAcb1-4GlcNAcb-Sp12 |
| 539 | GlcNAcb1-3Galb1-4GlcNAcb1-2Mana1-6(GlcNAcb1-3Galb1-4GlcNAcb1- |
| | 2Mana1-3)Manb1-4GlcNAcb1-4GlcNAcb-Sp25 |
| 540 | Galb1-4GlcNAcb1-3Galb1-4GlcNAcb1-2Mana1-6(Galb1-4GlcNAcb1- |
| | 3Galb1-4GlcNAcb1-2Mana1-3)Manb1-4GlcNAcb1-4GlcNAcb-Sp12 |
| 541 | Galb1-4GlcNAcb1-3Galb1-4GlcNAcb1-2Mana1-6(Galb1-4GlcNAcb1- |
| | 3Galb1-4GlcNAcb1-2Mana1-3)Manb1-4GlcNAcb1-4GlcNAcb-Sp24 |
| 542 | Neu5Gca2-3Galb1-4GlcNAcb1-3Galb1-4GlcNAcb1-2Mana1-6(Neu5Gca2- |
| | 3Galb1-4GlcNAcb1-3Galb1-4GlcNAcb1-2Mana1-3)Manb1-4GlcNAcb1- |
| | 4GlcNAcb-Sp24 |
| 543 | Fuca1-2Galb1-4GlcNAcb1-3Galb1-4GlcNAcb1-2Mana1-6(Fuca1-2Galb1- |
| | 4GlcNAcb1-3Galb1-4GlcNAcb1-2Mana1-3)Manb1-4GlcNAcb1-4GlcNAcb- |
| | Sp24 |
| 544 | GlcNAcb1-3Galb1-4GlcNAcb1-3Galb1-4GlcNAcb1-2Mana1-6(GlcNAcb1- |
| | 3Galb1-4GlcNAcb1-3Galb1-4GlcNAcb1-2Mana1-3)Manb1-4GlcNAcb1- |
| | 4GlcNAcb-Sp12 |
| 545 | GlcNAcb1-3Galb1-4GlcNAcb1-3Galb1-4GlcNAcb1-2Mana1-6(GlcNAcb1- |
| | 3Galb1-4GlcNAcb1-3Galb1-4GlcNAcb1-2Mana1-3)Manb1-4GlcNAcb1- |
| | 4GlcNAcb-Sp25 |
| 546 | Galb1-4GlcNAcb1-3Galb1-4GlcNAcb1-3Galb1-4GlcNAcb1-2Mana1- |
| | 6(Galb1-4GlcNAcb1-3Galb1-4GlcNAcb1-3Galb1-4GlcNAcb1-2Mana1- |
| | 3)Manb1-4GlcNAcb1-4GlcNAcb-Sp12 |
| 547 | Galb1-4GlcNAcb1-3Galb1-4GlcNAcb1-3Galb1-4GlcNAcb1-2Mana1- |
| | 6(Galb1-4GlcNAcb1-3Galb1-4GlcNAcb1-3Galb1-4GlcNAcb1-2Mana1- |
| | 3)Manb1-4GlcNAcb1-4GlcNAcb-Sp24 |
| 548 | GlcNAcb1-3Galb1-4GlcNAcb1-3Galb1-4GlcNAcb1-3Galb1-4GlcNAcb1- |
| | 2Mana1-6(GlcNAcb1-3Galb1-4GlcNAcb1-3Galb1-4GlcNAcb1-3Galb1- |
| | 4GlcNAcb1-2Mana1-3)Manb1-4GlcNAcb1-4GlcNAcb-Sp25 |
| 549 | Galb1-4GlcNAcb1-3Galb1-4GlcNAcb1-3Galb1-4GlcNAcb1-3Galb1- |
| | 4GlcNAcb1-2Mana1-6(Galb1-4GlcNAcb1-3Galb1-4GlcNAcb1-3Galb1- |
| | 4GlcNAcb1-3Galb1-4GlcNAcb1-2Mana1-3)Manb1-4GlcNAcb1-4GlcNAcb- |
| | Sp25 |
| 550 | Galb1-3GlcNAcb1-3Galb1-4GlcNAcb1-2Mana1-6(Galb1-3GlcNAcb1- |
| | 3Galb1-4GlcNAcb1-2Mana1-3)Manb1-4GlcNAcb1-4GlcNAc-Sp25 |
| 551 | Neu5Gca2-8Neu5Gca2-3Galb1-4GlcNAc-Sp0 |
| 552 | Neu5Aca2-8Neu5Gca2-3Galb1-4GlcNAc-Sp0 |
| 553 | Neu5Gca2-8Neu5Aca2-3Galb1-4GlcNAc-Sp0 |
| 554 | Neu5Gca2-8Neu5Gca2-3Galb1-4GlcNAcb1-3Galb1-4GlcNAc-Sp0 |
| 555 | Neu5Gca2-8Neu5Gca2-6Galb1-4GlcNAc-Sp0 |
| 556 | Neu5Aca2-8Neu5Aca2-3Galb1-4GlcNAc-Sp0 |
| 557 | GlcNAcb1-3Galb1-4GlcNAcb1-6(GlcNAcb1-3Galb1-4GlcNAcb1-2)Mana1- |
| | 6(GlcNAcb1-3Galb1-4GlcNAcb1-2Man a1-3)Manb1-4GlcNAcb1-4GlcNAc- |
| | Sp24 |
| 558 | Galb1-4GlcNAcb1-3Galb1-4GlcNAcb1-6(Galb1-4GlcNAcb1-3Galb1- |
| | 4GlcNAcb1-2)Mana1-6(Galb1-4GlcNAcb1-3Galb1-4GlcNAcb1-2Mana1- |
| | 3)Mana1-4GlcNAcb1-4GlcNAc-Sp24 |
| 559 | Gala1-3Galb1-4GlcNAcb1-2Mana1-6(Gala1-3Galb1-4GlcNAcb1-2Mana1- |
| | 3)Manb1-4GlcNAcb1-4GlcNAc-Sp24 |
| 560 | GlcNAcb1-3Galb1-4GlcNAcb1-6(GlcNAcb1-3Galb1-3)GalNAca-Sp14 |
| 561 | GalNAcb1-3GlcNAcb-Sp0 |
| 562 | GalNAcb1-4GlcNAcb1-3GalNAcb1-4GlcNAcb-Sp0 |
| 563 | GlcNAcb1-3Galb1-4GlcNAcb1-3Galb1-4GlcNAcb1-3Galb1-4GlcNAcb1- |
| | 3Galb1-4GlcNAcb1-2Mana1-6(GlcNAcb1-3Galb1-4GlcNAcb1-3Galb1- |
| | 4GlcNAcb1-3Galb1-4GlcNAcb1-3Galb1-4GlcNAcb1-2Mana1-3)Manb1- |
| | 4GlcNAcb1-4GlcNAcb-Sp25 |
| 564 | Galb1-4GlcNAcb1-3Galb1-4GlcNAcb1-3Galb1-4GlcNAcb1-3Galb1- |
| | 4GlcNAcb1-3Galb1-4GlcNAcb1-2Mana1-6(Galb1-4GlcNAcb1-3Galb1- |
| | 4GlcNAcb1-3Galb1-4GlcNAcb1-3Galb1-4GlcNAcb1-3Galb1-4GlcNAcb1- |
| | 2Mana1-3)Manb1-4GlcNAcb1-4GlcNAcb-Sp25 |
| 565 | GlcNAb1-3Galb1-3GalNAc-Sp14 |
| 566 | Galb1-3GlcNAcb1-6(Galb1-3)GalNAc-Sp14 |
| 567 | Galb1-4GlcNAcb1-3Galb1-4GlcNAcb1-3Galb1-4GlcNAcb1-3Galb1- |
| | 4GlcNAcb1-3Galb1-4GlcNAcb1-3Galb1-4GlcNAcb1-2Mana1-6(Galb1- |
| | 4GlcNAcb1-3Galb1-4GlcNAcb1-3Galb1-4GlcNAcb1-3Galb1-4GlcNAcb1- |
| | 3Galb1-4GlcNAcb1-3Galb1-4GlcNAcb1-2Mana1-3)Manb1-4GlcNAcb1- |
| | 4GlcNAcb-Sp25 |
| 568 | (3S)GlcAb1-3Galb1-4GlcNAcb1-3Galb1-4Glc-Sp0 |
| 569 | (3S)GlcAb1-3Galb1-4GlcNAcb1-2Mana-Sp0 |
| 570 | Galb1-3GlcNAcb1-3Galb1-4GlcNAcb1-3Galb1-4GlcNAcb1-6(Galb1- |
| | 3GlcNAcb1-3Galb1-4GlcNAcb1-3Galb1-4GlcNAb1-2)Mana1-6(Galb1- |
| | 3GlcNAcb1-3Galb1-4GlcNAcb1-3Galb1-4GlcNAcb1-2Mana1-3)Manb1- |
| | 4GlcNAcb1-4(Fuca1-6)GlcNAcb-Sp24 |
| 571 | Galb1-3GlcNAcb1-3Galb1-4GlcNAcb1-6(Galb1-3GlcNAcb1-3Galb1- |
| | 4GlcNAb1-2)Mana1-6(Galb1-3GlcNAcb1-3Galb1-4GlcNAcb1-2Mana1- |
| | 3)Manb1-4GlcNAcb1-4(Fuca1-6)GlcNAcb-Sp24 |
| 572 | Neu5Aca2-8Neu5Aca2-3Galb1-3GalNAcb1-4(Neu5Aca2-3)Galb1-4Glc-Sp21 |
| 573 | GlcNAcb1-3Galb1-4GlcNAcb1-2Mana1-6(GlcNAcb1-3Galb1-4GlcNAcb1- |
| | 2Mana1-3)Manb1-4GlcNAcb1-4(Fuca1-6)GlcNAcb-Sp24 |
| 574 | Galb1-4GlcNAcb1-3Galb1-4GlcNAcb1-2Mana1-6(Galb1-4GlcNAcb1- |
| | 3Galb1-4GlcNAcb1-2Mana1-3)Manb1-4GlcNAcb1-4(Fuca1-6)GlcNAcb- |
| | Sp24 |
| 575 | GlcNAcb1-3Galb1-4GlcNAcb1-3Galb1-4GlcNAcb1-2Mana1-6(GlcNAcb1- |
| | 3Galb1-4GlcNAcb1-3Galb1-4GlcNAcb1-2Mana1-3)Manb1-4GlcNAcb1- |
| | 4(Fuca1-6)GlcNAcb-Sp24 |
| 576 | Galb1-4GlcNAcb1-3Galb1-4GlcNAcb1-3Galb1-4GlcNAcb1-2Mana1- |
| | 6(Galb1-4GlcNAcb1-3Galb1-4GlcNAcb1-3Galb1-4GlcNAcb1-2Mana1- |
| | 3)Manb1-4GlcNAcb1-4(Fuca1-6)GlcNAcb-Sp24 |
| 577 | GlcNAcb1-3Galb1-4GlcNAcb1-3Galb1-4GlcNAcb1-3Galb1-4GlcNAcb1- |
| | 2Mana1-6(GlcNAcb1-3Galb1-4GlcNAcb1-3Galb1-4GlcNAcb1-3Galb1- |
| | 4GlcNAcb1-2Mana1-3)Manb1-4GlcNAcb1-4(Fuca1-6)GlcNAcb-Sp24 |
| 578 | Galb1-4GlcNAcb1-3Galb1-4GlcNAcb1-3Galb1-4GlcNAcb1-3Galb1- |
| | 4GlcNAcb1-2Mana1-6(Galb1-4GlcNAcb1-3Galb1-4GlcNAcb1-3Galb1- |
| | 4GlcNAcb1-3Galb1-4GlcNAcb1-2Mana1-3)Manb1-4GlcNAcb1-4(Fuca1- |
| | 6)GlcNAcb-Sp24 |
| 579 | GlcNAcb1-3Galb1-4GlcNAcb1-3Galb1-4GlcNAcb1-3Galb1-4GlcNAcb1- |
| | 3Galb1-4GlcNAcb1-2Mana1-6(GlcNAcb1-3Galb1-4GlcNAcb1-3Galb1- |
| | 4GlcNAcb1-3Galb1-4GlcNAcb1-3Galb1-4GlcNAcb1-2Mana1-3)Manb1- |
| | 4GlcNAcb1-4(Fuca1-6)GlcNAcb-Sp19 |
| 580 | Galb1-4GlcNAcb1-3Galb1-4GlcNAcb1-3Galb1-4GlcNAcb1-3Galb1- |
| | 4GlcNAcb1-3Galb1-4GlcNAcb1-2Mana1-6(Galb1-4GlcNAcb1-3Galb1- |
| | 4GlcNAcb1-3Galb1-4GlcNAcb1-3Galb1-4GlcNAcb1-3Galb1-4GlcNAcb1- |
| | 2Mana1-3)Manb1-4GlcNAcb1-4(Fuca1-6)GlcNAcb-Sp19 |
| 581 | Galb1-4GlcNAcb1-3Galb1-4GlcNAcb1-6(Galb1-4GlcNAcb1-3Galb1- |
| | 4GlcNAb1-2)Mana1-6(Galb1-4GlcNAcb1-3Galb1-4GlcNAcb1-2Mana1- |
| | 3)Manb1-4GlcNAcb1-4(Fuca1-6)GlcNAcb-Sp24 |
| 582 | GlcNAcb1-3Galb1-4GlcNAcb1-3Galb1-4GlcNAcb1-6(GlcNAcb1-3Galb1- |
| | 4GlcNAcb1-3Galb1-4GlcNAb1-2)Mana1-6(GlcNAcb1-3Galb1-4GlcNAcb1- |
| | 3Galb1-4GlcNAcb1-2Mana1-3)Manb1-4GlcNAcb1-4(Fuca1-6)GlcNAcb- |
| | Sp24 |
| 583 | Galb1-4GlcNAcb1-3Galb1-4GlcNAcb1-3Galb1-4GlcNAcb1-6(Galb1- |
| | 4GlcNAcb1-3Galb1-4GlcNAcb1-3Galb1-4GlcNAb1-2)Mana1-6(Galb1- |
| | 4GlcNAcb1-3Galb1-4GlcNAcb1-3Galb1-4GlcNAcb1-2Mana1-3)Manb1- |
| | 4GlcNAcb1-4(Fuca1-6)GlcNAcb-Sp24 |
| 584 | GlcNAcb1-3Galb1-4GlcNAcb1-3Galb1-4GlcNAcb1-3Galb1-4GlcNAcb1- |
| | 6(GlcNAcb1-3Galb1-4GlcNAcb1-3Galb1-4GlcNAcb1-3Galb1-4GlcNAb1- |
| | 2)Mana1-6(GlcNAcb1-3Galb1-4GlcNAcb1-3Galb1-4GlcNAcb1-3Galb1- |
| | 4GlcNAcb1-2Mana1-3)Manb1-4GlcNAcb1-4(Fuca1-6)GlcNAcb-Sp24 |
| 585 | Galb1-4GlcNAcb1-3Galb1-4GlcNAcb1-3Galb1-4GlcNAcb1-3Galb1- |
| | 4GlcNAcb1-6(Galb1-4GlcNAcb1-3Galb1-4GlcNAcb1-3Galb1-4GlcNAcb1- |
| | 3Galb1-4GlcNAb1-2)Mana1-6(Galb1-4GlcNAcb1-3Galb1-4GlcNAcb1- |
| | 3Galb1-4GlcNAcb1-3Galb1-4GlcNAcb1-2Mana1-3)Manb1-4GlcNAcb1- |
| | 4(Fuca1-6)GlcNAcb-Sp24 |
| 586 | GlcNAcb1-3Galb1-4GlcNAcb1-3Galb1-4GlcNAcb1-3Galb1-4GlcNAcb1- |
| | 3Galb1-4GlcNAcb1-6(GlcNAcb1-3Galb1-4GlcNAcb1-3Galb1-4GlcNAcb1- |
| | 3Galb1-4GlcNAcb1-3Galb1-4GlcNAb1-2)Mana1-6(GlcNAcb1-3Galb1- |
| | 4GlcNAcb1-3Galb1-4GlcNAcb1-3Galb1-4GlcNAcb1-3Galb1-4GlcNAcb1- |
| | 2Mana1-3)Manb1-4GlcNAcb1-4(Fuca1-6)GlcNAcb-Sp24 |
| 587 | Galb1-4GlcNAcb1-3Galb1-4GlcNAcb1-3Galb1-4GlcNAcb1-3Galb1- |
| | 4GlcNAcb1-3Galb1-4GlcNAcb1-6(Galb1-4GlcNAcb1-3Galb1-4GlcNAcb1- |
| | 3Galb1-4GlcNAcb1-3Galb1-4GlcNAcb1-3Galb1-4GlcNAb1-2)Mana1- |
| | 6(Galb1-4GlcNAcb1-3Galb1-4GlcNAcb1-3Galb1-4GlcNAcb1-3Galb1- |
| | 4GlcNAcb1-3Galb1-4GlcNAcb1-2Mana1-3)Manb1-4GlcNAcb1-4(Fuca1- |
| | 6)GlcNAcb-Sp24 |
| 588 | Galb1-4GlcNAcb1-3Galb1-4GlcNAcb1-3GalNAca-Sp14 |
| 589 | Galb1-4GlcNAcb1-3Galb1-4GlcNAcb1-6(Galb1-3)GalNAca-Sp14 |
| 590 | Galb1-4GlcNAcb1-3Galb1-4GlcNAcb1-6(Galb1-4GlcNAcb1-3Galb1- |
| | 4GlcNAcb1-3)GalNAca-Sp14 |
| 591 | Neu5Aca2-3Galb1-4GlcNAcb1-3Galb1-4GlcNAcb1-3GalNAca-Sp14 |
| 592 | GlcNAcb1-3Galb1-4GlcNAcb1-3GalNAca-Sp14 |
| 593 | GlcNAcb1-3Galb1-4GlcNAcb1-6(Galb1-3)GalNAca-Sp14 |
| 594 | GlcNAcb1-3Galb1-4GlcNAcb1-6(GlcNAcb1-3Galb1-4GlcNAcb1- |
| | 3)GalNAca-Sp14 |
| 595 | Neu5Aca2-3Galb1-4GlcNAcb1-3Galb1-4GlcNAcb1-6(Neu5Aca2-3Galb1- |
| | 4GlcNAcb1-3Galb1-4GlcNAcb1-3)GalNAca-Sp14 |
| 596 | Neu5Aca2-6Galb1-4GlcNAcb1-3Galb1-4GlcNAcb1-3GalNAca-Sp14 |
| 597 | GlcNAcb1-3Galb1-4GlcNAcb1-3Galb1-4GlcNAcb1-3GalNAca-Sp14 |
| 598 | Galb1-4GlcNAcb1-3Galb1-3GalNAca-Sp14 |
| 599 | Neu5Aca2-3Galb1-4GlcNAcb1-3Galb1-4GlcNAcb1-6(Galb1-3)GalNAca- |
| | Sp14 |
| 600 | Neu5Aca2-6Galb1-4GlcNAcb1-3Galb1-4GlcNAcb1-6(Galb1-3)GalNAca- |
| | Sp14 |
| 601 | Neu5Aca2-6Galb1-4GlcNAcb1-6(Galb1-3)GalNAca-Sp14 |
| 602 | Neu5Aca2-3Galb1-4GlcNAcb1-3Galb1-4GlcNAcb1-2Mana1-6(Neu5Aca2- |
| | 3Galb1-4GlcNAcb1-3Galb1-4GlcNAcb1-2Mana1-3)Manb1-4GlcNAcb1- |
| | 4GlcNAcb-Sp12 |
| 603 | GlcNAcb1-6(Neu5Aca2-3Galb1-3)GalNAca-Sp14 |
| 604 | Neu5Aca2-6Galb1-4GlcNAcb1-3Galb1-4GlcNAcb1-6(Neu5Aca2-6Galb1- |
| | 4GlcNAcb1-3Galb1-4GlcNAcb1-3)GalNAca-Sp14 |
| 605 | Neu5Aca2-6Galb1-4GlcNAcb1-3Galb1-4GlcNAcb1-3Galb1-4GlcNAcb1- |
| | 2Mana1-6(Neu5Aca2-6Galb1-4GlcNAcb1-3Galb1-4GlcNAcb1-3Galb1- |
| | 4GlcNAcb1-2Mana1-3)Manb1-4GlcNAcb1-4GlcNAcb-Sp12 |
| 606 | Neu5Aca2-3Galb1-4GlcNAcb1-3Galb1-4GlcNAcb1-3Galb1-4GlcNAcb1- |
| | 2Mana1-6(Neu5Aca2-3Galb1-4GlcNAcb1-3Galb1-4GlcNAcb1-3Galb1- |
| | 4GlcNAcb1-2Mana1-3)Manb1-4GlcNAcb1-4GlcNAcb-Sp12 |
| 607 | Neu5Aca2-6Galb1-4GlcNAcb1-3Galb1-4GlcNAcb1-2Mana1-6(Neu5Aca2- |
| | 6Galb1-4GlcNAcb1-3Galb1-4GlcNAcb1-2Mana1-3)Manb1-4GlcNAcb1- |
| | 4GlcNAcb-Sp12 |
| 608 | GlcNAcb1-3Fuca-Sp21 |
| 609 | Galb1-3GalNAcb1-4(Neu5Aca2-8Neu5Aca2-8Neu5Aca2-3)Galb1-4Glcb- |
| | Sp21 |
| 596 | Neu5Aca2-6Galb1-4GlcNAcb1-3Galb1-4GlcNAcb1-3GalNAca-Sp14 |

TABLE 2
Structures of the slide attachment linkers.

| Linker | Structure |
|---|---|
| Sp0 | CH₂CH₂NH₂ |
| Sp8 | CH₂CH₂CH₂NH₂ |
| Sp9 | CH₂CH₂CH₂CH₂CH₂N_{H2} |
| Sp10 | NHCOCH₂NH |
| Sp11 | OCH₂C₆H₄-p-NHCOCH₂NH |
| Sp12 | Asparagine |
| Sp13 | Glycine |
| Sp14 | Threonine |
| Sp15 | Serine |
| Sp16 | PNP (OC₆H₄NH₂) |
| Sp17 | OCH₂C₆H₄NH₂ |
| Sp18 | O(CH₂)₃NHCO(CH₂)₅NH₂ |
| Sp19 | EN or NK |
| Sp20 | GENR |
| Sp21 | —N(CH3)—O—(CH2)2—NH2 |
| Sp22 | NST |
| Sp23 | (OCH2CH2)6NH2 |
| Sp24 | KVANKT |
| Sp25 | VANK |
| MDPLys | Mur-L-Ala-D-iGlnb-(CH2)4NH2 |

### Example 4 Statistical Analysis

Statistical analysis was performed using GraphPad Prism (GraphPad Software Inc., version 6.0). Graphics were generated using both Microsoft Excel 2007 and GraphPad. Data were normalized by dividing each value by the median value of the sample to account for varying concentrations of IgA. Statistical comparisons were performed using a two-tailed unpaired t-test in GraphPad. Area under curve (AUC) values were also determined using GraphPad.

### Example 5 LC-MS/MS Analysis of GLF and Identification of IgA as being Predominant in GLF

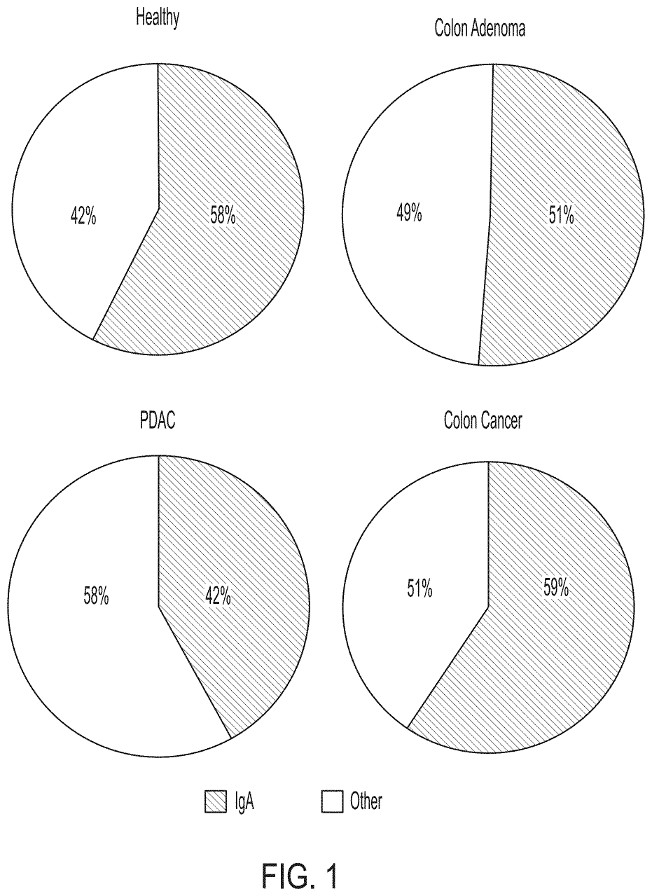

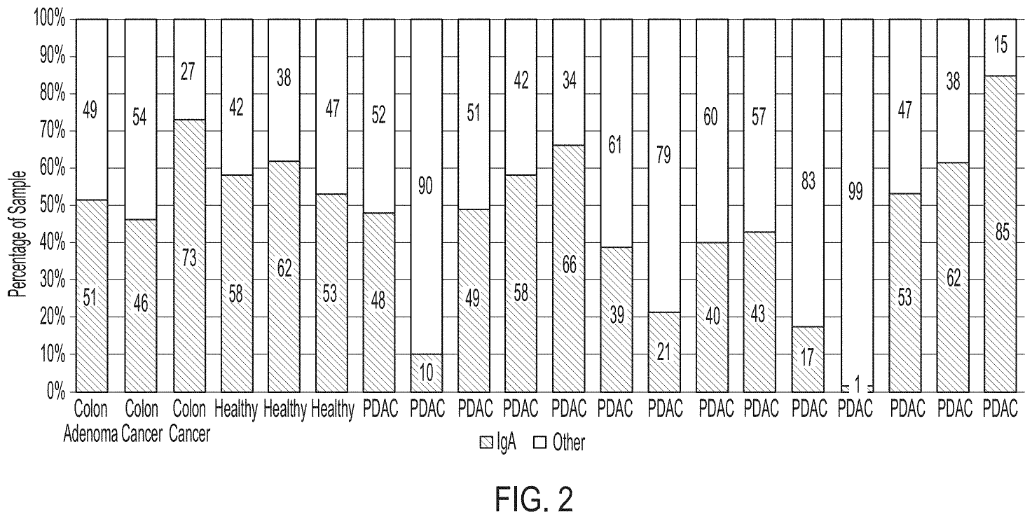

Preliminary work performed on GLF revealed that IgA comprises a large fraction of the protein content as determined by spectral counting from liquid chromatography-tandem mass spectrometry (LC-MS/MS). These data are shown in FIG. 1. The present study focuses on the binding profile of PDAC patient IgA. To show that the PDAC glycan immunosignature is unique, various other conditions needed to be investigated. The conditions investigated were PDAC (n=14), healthy controls (n=3), colon cancer (n=2), and colon adenoma (n=1). Potentially confounding diseases were accounted for by focusing on another localized cancer and its potentially precancerous lesions. These non-PDAC samples were used to show that the selective targeting appears to be PDAC specific and related to specific anti-glycan antibodies associated with the disease. The IgA composition of each of the 20 samples used, shown in FIG. 2, in this study does not indicate a change in overall antibody levels between conditions.

Under normal circumstances the level of IgA is still very high compared to other proteins based on the mass spectrometry data shown in FIG. 1. Although it is possible that an increase in the antibody could occur during disease state, it is likely not enough of a change to alter the overall composition in GLF. The rise will likely be only in a subset of clones that have begun producing affinity matured IgA. This proposed surge in specific IgA is capitalized on here in this work. Data in FIGS. 2A-2C, which are discussed in greater detail in Example 6, show that the highly-abundant IgA in GLF is also functional.

As IgA levels are high in GLF, this immune response-based approach is more practical than others focusing on immunoglobulins that are found in low abundance not only in GLF, but also in the overall immune system, such as IgE and IgD. IgG is the most abundant antibody in circulation but is very low to non-existent at mucosal surfaces. IgA is actively transported across the epithelial barrier of the intestine. MS data has confirmed very low amounts of IgG in GLF. It is seen most abundantly in GLF when blood proteins are present, which would then indicate bleeding. The IgG present may bind to the glycans on the array, but if it were to compete against the IgA-glycan, no signals would be seen. Even if IgG may be present in GLF, the IgG-glycan binding should not form a noise related to the IgA signal as anti-IgA antibodies were used to detect IgA binding. The data presented herein are not from purified IgA, but from diluted or crude GLF. IgG and IgA can be differentiated by MS and on the glycan array by anti-IgG or anti-IgA secondary antibodies. Only anti-IgA secondary antibodies were used on the array.

This research allowed the inventors to see if GLF was a valid medium that contained specific IgAs resulting from cancer or a potential cause of cancer. The glycan arrays used in this study are a limited resource as the usage was allocated by the Consortium of Functional Glycomics (CFG) steering committee in maximum batches of ten samples per request.

While pooling of patients' GLF is also possible, this might complicate the trend of these targeted IgAs across the set of samples, even if it would allow the inventors to examine a larger number. Thus, the glycan targets were better observed from an individual patient and not masked by the results of others.

### Example 6 Differential IgA-Glycan Binding Profiles Across Multiple Conditions

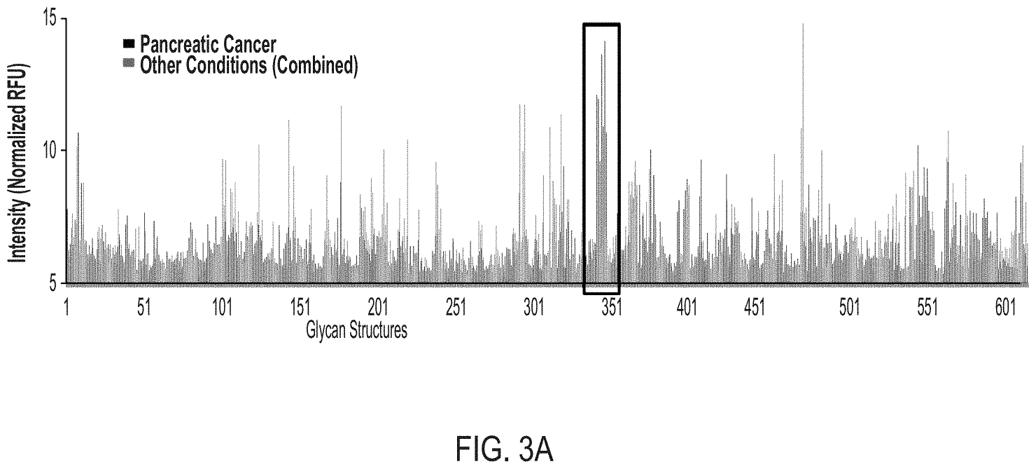

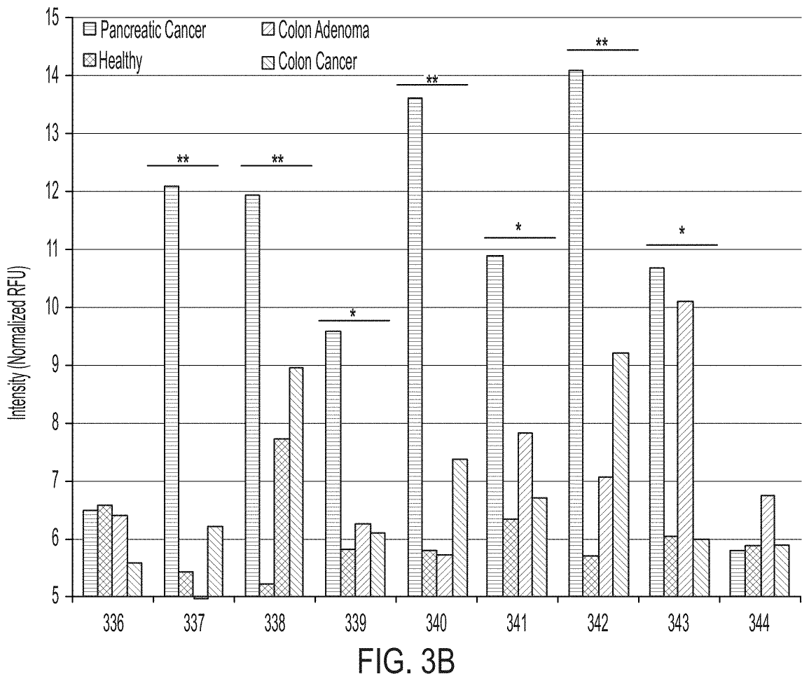

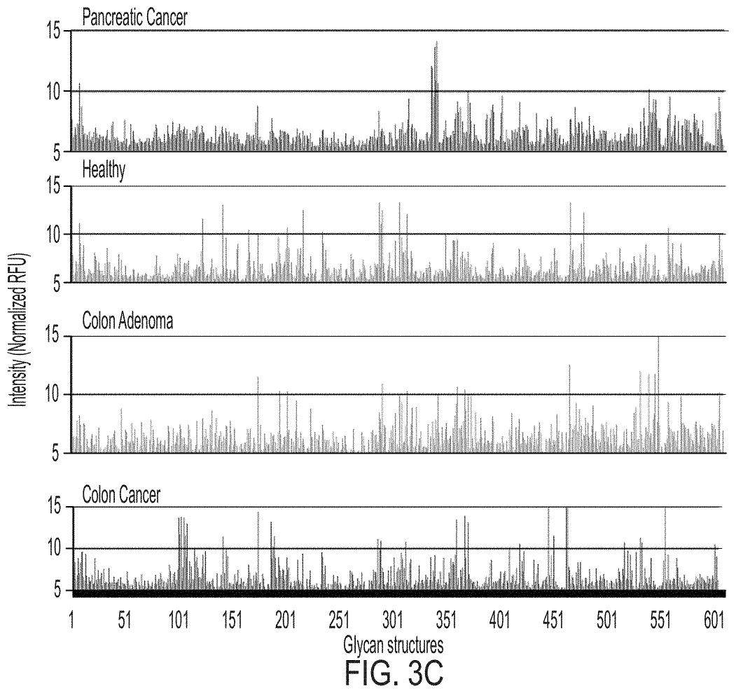

Each of these patient samples were screened against the CFG (www.functionalglycomics.org) glycan array v5.2 composed of 609 unique glycan structures. Overall glycan binding is presented in FIG. 3A with an enlarged region of interest in FIG. 3B. All samples have been normalized to the same median relative fluorescence units (RFU) value and displayed on the y-axis. A higher RFU value corresponds to increased IgA binding to a specific structure. Without wishing to be bound by theory, the inventors attribute the measured RFU values to correspond to the amount of IgA immunoglobulin molecules associated with the glycan as well as the binding affinity of IgA to one or more specific glycans. Accordingly, RFU values can increase with an increase of IgA that binds a glycan structure and can also increase with an increased binding affinity of IgA towards the glycan structure. The x-axes of the plots are the 609 individual carbohydrate structures where each number represents a unique glycan structure. The total binding in each condition has been averaged together across all samples of the same condition in FIG. 3C. This helps to show an overall IgA-glycan immunosignature for each condition.

As is visually evident in FIG. 3C, the overall signatures of glycans bound by patient IgA differ by subject diagnosis. To further investigate differences and commonalities between and within patient groups, heat maps were generated. The heat map allows for simple visual interpretation of these data where differences in binding are represented across a color spectrum. Several distinct groupings appear after visual inspection of FIG. 4A. PDAC patient antibodies seem to react with a different set of structures than healthy, colon cancer, and colon adenoma patients. There are a set of structures where PDAC patients show increased reactivity and another set where PDAC patients showed reduced binding as compared to other conditions.

These observations are interesting as they provide PDAC distinction from other conditions. In addition, although the number of patients with potentially confounding conditions is low, it appears that there are distinct signatures associated specifically with PDAC. This is an important control in this study as the ability to distinguish PDAC from other GI tract cancers and controls shows that the response is likely disease-specific.

The unique signatures also appear to be disease-specific. Specifically, the distinct binding profiles, as depicted in FIGS. 2A-2C, add to the evidence that affinity matured antibodies, like IgA, are capable of binding carbohydrates. There is a broad spectrum of binding overall which would be expected as the many targets bound by intestinal IgA includes various glycan structures. Additionally, the array platform may contain mimitopes, structures that mimic other epitopes, leading to reactivity to glycans that may not have been the original target of the antibodies.

### Example 7 Structural Relationships of IgA-Reactive and Non-IgA-Reactive Glycans and Glycan Motifs for PDAC

Two-tailed t-tests were used to compare PDAC versus non-PDAC patient samples across individual glycan structures, as outlined in Example 4. Monosaccharide composition and linkages as well as overall glycan structure were thoroughly investigated for the top 8 most significantly different structures with p≤1.27e-6. The set of 8 structures by slide number are: Glycan ID NOs: 337, 338, 340, 194, 291, 314, 202, 342 (listed in order of increasing p value). The group contains 4 structures that showed increased binding in PDAC patients (Glycan ID NOs: 337, 338, 340, 342) and 4 structures with decreased binding (Glycan ID NOs: 194, 291, 314, 202).

As the glycans were inspected, structural similarities where PDAC-patient IgA binding was increased became evident. The structures 337, 340, 342, and 338 are displayed in FIG. 4B. The similar binding among PDAC patients is a strong indication that there are immunological reactions associated with PDAC specifically in mucosa-associated lymphoid tissue (MALT) IgA. The terminating motif of GlcNAcα1-4Galβ1-4GlcNAc is present on 337, 340, 342, and 338 further adding to the consistency and specificity of the results. Whether or not this motif is present within the gut is uncertain, but the structure along with possible origins are in Example 8. The carbohydrate may share antigenic similarities with another entity that elicits an immune response during PDAC development.

### Example 8 IgA-Glycan-Binding Pattern Analysis Across Multiple Conditions

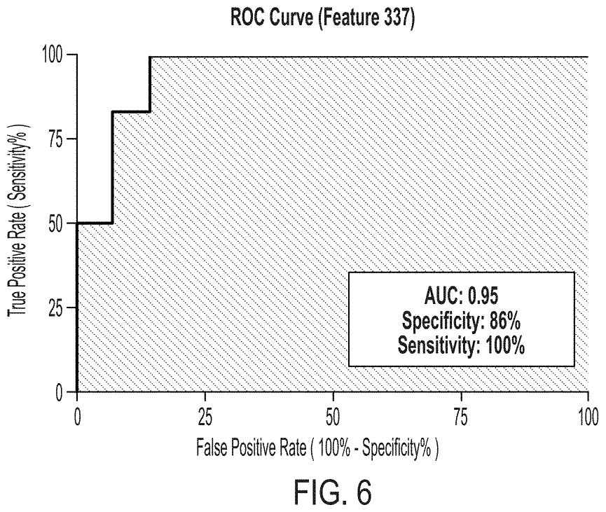

Structural comparisons of glycans that demonstrated the highest and lowest binding intensity in each sample group representing different conditions were performed. The top 10 highest and lowest intensity binders were examined for structural similarities and arranged in FIGS. 5A-5B to most effectively show structurally-related glycan motifs. Sensitivity and specificity of the IgA-binding of each glycan is displayed in the format of a receiver operating characteristic curve (ROC) curve. For example, the ROC curve for the PDAC GLF IgA binding of Glycan ID NO: 337 (see Table 1 for structure) of FIG. 6 shows an area-under-curve (AUC) value of 0.95 for combined specificity and selectivity, or individually, 86% specificity and 100% selectivity.

Distinct patterns that were observed in the glycans are distinguished by the boxes with dashed lines. Each sample group revealed marked differences in the glycans that were the highest intensity binders with the exception of the highest intensity binders in the healthy samples which did not display any common patterns.

The lowest intensity binders showed distinct patterns within each sample group. The trend was also present across sample groups, most notably the presence of terminal sialic acid (Neu5Ac). A lack of reactivity to Neu5Ac has been documented using similar glycan arrays testing IgG-glycan interactions as reported in Schneider et al. (2015, Sci. Transl. Med., 7, 269ra1). Terminal Neu5Ac residues are common in human glycoproteins. As Schneider et al. states, “Indeed, Neu5Ac is a self-antigen that is ubiquitously expressed as the terminal moiety on many glycoproteins and glycolipids in human tissues”. Immunological tolerance is likely at play in the development of class-switched antibodies such as IgA in the present studies and the previously reported IgG research. It would be expected that without this tolerance autoimmune conditions would arise. The similar results showing a lack of reactivity to terminal sialic acids published by Schneider et al. is important as the healthy patients in this study showed the same characteristics corroborating the results shown here for non-PDAC samples.

A structure that appears to be differentially reactive among PDAC patients versus other conditions is GlcNAcα1-4Galβ1-4GlcNAc, which has already been identified above in Example 7. According to the Carbohydrate Structure Database (csdb.glycoscience.ru), the motif is a component of the lipopolysaccharide from Proteus mirabilis, a bacterial species commonly found in the gut.

While a number of embodiments of this invention have been described in the foregoing examples, it is apparent that these basic examples may be altered to provide other embodiments that utilize the compounds and methods of this disclosure. Therefore, it will be appreciated that the scope of this disclosure is to be defined by the appended claims rather than by the specific embodiments that have been represented by way of example.

The contents of all references (including literature references with their respective supplementary materials, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A method for diagnosing pancreatic cancer or predisposition thereto in a subject, comprising:
obtaining a sample comprising gastrointestinal lavage fluid (GLF) from the subject, wherein the GLF comprises immunoglobulin A (IgA);
contacting the GLF with a plurality of glycans; and
detecting binding between the IgA and the plurality of glycans, thereby detecting a glycan-binding pattern; wherein the glycan-binding pattern is associated with pancreatic cancer or predisposition thereto.

2. The method according to claim 1, wherein the glycan-binding pattern comprises
(a) at least one glycan associated with pancreatic cancer or predisposition thereto,
(b) at least two glycans associated with pancreatic cancer or predisposition thereto;
(c) a glycan motif found in at least one glycan associated with pancreatic cancer or a predisposition thereto; optionally, wherein the glycan motif is GlcNAcα1-4Galβ1-4GlcNAc;
(d) at least one glycan associated with pancreatic cancer or a predisposition thereto at a statistical significance testing p-value of ≤1.00e-6; and/or
(e) at least one glycan associated with pancreatic cancer or a predisposition thereto with an area under curve (AUC) value of a receiver operating characteristic (ROC) curve of at least 0.95.

3-4. (canceled)

5. The method according to claim 2, wherein
(a) the at least one glycan is pre-determined to be bound at the highest intensity by the IgA in GLF from pancreatic cancer patients;
(b) the at least two glycans are pre-determined to be bound at the highest and the second highest intensities by the IgA in GLF from pancreatic cancer patients;
(c) the at least one glycan or the at least two glycans have a binding intensity of higher than 10 RFU;
(d) the at least one glycan or the at least two glycans are at at least the 90th percentile of glycan IgA binding intensities;
(e) the at least one glycan is pre-determined to be bound at the lowest intensity by the IgA in GLF from pancreatic cancer patients;
(f) the at least two glycans are pre-determined to be bound at the lowest and the second lowest intensities by the IgA in GLF from pancreatic cancer patients;
(g) the at least one glycan or the at least two glycans have a binding intensity of no higher than 7 RFU;
(h) the at least one glycan or the at least two glycans are at or below the 10th percentile of glycan IgA binding intensities;
(i) the at least one glycan or the at least two glycans are selected from the group consisting of: GlcNAcα1-4Galβ1-4GlcNAcβ1-3Galβ1-4GlcNAcβ1-3Galβ1-4GlcNAc;
GlcNAcα1-4Galβ1-4GlcNAc;
GlcNAcα1-4Galβ1-4GlcNAcβ1-3Galβ1-4Glc;
GlcNAcα1-4Galβ1-4GlcNAcβ1-3Galβ1-4(Fucα1-3)GlcNAcβ1-3Galβ1-4(Fucα1-3)GlcNAc;
GlcNAcα1-4Galβ1-4GlcNAcβ1-3Galβ1-4GlcNAc; and
GlcNAcβ1-3Galβ1-4GlcNAcβ1-3Galβ1-4GlcNAcβ1-2Manα1-6(GlcNAcβ1-3Galβ1-4GlcNAcβ1-3Galβ1-4GlcNAcβ1-2Manα1-3)Manβ1-4GlcNAcβ1-4GlcNAc; and/or
(j) the at least one glycan or the at least two glycans are selected from the group consisting of: GlcNAcβ1-6Galβ1-4GlcNAc;
GlcA;
Galβ1-4(Fucα1-3)(6S)Glc; and
Manα1-6(Manα1-3)Manα1-6(Manα1-3)Man.

6-17. (canceled)

18. The method according to claim 1, wherein the plurality of glycans
(a) are exogenous glycans;
(b) does not comprise an endogenous carbohydrate cancer antigen; and/or
(c) does not comprise the carbohydrate cancer antigen CA19-9.

19-20. (canceled)

21. The method according to claim 1, wherein the pancreatic cancer is selected from the group consisting of pancreatic ductal adenocarcinoma (PDAC), adenosquamous carcinoma, squamous cell carcinoma, giant cell carcinoma, acinar cell carcinoma, small cell carcinoma, islet cell tumors, pancreas endocrine tumors (PETs), pancreatic neuroendocrine tumors (PNETs), insulinomas, glucagonomas, somatostatinomas, gastrinomas, VlPomas (arising from vasoactive intestinal peptide-making cells) and non-secreting islet tumors of the pancreas.

22. The method according to claim 1, wherein the pancreatic cancer is pancreatic ductal adenocarcinoma (PDAC).

23. The method according to claim 1, further comprising
(a) administering a lavage composition to the subject to thereby induce purgation of the subject's gastrointestinal tract and/or to cleanse the subject's gastrointestinal tract wherein the lavage composition
(i) is administered orally;
(ii) is a solution or a solid composition that is reconstituted in a solvent prior to being administered to the subject;
(iii) comprises an active ingredient selected from group consisting of polyethylene glycol (PEG), magnesium sulfate, sodium sulfate, potassium sulfate, magnesium citrate, sodium citrate, potassium citrate, ascorbic acid, citric acid, sodium picosulfate, sodium chloride, potassium chloride, bisacodyl, magnesium oxide, magnesium phosphate, potassium phosphate, sodium phosphate, monobasic sodium phosphate, dibasic sodium phosphate, potassium hydrogen tartrate, magnesium carbonate, magnesium hydroxide, sodium hydroxide, potassium hydroxide, sodium carbonate, sodium bicarbonate, ammonium carbonate, an anhydrous form thereof, a hydrate thereof, or a combination thereof; and/or
(iv) comprises: a PEG having a molecular weight of between about 3000 and about 4000, sodium sulfate, sodium bicarbonate, sodium chloride, and potassium chloride; or
a PEG having a molecular weight of between about 3000 and about 4000, sodium bicarbonate, sodium chloride, and potassium chloride; or
sodium sulfate, magnesium sulfate, and potassium sulfate; or
monobasic sodium phosphate and dibasic sodium phosphate, or a hydrate thereof; and/or
(b) separating the GLF from the sample; wherein the GLF does not comprise solid or semisolid fecal matter, fecal proteins and fecal immunoglobulins.

24-30. (canceled)

31. The method according to claim 1, wherein the binding between the IgA and the plurality of glycans is detected and measured using an immunoassay, a chemiluminescent assay, a fluorescent assay, a colorimetric assay, or a mass spectrometry assay.

32. A method for detecting a glycan-binding pattern associated with pancreatic cancer or predisposition thereto in a subject, comprising:
obtaining a sample comprising gastrointestinal lavage fluid (GLF) from the subject, wherein the GLF comprises immunoglobulin A (IgA);
contacting the GLF with a plurality of glycans; and
detecting binding between the IgA and the plurality of glycans, thereby detecting the glycan-binding pattern.

33-62. (canceled)

63. A method for evaluating or monitoring the progression of pancreatic cancer or the efficacy of a pancreatic cancer treatment in a subject via a glycan-binding pattern associated with pancreatic cancer or predisposition thereto, comprising:
obtaining a sample comprising gastrointestinal lavage fluid (GLF) from the subject, wherein the GLF comprises immunoglobulin A (IgA);
contacting the GLF with a plurality of glycans; and
detecting binding between the IgA and the plurality of glycans, thereby detecting the glycan-binding pattern.

64-94. (canceled)

95. A method for diagnosing a gastrointestinal (GI) tract cancer or predisposition thereto in a subject, comprising:
obtaining a sample comprising gastrointestinal lavage fluid (GLF) from the subject, wherein the GLF comprises immunoglobulin A (IgA);
contacting the GLF with a plurality of glycans; and
detecting binding between the IgA and the plurality of glycans, thereby detecting a glycan-binding pattern; wherein the glycan-binding pattern is associated with the GI tract cancer or predisposition thereto.

96. The method according to claim 95, wherein the glycan-binding pattern comprises
(a) at least one glycan associated with the GI cancer or predisposition thereto;
(b) at least two glycans associated with the GI cancer or predisposition thereto;
(c) a glycan motif found in at least one glycan associated with the GI cancer or a predisposition thereto; optionally, wherein the glycan motif is
(i) GlcNAcα1-4Galβ1-4GlcNAc;
(ii) one or more of the following: Galα1-3Galβ1-4GlcNAc; Galα1-3Galβ1-4(Fuc); and Galα1-3Galβ1-4(Fucα1-3)GlcNAc; and/or
(iii) one or more of the following: Galβ1-4GlcNAcβ1-3Galβ1-4GlcNAc; and Galβ1-4GlcNAc;
(d) at least one glycan associated with the GI cancer or a predisposition thereto at a statistical significance testing p-value of ≤1.00e-6; and/or
(e) at least one glycan associated with the GI cancer or a predisposition thereto with an area under curve (AUC) value of a receiver operating characteristic (ROC) curve of at least 0.95.

97-98. (canceled)

99. The method according to claim 96, wherein
(a) the at least one glycan is pre-determined to be bound at the highest intensity by the IgA in GLF from the GI cancer patients;
(b) the at least two glycans are pre-determined to be bound at the highest and the second highest intensities by the IgA in GLF from the GI cancer patients;
(c) the at least one glycan or the at least two glycans have a binding intensity of higher than 10 RFU;
(d) the at least one glycan or the at least two glycans are at at least the 90th percentile of glycan IgA binding intensities;
(e) the at least one glycan is pre-determined to be bound at the lowest intensity by the IgA in GLF from the GI cancer patients;
(f) the at least two glycans are pre-determined to be bound at the lowest and the second lowest intensities by the IgA in GLF from the GI cancer patients;
(g) the at least one glycan or the at least two glycans have a binding intensity of no higher than 7 RFU;
(h) the at least one glycan or the at least two glycans are at or below the 10th percentile of glycan IgA binding intensities;
(i) the at least one glycan or the at least two glycans are selected from the group consisting of: GlcNAcα1-4Galβ1-4GlcNAcβ1-3Galβ1-4GlcNAcβ1-3Galβ1-4GlcNAc;
GlcNAcα1-4Galβ1-4GlcNAc;
GlcNAcα1-4Galβ1-4GlcNAcβ1-3Galβ1-4Glc;
GlcNAcα1-4Galβ1-4GlcNAcβ1-3Galβ1-4(Fucα1-3)GlcNAcβ1-3Galβ1-4(Fucα1-3)GlcNAc;
GlcNAcα1-4Galβ1-4GlcNAcβ1-3Galβ1-4GlcNAc; and
GlcNAcβ1-3Galβ1-4GlcNAcβ1-3Galβ1-4GlcNAcβ1-2Manα1-6(GlcNAcβ1-3Galβ1-4GlcNAcβ1-3Galβ1-4GlcNAcβ1-2Manα1-3)Manβ1-4GlcNAcβ1-4GlcNAc;
(i) the at least one glycan or the at least two glycans are selected from the group consisting of: GlcNAcβ1-6Galβ1-4GlcNAc;
GlcA;
Galβ1-4(Fucα1-3)(6S)Glc; and
Manα1-6(Manα1-3)Manα1-6(Manα1-3)Man,
(k) the at least one glycan or the at least two glycans are selected from the group consisting of: Galα1-3Galβ1-4(Fucα1-3)GlcNAc;
Galα1-3Galβ1-4GlcNAcβ1-2Manα1-6(Galα1-3Galβ1-4GlcNAcβ1-2Manα1-3)Manβ1-4GlcNAcβ1-4GlcNAc;
Galα1-3Galβ1-4(Fucα1-3)GlcNAcβ1-2(Galα1-3Galβ1-4(Fucα1-3)GlcNAcβ1-2GalNAc;
Galα1-3Galβ1-4GlcNAcβ1-2Manα1-6(Galα1-3Galβ1-4GlcNAcβ1-2Manα1-3)Manβ1-4GlcNAcβ1-4GlcNAc;
Galα1-3Galβ1-4(Fucα1-3)GalNAc;
Galα1-3Galβ1-4(Fucα1-3)GlcNAcβ1-2Manα1-6(Galα1-3Galβ1-4(Fucα1-3)GlcNAc; and
Galα1-3Galβ1-4(Fucα1-3)GlcNAc; and/or
(l) the at least one glycan is selected from the group consisting of:
Fucα1-2Galβ1-4(Fucα1-3)GlcNAcβ1-2Manα1-6(Fucα1-2Galβ1-4(Fucα1-3)GlcNAcβ1-2Manα1-3)Manβ1-4GlcNAcβ1-4GlcNAc;
Galβ1-4GlcNAcβ1-6(Galβ1-4GlcNAcβ1-2)Manα1-6(Galβ1-4GlcNAcβ1-2Manα1-3)Manβ1-4GlcNAcβ1-4GlcNAc;
Galβ1-4GlcNAcβ1-3Galβ1-4GlcNAcβ1-3Galβ1-4GlcNAcβ1-2Manα1-6(Galβ1-4GlcNAcβ1-3Galβ1-4GlcNAcβ1-3Galβ1-4GlcNAcβ1-2Manα1-3)Manβ1-4GlcNAcβ1-4GlcNAc;
Galβ1-4GlcNAcβ1-3Galβ1-4GlcNAcβ1-3Galβ1-4GlcNAcβ1-3Galβ1-4GlcNAcβ1-2Manα1-6(Galβ1-4GlcNAcβ1-3Galβ1-4GlcNAcβ1-3Galβ1-4GlcNAcβ1-3Galβ1-4GlcNAcβ1-2Manα1-3)Manβ1-4GlcNAcβ1-4GlcNAc; and
Galβ1-4(Fucα1-3)GlcNAc.

100-106. (canceled)

107. The method according to claim 95, wherein the GI tract cancer is
(a) pancreatic cancer; wherein the pancreatic cancer is selected from the group consisting of pancreatic ductal adenocarcinoma (PDAC), adenosquamous carcinoma, squamous cell carcinoma, giant cell carcinoma, acinar cell carcinoma, small cell carcinoma, islet cell tumors, pancreas endocrine tumors (PETS), pancreatic neuroendocrine tumors (PNETs), insulinomas, glucagonomas, somatostatinomas, gastrinomas, VlPomas (arising from vasoactive intestinal peptide-making cells) and non-secreting islet tumors of the pancreas;
(b) colon cancer; and/or
(c) colon adenoma.

108. (canceled)

109. The method according to claim 107, wherein the pancreatic cancer is pancreatic ductal adenocarcinoma (PDAC).

110-120. (canceled)

121. The method according to claim 95, wherein the plurality of glycans
(a) are exogenous glycans;
(b) does not comprise an endogenous carbohydrate cancer antigen; and/or
(c) does not comprise the carbohydrate cancer antigen CA19-9.

122-123. (canceled)

124. The method according to claim 95, further comprising
(a) administering a lavage composition to the subject to thereby induce purgation of the subject's gastrointestinal tract and/or to cleanse the subject's gastrointestinal tract wherein the lavage composition
(i) is administered orally;
(ii) is a solution or a solid composition that is reconstituted in a solvent prior to being administered to the subject;
(iii) comprises an active ingredient selected from group consisting of polyethylene glycol (PEG), magnesium sulfate, sodium sulfate, potassium sulfate, magnesium citrate, sodium citrate, potassium citrate, ascorbic acid, citric acid, sodium picosulfate, sodium chloride, potassium chloride, bisacodyl, magnesium oxide, magnesium phosphate, potassium phosphate, sodium phosphate, monobasic sodium phosphate, dibasic sodium phosphate, potassium hydrogen tartrate, magnesium carbonate, magnesium hydroxide, sodium hydroxide, potassium hydroxide, sodium carbonate, sodium bicarbonate, ammonium carbonate, an anhydrous form thereof, a hydrate thereof, or a combination thereof; and/or
(iv) comprises: a PEG having a molecular weight of between about 3000 and about 4000, sodium sulfate, sodium bicarbonate, sodium chloride, and potassium chloride; or
a PEG having a molecular weight of between about 3000 and about 4000, sodium bicarbonate, sodium chloride, and potassium chloride; or
sodium sulfate, magnesium sulfate, and potassium sulfate; or
monobasic sodium phosphate and dibasic sodium phosphate, or a hydrate thereof; and/or
(b) separating the GLF from the sample; wherein the GLF does not comprise solid or semisolid fecal matter, fecal proteins and fecal immunoglobulins.

125-131. (canceled)

132. The method according to claim 95, wherein the binding between the IgA and the plurality of glycans is detected and measured using an immunoassay, a chemiluminescent assay, a fluorescent assay, a colorimetric assay, or a mass spectrometry assay.

133. A method for detecting a glycan-binding pattern associated with a gastrointestinal (GI) tract cancer or predisposition thereto in a subject, or evaluating or monitoring the progression of GI tract cancer or the efficacy of GI cancer treatment comprising:
obtaining a sample comprising gastrointestinal lavage fluid (GLF) from the subject, wherein the GLF comprises immunoglobulin A (IgA);
contacting the GLF with a plurality of glycans; and
detecting binding between the IgA and the plurality of glycans, thereby detecting the glycan-binding pattern.

134-195. (canceled)
